(19) United States
(12) Patent Application Publication
(10) Pub. No.: US 2024/0391956 A1
(43) Pub. Date: Nov. 28, 2024

(54) STAPLED PEPTIDES AND METHODS THEREOF

(71) Applicant: FOG PHARMACEUTICALS, INC., Cambridge, MA (US)

(72) Inventors: Olena Tokareva, Andover, MA (US); Kunhua Li, Newton, MA (US); Tara Lynne Travaline, Arlington, MA (US); John Hanney McGee, Somerville, MA (US)

(21) Appl. No.: 18/427,677

(22) Filed: Jan. 30, 2024

Related U.S. Application Data
(60) Provisional application No. 63/482,159, filed on Jan. 30, 2023.

(51) Int. Cl. C07K 7/08; A61K 38/00

(52) U.S. Cl. CPC C07K 7/08; A61K 38/00

(57) ABSTRACT

Among other things, the present disclosure provides various useful agents. In some embodiments, provided agents can bind to MDM2. In some embodiments, provided agents can bind to CHIP. In some embodiments, the present disclosure provides technologies for modulating MDM2 functions. In some embodiments, the present disclosure provides technologies for modulating CHIP functions. In some embodiments, the present disclosure provides technologies for preventing or treating conditions, disorders or diseases.

### CROSS-REFERENCE TO RELATED APPLICATIONS

This application claims priority to U.S. Provisional Application No. 63/482,159, filed Jan. 30, 2023, the entirety of which is incorporated herein by reference.

### SEQUENCE LISTING

The instant application contains a Sequence Listing which has been submitted electronically in XML format and is hereby incorporated by reference in its entirety. Said XML copy, created on Aug. 9, 2024, is named SL.xml and is 43,083 bytes in size.

### BACKGROUND

Stapled peptides are useful for various applications. For example, as biologically active agents, they can be utilized to modulate various biological functions.

### SUMMARY

Among other things, the present disclosure provides powerful technologies (e.g., agents (e.g., those that are or comprise peptides, in many embodiments, stapled peptides), compositions, methods, etc.) for modulating various biological functions, e.g., of E3 ubiquitin-protein ligase Mdm2 (MDM2; Uniprot ID: Q00987) or E3 ubiquitin-protein ligase CHIP (CHIP; Uniprot ID: Q9UNE7), or fragment thereof. In some embodiments, the present disclosure provides agents, e.g., stapled peptides, that can bind MDM2 or a fragment thereof. In some embodiments, the present disclosure provides agents, e.g., stapled peptides, that can bind CHIP or a fragment thereof. In some embodiments, provided agents, e.g., stapled peptides, comprises one or more (e.g., 1, 2, 3, 4, 5, 6, 7, 8, 9, 10, 11, 12, 13, 14, 15, 16, 17, 18, 19, 20 or more; or 10%, 15%, 20%, 25%, 30%, 35%, 40%, 45%, 50%, 55%, 60%, 65%, 70%, 75%, 80%, 85%, 90%, or 100% of all amino acid residues) D-amino acid residues. In some embodiments, provided agents, e.g., stapled peptides, comprises one or more consecutive (e.g., 1, 2, 3, 4, 5, 6, 7, 8, 9, 10, 11, 12, 13, 14, 15, 16, 17, 18, 19, 20 or more; or 10%, 15%, 20%, 25%, 30%, 35%, 40%, 45%, 50%, 55%, 60%, 65%, 70%, 75%, 80%, 85%, 90%, or 100% of all amino acid residues) D-amino acid residues.

In some embodiments, the provided technology provides an agent, e.g., a stapled peptide, that comprises one or more staples within 10-20, 10-15, 10, 11, 12, 13, 14, 15, 16, 17, 18, 19, or 20 consecutive amino acids residues. In some embodiments, there is one staple. In some embodiments, there are two staples. In some embodiments, there are three staples. In some embodiments, two staples are bonded to the same amino acid residue. In some embodiments, two staples are bonded to the same backbone atom. In some embodiments, two staples are bonded to the same backbone carbon atom. In some embodiments, two staples are bonded to an alpha-carbon atom of an amino acid residue, and each independently bonds to another amino acid residue.

In some embodiments, a staple in an agent, e.g., a staple peptide, are bonded to amino acid residues at positions i and i+3. In some embodiments, a staple in an agent, e.g., a staple peptide, are bonded to amino acid residues at positions i and i+4. In some embodiments, a staple in an agent, e.g., a staple peptide, are bonded to amino acid residues at positions i and i+7. Those skilled in the art appreciate that as used in the art, i, i+3, i+4, i+7, are routinely utilized to indicate relevant positions of amino acid residues. In some embodiments, they may also indicate absolute positions in an agent, e.g., a peptide. In some embodiments, i is an integer of 1-50 (e.g., 1, 2, 3, 4, 5, 6, 7, 8, 9, 10, 11, 12, 13, 14, 15, 16, 17, 18, 19, or 20). In some embodiments, i is 1. In some embodiments, i is 2. In some embodiments, i is 3. In some embodiments, i is 4.

In some embodiments, a provided agent, e.g., a peptide agent such as a stapled peptide agent, comprises one or more (e.g., 1, 2, 3, or 4) of the following groups (in some embodiments, from the N to C direction):
an aromatic group (e.g., of an amino acid residue at position i (e.g., X² as described herein));
an aromatic group (e.g., of an amino acid residue at position i+1 (e.g., X³ as described herein));
a hydrophobic group (e.g., of a side chain of a hydrophobic amino acid at position i+4 (e.g., X⁶ as described herein)); and
a hydrophobic group (e.g., of a side chain of a hydrophobic amino acid at position i+8 (e.g., X¹⁰ as described herein)).
   In some embodiments, a staple is at i+2 and i+9.

In some embodiments, a provided agent, e.g., a peptide agent such as a stapled peptide agent, comprises one or more (e.g., 1, 2, 3, or 4) of the following groups (in some embodiments, from the N to C direction):
an aromatic group (e.g., of an amino acid residue at position i (e.g., X⁴² as described herein);
an acidic group (e.g., of an acidic amino acid residue at position i+1 (e.g., X² as described herein));
an aromatic group (e.g., of an amino acid residue at position i+5 (e.g., X⁶ as described herein)) or a hydrophobic group (e.g., of a side chain of a hydrophobic amino acid at position i+5 (as described herein)); and
a hydrophobic group (e.g., of a side chain of a hydrophobic amino acid at position i+9 (e.g., X¹⁰ as described herein)).
   In some embodiments, a staple is at i+3 and i+10.

In some embodiments, a provided agent, e.g., a peptide agent such as a stapled peptide agent, comprises one or more (e.g., 1, 2, 3, or 4) of the following groups (in some embodiments, from the N to C direction):
an aromatic group (e.g., of an amino acid residue at position i (e.g., X⁴³ as described herein));
a hydrophobic group (e.g., of a side chain of a hydrophobic amino acid at position i+4 (e.g., X⁵ as described herein)).
an acidic group (e.g., of an acidic amino acid residue at position i+5 (e.g., X⁶ as described herein));
a hydrophobic group (e.g., of a side chain of a hydrophobic amino acid at position i+8 (e.g., X⁹ as described herein)).
   In some embodiments, a staple is at i+3 and i+10.

In some embodiments, a provided agent, e.g., a peptide agent such as a stapled peptide agent, comprises one or more (e.g., 1, 2, 3, or 4) of the following groups (in some embodiments, from the N to C direction):
an acidic group (e.g., of an acidic amino acid residue at position i (e.g., X³ as described herein));
an aromatic group (e.g., of an amino acid residue at position i+3 (e.g., X⁶ as described herein));
an aromatic group (e.g., of an amino acid residue at position i+4 (e.g., X⁷ as described herein)); and
a hydrophobic group (e.g., of a side chain of a hydrophobic amino acid at position i+5 (e.g., X⁸ as described herein)).
   In some embodiments, a staple is at i+1 and i+8.

In some embodiments, a provided agent, e.g., a peptide agent such as a stapled peptide agent, comprises one or more (e.g., 1, 2, 3, or 4) of the following groups (in some embodiments, from the N to C direction):
an acidic group (e.g., of an acidic amino acid residue at position i (e.g., X² as described herein));
a hydrophobic group (e.g., of a side chain of a hydrophobic amino acid at position i+1 (e.g., X² as described herein)).
an aromatic group (e.g., of an amino acid residue at position i+4 (e.g., X⁶ as described herein));
a hydrophobic group (e.g., of a side chain of a hydrophobic amino acid at position i+5 (e.g., X⁷ as described herein)).
   In some embodiments, a staple is at i+2 and i+9.

In some embodiments, a provided agent, e.g., a peptide agent such as a stapled peptide agent, comprises one or more (e.g., 1, 2, 3, or 4) of the following groups (in some embodiments, from the N to C direction):
an aromatic group (e.g., of an amino acid residue at position i (e.g., X³³ as described herein));
a hydrophobic group (e.g., of a side chain of a hydrophobic amino acid at position i+4 (e.g., X⁵ as described herein));
a hydrophobic group (e.g., of a side chain of a hydrophobic amino acid at position i+7 (e.g., X⁸ as described herein)); and
an acidic group (e.g., of an acidic amino acid residue at position i+11 (e.g., X¹² as described herein)).
   In some embodiments, a staple is at i+3 and i+10.

In some embodiments, the present disclosure provides an agent which is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, the present disclosure provides an agent which is or comprises a peptide comprising:

[X]ₚX¹X²X³X⁴X⁵X⁶X⁷X⁸X⁹X¹⁰X¹X¹²X¹³X¹⁴[X¹⁵]ₚ₁₅[X¹⁶]ₚ₁₆[X¹⁷]ₚ₁₇[X]_{p′},

wherein:
each of p15, p16 and p17 is independently 0 or 1;
each of p and p′ is independently 0-10;
each of X, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue.

In some embodiments, X¹ is X³¹ as described herein. In some embodiments, X¹ is X³² as described herein. In some embodiments, X¹ is X³³ as described herein. In some embodiments, X¹ is X⁴¹ as described herein. In some embodiments, X¹ is X⁴² as described herein. In some embodiments, X¹ is X⁴³ as described herein.

In some embodiments, an agent is

R^{N}—[X]pX¹X²X³X⁴X⁵X⁶X⁷X⁸X⁹X¹X¹X¹²X¹³[X¹⁴]ₚ₁₄[X¹⁵]ₚ₁₅[X¹⁶]ₚ₁₆[X¹⁷]ₚ₁₇[X]p′—R^{C},

wherein each variable is independently as described herein.

In some embodiments, an agent is or comprises

X¹X²X³X⁴X⁵X⁶X⁷X⁸X⁹X¹⁰X¹¹X¹²X¹³[X¹⁴]ₚ₁₄[X¹⁵]ₚ₁₅[X¹⁶]ₚ₁₆[X¹⁷]ₚ₁₇[X¹⁸]ₚ₁₈[X¹⁹]ₚ₁₉[X²⁰]ₚ₂₀[X²¹]ₚ₂₁[X²²]ₚ₂₂[X²³]_{p 23},

wherein each of p14, p15, p16, p17, p18, p19, p20, p21, p22, and p23 is independently 0 or 1, and each of X¹, X², X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ is independently an amino acid residue as described herein.

In some embodiments, such a peptide comprises residues suitable for stapling. In some embodiments, such a peptide comprises a staple.

In some embodiments, the present disclosure provides an agent, wherein the agent is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein:
each of p0, p15, p16 and p17 is independently 0 or 1;
each of X⁰, X¹, X², X³, X⁴, X⁵, X⁶, X⁷, X⁸, X⁹, X¹⁰, X¹¹, X¹², X¹³, X¹⁴, X¹⁵, X¹⁶, and X¹⁷ is independently an amino acid residue, wherein:
X² comprises a side chain comprising an acidic or a polar group;
X⁵ comprises a side chain comprising an acidic or a polar group;
X¹³ comprises a side chain comprising an optionally substituted aromatic group; and
two or more of X¹, X³, X⁴, X⁷, X¹⁰, X¹¹ and X¹⁴ are each independently an amino acid residue suitable for stapling, or are each independently stapled.

In some embodiments, the present disclosure provides an agent, wherein the agent is or comprises a peptide comprising:

[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇,

wherein each variable is independently as described herein.

In some embodiments, an agent is or comprises a peptide. In some embodiments, an agent is or comprises a stapled peptide. In some embodiments, an agent is a peptide. In some embodiments, an agent is a stapled peptide. In some embodiments, an agent, a peptide, or a stapled peptide comprises X¹X²X³X⁴X⁵X⁶X⁷X⁸X⁹X¹⁰X¹¹X¹²X¹³X¹⁴. In some embodiments, an agent, a peptide, or a stapled peptide comprises X¹X²X³X⁴X⁵X⁶X⁷X⁸X⁹X¹⁰X¹¹X¹²X¹³X¹⁴, wherein X¹ is X³¹, X³², X³³, X⁴¹, X⁴², or X⁴³. In some embodiments, an agent, a peptide, or a stapled peptide has the structure of [X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇. In some embodiments, X⁴ and X¹¹ are stapled.

In some embodiments, the present disclosure provides agents that bind to MDM2 or a fragment thereof. In some embodiments, the present disclosure provides agents that bind to CHIP or a fragment thereof. In some embodiments, an agent, e.g., a peptide, has a molecular mass of no more than about 5000 Daltons. In some embodiments, it is no more than about 2500, 3000, 3500, 4000, 4500 or 5000 Daltons. In some embodiments, it is no more than about 2500 Daltons. In some embodiments, it is no more than about 3000 Daltons. In some embodiments, it is no more than about 3500 Daltons. In some embodiments, it is no more than about 4000 Daltons. In some embodiments, it is no more than about 500 Daltons.

In some embodiments, the present disclosure provides various technologies, e.g., reagents methods, etc., for preparing, characterizing, assessing and using provided agents and compositions thereof. In some embodiments, the present disclosure provides, e.g., methods, reagents and/or systems for identifying, characterizing and/or assessing provided agents and use thereof (e.g., as therapeutic or diagnostic agents).

In some embodiments, the present disclosure provides pharmaceutical compositions comprising or delivering a provided agent and a pharmaceutical acceptable carrier. In some embodiments, a provided agent is a pharmaceutically acceptable salt form. In some embodiments, a provided composition comprises a pharmaceutically acceptable salt form an agent. In some embodiments, in various compositions and methods, agents are provided as pharmaceutically acceptable salt forms.

In some embodiments, the present disclosure provides methods for modulating a property, activity and/or function of MDM2, comprising contacting MDM2 with a provided agent. In some embodiments, the present disclosure provides methods for modulating a property, activity and/or function of MDM2 in a system comprising MDM2, comprising administering or delivering to a system an effective amount of a provided agent. In some embodiments, the present disclosure provides methods for modulating a property, activity and/or function of MDM2 in a system expressing MDM2, comprising administering or delivering to a system an effective amount of a provided agent. In some embodiments, an activity of MDM2 is inhibited or reduced. In some embodiments, a function of MDM2 is inhibited or reduced.

In some embodiments, the present disclosure provides methods for modulating a property, activity and/or function of CHIP, comprising contacting CHIP with a provided agent. In some embodiments, the present disclosure provides methods for modulating a property, activity and/or function of CHIP in a system comprising CHIP, comprising administering or delivering to a system an effective amount of a provided agent. In some embodiments, the present disclosure provides methods for modulating a property, activity and/or function of CHIP in a system expressing CHIP, comprising administering or delivering to a system an effective amount of a provided agent. In some embodiments, an activity of CHIP is inhibited or reduced. In some embodiments, a function of CHIP is inhibited or reduced.

In some embodiments, a system is in vitro. In some embodiments, a system is ex vivo. In some embodiments, a system is in vivo. In some embodiments, a system is or comprise a cell. In some embodiments, a system is or comprises a tissue. In some embodiments, a system is or comprises an organ. In some embodiments, a system is or comprises an organism. In some embodiments, a system is an animal. In some embodiments, a system is human. In some embodiments, a system is or comprises cells, tissues or organs associated with a condition, disorder or disease. In some embodiments, a system is or comprises cancer cells.

In some embodiments, the present disclosure provides methods for preventing conditions, disorders or diseases. In some embodiments, the present disclosure provides methods for reducing risks of conditions, disorders or diseases. In some embodiments, the present disclosure provides methods for preventing a condition, disorder or disease, comprising administering or delivering to a subject susceptible thereto an effective amount of an agent of the present disclosure. In some embodiments, the present disclosure provides methods for reducing risk of a condition, disorder or disease, comprising administering or delivering to a subject susceptible thereto an effective amount of an agent of the present disclosure. In some embodiments, the present disclosure provides methods for reducing risks of a condition, disorder or disease in a population, comprising administering or delivering to a population of subjects susceptible thereto an effective amount of an agent of the present disclosure. In some embodiments, the present disclosure provides methods for treating conditions, disorders or diseases. In some embodiments, the present disclosure provides methods for treating a condition, disorder or disease, comprising administering or delivering to a subject suffering therefrom an effective amount of an agent of the present disclosure. In some embodiments, a symptom is reduced, removed or prevented. In some embodiments, one or more parameters for assessing a condition, disorder or disease are improved. In some embodiments, survival of subjects are extended. As appreciated by those skilled in the art, in some embodiments, prevention, reduced risks, and/or effects of treatment may be assessed through clinical trials and may be observed in subject populations. In some embodiments, a condition, disorder or disease is cancer. In some embodiments, a condition, disorder or disease is associated with MDM2. In some embodiments, a condition, disorder or disease is associated with CHIP. In some embodiments, a condition, disorder or disease is cancer.

In some embodiments, agents are administered as pharmaceutically compositions that comprise or deliver such agents. In some embodiments, agents are provided and/or delivered in pharmaceutically acceptable salt forms. In some embodiments, in a composition (e.g., a liquid composition of certain pH) an agent may exist in various forms including various pharmaceutically acceptable salt forms.

Further description of certain embodiments of provided technologies is presented below.

### DETAILED DESCRIPTION OF CERTAIN EMBODIMENTS

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Affinity: As is known in the art, “affinity” is a measure of the tightness with a particular ligand (e.g., an agent) binds to its partner (e.g., a protein or a portion thereof). Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety. In some embodiments, an agent is a compound. In some embodiments, an agent is a stapled peptide.

Aliphatic: As used herein, “aliphatic” means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Amino acid: In its broadest sense, as used herein, refers to any compound and/or substance that can be incorporated into a polypeptide chain, e.g., through formation of one or more peptide bonds. In some embodiments, an amino acid comprising an amino group and an a carboxylic acid group. In some embodiments, an amino acid has the structure of NH(R^{a1})-L^{a1}-C(R^{a2})(R^{a3})-L^{a2}-COOH, wherein each variable is independently as described in the present disclosure. In some embodiments, an amino acid has the general structure NH(R′)—C(R′)₂—COOH, wherein each R′ is independently as described in the present disclosure. In some embodiments, an amino acid has the general structure H₂N—C(R′)₂—COOH, wherein R′ is as described in the present disclosure. In some embodiments, an amino acid has the general structure H₂N—C(H)(R′)—COOH, wherein R′ is as described in the present disclosure. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy- and/or amino-terminal amino acid in a polypeptide, can contain a structural modification as compared with the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, one or more hydrogens, and/or the hydroxyl group) as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” may be used to refer to a free amino acid; in some embodiments it may be used to refer to an amino acid residue of a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Animal: As used herein refers to any member of the animal kingdom. In some embodiments, “animal” refers to humans, of either sex and at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, insects, and/or worms. In some embodiments, an animal may be a transgenic animal, genetically engineered animal, and/or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Aryl: The term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

Associated with: Two events or entities are “associated” with one another, as that term is used herein, if the presence, level and/or form of one is correlated with that of the other. For example, a particular entity (e.g., nucleic acid (e.g., genomic DNA, transcripts, mRNA, etc.), polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population).

Binding: It will be understood that the term “binding”, as used herein, typically refers to a non-covalent association between or among agents. In many embodiments herein, binding is addressed with respect to particular agents and their target polypeptides. It will be appreciated by those of ordinary skill in the art that such binding may be assessed in any of a variety of contexts. In some embodiments, binding is assessed with respect to a polypeptide, e.g., MDM2, CHIP, etc. In some embodiments, binding is assessed with respect to one or more amino acid residues of a polypeptide. In some embodiments, binding is assessed with respect to one or more amino acid residues corresponding to (e.g., similarly positioned in three dimensional space and/or having certain similar properties and/or functions) those of a polypeptide.

Binding site: The term “binding site”, as used herein, refers to a region of a target polypeptide, formed in three-dimensional space, that includes one or more or all interaction residues of the target polypeptide. In some embodiments, “binding site” may refer to one or more amino acid residues which comprise or are one or more or all interaction amino acid residues of a target polypeptide. As will be understood by those of ordinary skill in the art, a binding site may include residues that are adjacent to one another on a linear chain, and/or that are distal to one another on a linear chain but near to one another in three-dimensional space when a target polypeptide is folded. A binding site may comprise amino acid residues and/or saccharide residues.

Carrier: as used herein, refers to a diluent, adjuvant, excipient, or vehicle with which a composition is administered. In some exemplary embodiments, carriers can include sterile liquids, such as, for example, water and oils, including oils of petroleum, animal, vegetable or synthetic origin, such as, for example, peanut oil, soybean oil, mineral oil, sesame oil and the like. In some embodiments, carriers are or include one or more solid components.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form—e.g., gas, gel, liquid, solid, etc.

Cycloaliphatic: The term “cycloaliphatic,” as used herein, refers to saturated or partially unsaturated aliphatic monocyclic, bicyclic, or polycyclic ring systems having, e.g., from 3 to 30, members, wherein the aliphatic ring system is optionally substituted. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, the cycloalkyl has 3-6 carbons. The terms “cycloaliphatic” may also include aliphatic rings that are fused to one or more aromatic or nonaromatic rings, such as decahydronaphthyl or tetrahydronaphthyl, where a radical or point of attachment is on an aliphatic ring. In some embodiments, a carbocyclic group is bicyclic. In some embodiments, a carbocyclic group is tricyclic. In some embodiments, a carbocyclic group is polycyclic. In some embodiments, “cycloaliphatic” (or “carbocycle” or “cycloalkyl”) refers to a monocyclic C₃-C₁₀, or C₃-C₆ hydrocarbon, or a C₄-C₁₀, or C₈-C₁₀ bicyclic hydrocarbon that is completely saturated or that contains one or more units of unsaturation, but which is not aromatic, or a C₉-C₁₆ tricyclic hydrocarbon that is completely saturated or that contains one or more units of unsaturation, but which is not aromatic.

Derivative: As used herein, the term “derivative” refers to a structural analogue of a reference substance. That is, a “derivative” is a substance that shows significant structural similarity with the reference substance, for example sharing a core or consensus structure, but also differs in certain discrete ways. In some embodiments, a derivative is a substance that can be generated from the reference substance by chemical manipulation. In some embodiments, a derivative is a substance that can be generated through performance of a synthetic process substantially similar to (e.g., sharing a plurality of steps with) one that generates the reference substance.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Engineered: In general, the term “engineered” refers to the aspect of having been manipulated by the hand of man. For example, in some embodiments, a peptide may be considered to be engineered if its amino acid sequence has been selected by man. For example, an engineered agent has an amino acid sequence that was selected based on preferences for corresponding amino acids at particular sites of protein-protein interactions. In many embodiments, provided agents are engineered agents. In some embodiments, engineered agents are peptide agents comprising non-natural amino acid residues, non-natural amino acid sequences, and/or peptide staples. In some embodiments, provided agents comprise or are engineered peptide agents which comprise engineered sequences.

Halogen: The term “halogen” means F, Cl, Br, or I.

Heteroaliphatic: The term “heteroaliphatic” is given its ordinary meaning in the art and refers to aliphatic groups as described herein in which one or more carbon atoms are replaced with one or more heteroatoms (e.g., oxygen, nitrogen, sulfur, silicon, phosphorus, and the like).

Heteroalkyl: The term “heteroalkyl” is given its ordinary meaning in the art and refers to alkyl groups as described herein in which one or more carbon atoms is replaced with a heteroatom (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NW (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

Heterocyclyl: As used herein, the terms “heterocycle,” “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ′NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95% or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution. Typical amino acid categorizations are summarized below (hydrophobicity scale of Kyte and Doolittle, 1982: A simple method for displaying the hydropathic character of a protein. J. Mol. Biol. 157:105-132):

| | 3 Letter | 1 Letter | Side Chain | Side Chain | Hydropathy Index of |
|---|---|---|---|---|---|
| Amino Acid | Code | Code | Polarity | Acidity / Basicity | Kyte and Doolittle |
| Alanine | Ala | A | nonpolar | neutral | 1.8 |
| Arginine | Arg | R | polar | basic | −4.5 |
| Asparagine | Asn | N | polar | neutral | −3.5 |
| Aspartic acid | Asp | D | polar | acidic | −3.5 |
| Cysteine | Cys | C | nonpolar | neutral | 2.5 |
| Glutamic acid | Glu | E | polar | acidic | −3.5 |
| Glutamine | Gln | Q | polar | neutral | −3.5 |
| Glycine | Gly | G | nonpolar | neutral | −0.4 |
| Histidine | His | H | polar | basic | −3.2 |
| Isoleucine | Ile | I | nonpolar | neutral | 4.5 |
| Leucine | Leu | L | nonpolar | neutral | 3.8 |
| Lysine | Lys | K | polar | basic | −3.9 |
| Methionine | Met | M | nonpolar | neutral | 1.9 |
| Phenylalanine | Phe | F | nonpolar | neutral | 2.8 |
| Proline | Pro | P | nonpolar | neutral | −1.6 |
| Serine | Ser | S | polar | neutral | −0.8 |
| Threonine | Thr | T | polar | neutral | −0.7 |
| Tryptophan | Trp | W | nonpolar | neutral | −0.9 |
| Tyrosine | Tyr | Y | polar | neutral | −1.3 |
| Valine | Val | V | nonpolar | neutral | 4.2 |

| Ambiguous Amino Acids | 3-Letter | 1-Letter |
|---|---|---|
| Asparagine or aspartic acid | Asx | B |
| Glutamine or glutamic acid | Glx | Z |
| Leucine or Isoleucine | Xle | J |
| Unspecified or unknown amino acid | Xaa | X |

As will be understood by those skilled in the art, a variety of algorithms are available that permit comparison of sequences in order to determine their degree of homology, including by permitting gaps of designated length in one sequence relative to another when considering which residues “correspond” to one another in different sequences. Calculation of the percent homology between two nucleic acid sequences, for example, can be performed by aligning the two sequences for optimal comparison purposes (e.g., gaps can be introduced in one or both of a first and a second nucleic acid sequences for optimal alignment and non-corresponding sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of the reference sequence. The nucleotides at corresponding nucleotide positions are then compared. When a position in the first sequence is occupied by the same nucleotide as the corresponding position in the second sequence, then the molecules are identical at that position; when a position in the first sequence is occupied by a similar nucleotide as the corresponding position in the second sequence, then the molecules are similar at that position. The percent homology between the two sequences is a function of the number of identical and similar positions shared by the sequences, taking into account the number of gaps, and the length of each gap, which needs to be introduced for optimal alignment of the two sequences. Representative algorithms and computer programs useful in determining the percent homology between two nucleotide sequences include, for example, the algorithm of Meyers and Miller (CABIOS, 1989, 4: 11-17), which has been incorporated into the ALIGN program (version 2.0) using a PAM120 weight residue table, a gap length penalty of 12 and a gap penalty of 4. The percent homology between two nucleotide sequences can, alternatively, be determined for example using the GAP program in the GCG software package using an NWSgapdna.CMP matrix.

Interaction residues: The term “interaction residues”, “interaction motifs”, as used herein, refers to, with respect to an agent, residues or motifs in an agent that are designed to interact with particular target residues in a target polypeptide, or with respect to a target polypeptide, residues in a target polypeptide that interact with particular motifs (e.g., aromatic groups, amino acid residues, etc.) of an agent. Specifically, interaction residues and motifs of various agents are selected and arranged within the agents so that they will be displayed in three dimensional space within a predetermined distance (or volume) of identified target residues (e.g., upon binding, docking or other interaction assays). In many embodiments, interaction residues are direct-binding residues.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Partially unsaturated: As used herein, the term “partially unsaturated” refers to a moiety that includes at least one double or triple bond. The term “partially unsaturated” is intended to encompass groups having multiple sites of unsaturation, but is not intended to include aryl or heteroaryl moieties.

Peptide: The term “peptide” as used herein refers to a polypeptide. In some embodiments, a peptide is a polypeptide that is relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids. In some embodiments, a length is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acids.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; RingeR's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds with bases. Representative alkali or alkaline earth metal salts include salts of sodium, lithium, potassium, calcium, magnesium, and the like. In some embodiments, pharmaceutically acceptable salts are ammonium salts (e.g., —N(R)₃+). In some embodiments, pharmaceutically acceptable salts are sodium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Polypeptide: As used herein refers to any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Prevent or prevention: as used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. June 2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fem), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In some embodiments, suitable mono-protected amines include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. In some embodiments, suitable di-protected amines include amines that are substituted with two substituents independently selected from those described above as mono-protected amines, and further include cyclic imides, such as phthalimide, maleimide, succinimide, and the like. In some embodiments, suitable di-protected amines include pyrroles and the like, 2,2,5,5-tetramethyl-[1,2,5]azadisilolidine and the like, and azide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl. In some embodiments, suitable protected carboxylic acids include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, α-methoxybenzylidene ortho ester, 1-(N,N-dimethylamino)ethylidene derivative, α-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t-butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a phosphorous linkage protecting group is a group attached to the phosphorous linkage (e.g., an internucleotidic linkage) throughout oligonucleotide synthesis. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Specificity: As is known in the art, “specificity” is a measure of the ability of a particular ligand (e.g., an agent) to distinguish its binding partner (e.g., a polypeptide or a portion thereof) from other potential binding partners (e.g., another polypeptide or a portion thereof, or another portion of a polypeptide).

Substitution: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. In some embodiments, example substituents are described below.

Suitable monovalent substituents are halogen; —(CH₂)₀₋₄R^{∘}; —(CH₂)₀₋₄OR^{∘}; —O(CH₂)₀₋₄R, —O—(CH₂)₀₋₄C(O)OR^{∘}; —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)N(R^{∘})₂; —N(R^{∘})C(S)N(R^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)N(R^{∘})₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSi(R^{∘})₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)N(R^{∘})₂; —C(S)N(R^{∘})₂; —C(S)SR^{∘}; —SC(S)SR^{∘}, —(CH₂)₀₋₄OC(O)N(R^{∘})₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂N(R^{∘})₂; —(CH₂)₀₋₄S(O)R^{∘}; —N(R^{∘})S(O)₂N(R^{∘})₂; —N(R^{∘})S(O)₂R^{∘}; —N(OR^{∘})R^{∘}; —C(NH)N(R^{∘})₂; —Si(R^{∘})₃; —OSi(R^{∘})₃; —P(R^{∘})₂; —P(OR^{∘})₂; —OP(R^{∘})₂; —OP(OR^{∘})₂; —N(R^{∘})P(R^{∘})₂; —B(R^{∘})₂; —OB(R^{∘})₂; —P(O)(R^{∘})₂; —OP(O)(R^{∘})₂; —N(R^{∘})P(O)(R^{∘})₂; —(C₁₋₄ straight or branched alkylene)O—N(R^{∘})₂; or —(C₁₋₄ straight or branched alkylene)C(O)O—N(R^{∘})₂; wherein each R^{∘} may be substituted as defined below and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 3-20 (e.g., 3-15, 3-10, 3, 4, 5, 6, 7, 8, 9, 10, 11, 12, 13, 14, 15, 16, 17, 18, 19, 20, etc.) membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 3-20 (e.g., 3-15, 3-10, 3, 4, 5, 6, 7, 8, 9, 10, 11, 12, 13, 14, 15, 16, 17, 18, 19, 20, etc.) membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)_{0- 2}NR^{●}₂, —NO₂, —SiR^{∘}₃, —OSiR^{∘}₃, —C(O)SR, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄aliphatic, —CH₂Ph, —O(CH₂)₀₋₁Ph, or a 3-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents are the following: ═O, ═S, ═NNR*₂, ═NNHC(O)R*, ═NNHC(O)OR*, ═NNHS(O)₂R*, ═NR*, ═NOR*, —O(C(R*₂))₂₃O—, or —S(C(R*₂))₂₋₃S—, wherein each independent occurrence of R* is selected from hydrogen, C₁₋₆ aliphatic which may be substituted as defined below, or an unsubstituted 3-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 3-6-membered saturated, partially unsaturated, or aryl ring having 0-4 heteroatoms independently selected from nitrogen, oxygen, and sulfur.

Suitable substituents on the aliphatic group of R* are halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6-membered saturated, partially unsaturated, or aryl ring having 0-4 heteroatoms independently selected from nitrogen, oxygen, and sulfur.

In some embodiments, suitable substituents on a substitutable nitrogen are —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 3-6-membered saturated, partially unsaturated, or aryl ring having 0-4 heteroatoms independently selected from nitrogen, oxygen, and sulfur, or, notwithstanding the definition above, two independent occurrences of R^{†}, taken together with their intervening atom(s) form an unsubstituted 3-12 (e.g., 3-10, 3-6, 3, 4, 5, 6, 7, 8, 9, 10, etc.) membered saturated, partially unsaturated, or aryl mono- or bicyclic ring having 0-4 heteroatoms independently selected from nitrogen, oxygen, and sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 3-6-membered saturated, partially unsaturated, or aryl ring having 0-4 heteroatoms independently selected from nitrogen, oxygen, and sulfur.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition. In some embodiments, a subject is a human.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Target polypeptide: A “target polypeptide”, as that term is used herein, is a polypeptide with which an agent interacts. In some embodiments, a target polypeptide is a MDM2 or CHIP polypeptide. In some embodiments, a target polypeptide comprises, consists essentially of, or is a binding site of a MDM2 or CHIP polypeptide.

Target residue: A “target residue”, as that term is used herein, is a residue within a target polypeptide with which an agent is designed to interact. For example, an agent may be characterized by particular interaction motifs (e.g., aromatic groups as described herein) and/or residues (e.g., amino acid residues comprising aromatic groups as described herein) selected and arranged (by virtue of being presented on the selected scaffold) to be within a certain predetermined distance (or volume) of a target residue. In some embodiments, a target residue is or comprises an amino acid residue.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutic regimen: A “therapeutic regimen”, as that term is used herein, refers to a dosing regimen whose administration across a relevant population may be correlated with a desired or beneficial therapeutic outcome.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unit dose: The expression “unit dose” as used herein refers to an amount administered as a single dose and/or in a physically discrete unit of a pharmaceutical composition. In many embodiments, a unit dose contains a predetermined quantity of an active agent. In some embodiments, a unit dose contains an entire single dose of the agent. In some embodiments, more than one unit dose is administered to achieve a total single dose. In some embodiments, administration of multiple unit doses is required, or expected to be required, in order to achieve an intended effect. A unit dose may be, for example, a volume of liquid (e.g., an acceptable carrier) containing a predetermined quantity of one or more therapeutic agents, a predetermined amount of one or more therapeutic agents in solid form, a sustained release formulation or drug delivery device containing a predetermined amount of one or more therapeutic agents, etc. It will be appreciated that a unit dose may be present in a formulation that includes any of a variety of components in addition to the therapeutic agent(s). For example, acceptable carriers (e.g., pharmaceutically acceptable carriers), diluents, stabilizers, buffers, preservatives, etc., may be included as described infra. It will be appreciated by those skilled in the art, in many embodiments, a total appropriate daily dosage of a particular therapeutic agent may comprise a portion, or a plurality, of unit doses, and may be decided, for example, by the attending physician within the scope of sound medical judgment. In some embodiments, the specific effective dose level for any particular subject or organism may depend upon a variety of factors including the disorder being treated and the severity of the disorder; activity of specific active compound employed; specific composition employed; age, body weight, general health, sex and diet of the subject; time of administration, and rate of excretion of the specific active compound employed; duration of the treatment; drugs and/or additional therapies used in combination or coincidental with specific compound(s) employed, and like factors well known in the medical arts.

Unsaturated: The term “unsaturated” as used herein, means that a moiety has one or more units of unsaturation.

Unless otherwise specified, salts, such as pharmaceutically acceptable acid or base addition salts, stereoisomeric forms, and tautomeric forms, of provided compound are included.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included.

### Stapled Peptides

In some embodiments, a provided agent is or comprises a peptide. In some embodiments, a provided agent is a peptide. In some embodiments, a peptide is a stapled peptide. In some embodiments, a provided agent is a stapled peptide. In some embodiments, a peptide is a stitched peptide. In some embodiments, a provided agent is a stitched peptide. In some embodiments, a stitched peptide comprises two or more staples, wherein two staples are bonded to the same peptide backbone atom. Stapled peptides as described herein are typically peptides in which two or more amino acids of a peptide chain are linked through connection of two peptide backbone atoms of the amino acid residues and, as is understood by those skilled in the art, the connection is not through the peptide backbone between the linked amino acid residues. In some embodiments, a staple as described herein is a linker that link one amino acid residue to another amino acid residue, e.g., through bonding to a peptide backbone atom of each of the amino acid residues and, as is understood by those skilled in the art, the connection through a staple is not through the peptide backbone between the linked amino acid residues. In some embodiments, a staple bonds to the peptide backbone by replacing one or more hydrogen and/or substituents (e.g., side chains, O, S, etc.) on peptide backbone atoms (e.g., C, N, etc.). In some embodiments, side chains form portions of staples. In some embodiments, a staple is bonded to two carbon backbone atoms, e.g., two alpha carbon atoms. In some embodiments, a staple comprises C(R′)₂ or N(R′), either individually or as part of a large moiety, wherein R′ is R and is taken together with another group attached to a backbone atom which can be R (e.g., R^{a3}) and their intervening atoms to form a ring as described herein (e.g., when PyrS2 is stapled in various peptides).

In some embodiments, a stapled peptide comprises one or more staples. In some embodiments, a stapled peptide comprises two or more staples. In some embodiments, a stapled peptide comprises three or more staples. In some embodiments, a stapled peptide comprises four or more staples. In some embodiments, there are three staples in a stapled peptide. In some embodiments, there are four staples in a stapled peptide.

As will be appreciated by those of ordinary skill in the art, a variety of peptide stapling technologies are available, including both hydrocarbon-stapling and non-hydrocarbon-stapling technologies, and can be utilized in accordance with the present disclosure. Various technologies for stapled and stitched peptides, including various staples and/or methods for manufacturing are available and may be utilized in accordance with the present disclosure, e.g., those described in WO 2019/051327 and WO 2020/041270, the staples of each of which are incorporated herein by reference.

In some embodiments, a peptide, e.g., a stapled peptide, is or comprise a helical structure. In some embodiments, a peptide is a stapled peptide.

In some embodiments, a staple is a hydrocarbon staple. In some embodiments, a staple as described herein is a non-hydrocarbon staple. In some embodiments, a non-hydrocarbon staple comprises one or more chain heteroatoms wherein a chain of a staple is the shortest covalent connection within the staple from one end of the staple to the other end of the staple. In some embodiments, a non-hydrocarbon staple is or comprises at least one sulfur atom derived from an amino acid residue of a polypeptide. In some embodiments, a non-hydrocarbon staple comprises two sulfur atom derived from two different amino acid residues of a polypeptide. In some embodiments, a non-hydrocarbon staple comprises two sulfur atoms derived from two different cysteine residues of a polypeptide. In some embodiments, a staple is a cysteine staple. In some embodiments, a staple is a non-cysteine staple. In some embodiments, a non-hydrocarbon staple is a carbamate staple and comprises a carbamate moiety (e.g., —N(R′)—C(O)—O—) in its chain. In some embodiments, a non-hydrocarbon staple is an amino staple and comprises an amino group (e.g., —N(R′)—) in its chain. In some embodiments, an amino group in an amino staple, e.g., (—N(R′)—) is not bonded to a carbon atom that additionally forms a double bond with a heteroatom (e.g., —C(═O), —C(═S), —C(═N—R′), etc.) so that it is not part of another nitrogen-containing group such as amide, carbamate, etc. In some embodiments, a non-hydrocarbon staple is an ester staple and comprises an ester moiety (—C(O)—O—) in its chain. In some embodiments, a non-hydrocarbon staple is an amide staple and comprises an amide moiety (—C(O)—N(R′)—) in its chain. In some embodiments, a non-hydrocarbon staple is a sulfonamide staple and comprises a sulfonamide moiety (—S(O)₂—N(R′)—) in its chain. In some embodiments, a non-hydrocarbon staple is an ether staple and comprises an ether moiety (—O—) in its chain. In some embodiments, R′ of a carbamate moiety, amino group, amide moiety, sulfonamide moiety, or ether moiety is R, and is taken together with an R group attached to a backbone (e.g., R^{a3} when it is R) and their intervening atoms to form a ring as described herein. In some embodiments, R′ of a carbamate moiety or amino group is R, and is taken together with an R group attached to a backbone (e.g., R^{a3} when it is R) and their intervening atoms to form a ring as described herein.

In some embodiments, a staple comprises one or more amino groups, e.g., —N(R′)—, wherein each R′ is independently as described herein. In some embodiments, —N(R′)— bonds to two carbon atoms. In some embodiments, —N(R′)— bonds to two carbon atoms, wherein neither of the two carbon atoms are bond to any heteroatoms through a double bond. In some embodiments, —N(R′)— bonds to two sp3 carbon atoms. In some embodiments, a staple comprises one or more —C(O)—N(R′)— groups, wherein each R′ is independently as described herein. In some embodiments, a staple comprises one or more carbamate groups, e.g., one or more —(O)—C(O)—N(R′)—, wherein each R′ is independently as described herein. In some embodiments, R′ is —H. In some embodiments, R′ is optionally substituted C₁₋₆ aliphatic. In some embodiments, R′ is optionally substituted C₁₋₆ alkyl. In some embodiments, R′ is C₁₋₆ aliphatic. In some embodiments, R′ is C₁₋₆ alkyl. In some embodiments, R′ is methyl.

In some embodiments, a stapled peptide comprise one or more staples. In some embodiments, a stapled peptide comprises one and no more than one staple. In some embodiments, a stapled peptide comprises two and no more than two staples. In some embodiments, two staples of a stapled peptide bond to a common backbone atom. In some embodiments, two staples of a stapled peptide bond to a common backbone atom which is an alpha carbon atom of an amino acid residue. In some embodiments, a stapled peptide comprises three or more staples. In some embodiments, a stapled peptides comprise four or more staples. In some embodiments, a stapled peptide comprises three and no more than three staples. In some embodiments, a stapled peptide comprises four and no more than four staples. In some embodiments, each staple independently has the structure of -L^{s1}-L^{s2}-L^{s3}- as described herein. In some embodiments, each staple is independently bonded to two amino acid residues. In some embodiments, each staple is independently bonded to two alpha carbon atoms.

In some embodiments, peptides, e.g., staple peptides, of the present disclosure is or comprises a helix structure. As those skilled in the art will appreciate, helixes can have various lengths. In some embodiments, lengths of helixes range from 5 to 30 amino acid residues. In some embodiments, a length of a helix is 5, 6, 7, 8, 9, 10, 11, 12, 13, 14, 15, 16, 17, 18, 19, or 20, or more, amino acid residues. In some embodiments, a length of a helix is 6 amino acid residues. In some embodiments, a length of a helix is 8 amino acid residues. In some embodiments, a length of a helix is 10 amino acid residues. In some embodiments, a length of a helix is 12 amino acid residues. In some embodiments, a length of a helix is 14 amino acid residues. In some embodiments, a length of a helix is 16 amino acid residues. In some embodiments, a length of a helix is 17 amino acid residues. In some embodiments, a length of a helix is 18 amino acid residues. In some embodiments, a length of a helix is 19 amino acid residues. In some embodiments, a length of a helix is 20 amino acid residues.

Amino acids stapled together can have various number of amino acid residues in between, e.g., 1-20, 1-15, 1-10, 1, 2, 3, 4, 5, 6, 7, 8, 9, or 10, etc. In some embodiments, a staple is (i, i+4) which means there are three amino acid residues between the two amino acids (at positions i and i+4, respectively) that bond to the staple (at positions i+1, i+2, i+3, respectively). In some embodiments, a staple is (i, i+2). In some embodiments, a staple is (i, i+3). In some embodiments, a staple is (i, i+5). In some embodiments, a staple is (i, i+6). In some embodiments, a staple is (i, i+7).

In some embodiments, a stapled peptide comprises a staple which staple is L^{s}, wherein L^{s} is -L^{s1}-L^{s2}-L^{s3}-, each of L^{s}, L^{s2}, and L^{s3} is independently L, wherein each L is independently as described in the present disclosure. In some embodiments, a provided staple is L^{s}.

In some embodiments, L^{s1} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s1} is -L′-N(R′)—, wherein L′ is optionally substituted bivalent C₁-C₁₉ aliphatic. In some embodiments, L^{s1} is -L′-N(CH₃)—, wherein L′ is optionally substituted bivalent C₁-C₁₉ aliphatic.

In some embodiments, R′ is optionally substituted C₁₋₆ alkyl. In some embodiments, R′ is C₁₋₆ alkyl. In some embodiments, R′ is methyl. In some embodiments, the peptide backbone atom to which L^{s1} is bonded is also bonded to R¹, and R′ and R¹ are both R and are taken together with their intervene atoms to form an optionally substituted ring as described in the present disclosure. In some embodiments, a formed ring has no additional ring heteroatoms in addition to the nitrogen atom to which R′ is bonded. In some embodiments, a formed ring is 3-membered. In some embodiments, a formed ring is 4-membered. In some embodiments, a formed ring is 5-membered. In some embodiments, a formed ring is 6-membered.

In some embodiments, L′ is optionally substituted bivalent C₁-C₂₀ aliphatic. In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₅ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₄aliphatic. In some embodiments, L′ is optionally substituted alkylene. In some embodiments, L′ is optionally substituted alkenylene. In some embodiments, L′ is unsubstituted alkylene. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—. In some embodiments, L′ is bonded to a peptide backbone atom. In some embodiments, L′ is optionally substituted alkenylene. In some embodiments, L′ is unsubstituted alkenylene. In some embodiments, L′ is —CH₂—CH═CH—CH₂—.

In some embodiments, L′ is optionally substituted phenylene.

In some embodiments, L^{s1} comprises at least one —N(R′)C(O)—, wherein R′ is as described in the present disclosure. In some embodiments, L^{s1} is -L′-N(R′)C(O)—, wherein each of L′ and R′ is independently as described in the present disclosure. In some embodiments, L^{s1} is -L′-N(CH₃)C(O)—, wherein L′ is independently as described in the present disclosure.

In some embodiments, L^{s1} comprises at least one —C(O)O—. In some embodiments, L^{s1} comprises at least one —C(O)O—. In some embodiments, L^{s1} is -L′-C(O)O— or -L′-OC(O)—, wherein each L′ is independently as described in the present disclosure. In some embodiments, L^{s1} is -L′-C(O)O—, wherein each L′ is independently as described in the present disclosure. In some embodiments, L^{s1} is -L′-OC(O)—, wherein each L′ is independently as described in the present disclosure.

In some embodiments, L^{s1} comprises at least one —S(O)₂—N(R′)—, wherein R′ is as described in the present disclosure. In some embodiments, L^{s1} comprises at least one —S(O)₂—N(R′)—, wherein R′ is as described in the present disclosure. In some embodiments, L^{s1} is -L′-N(R′)—S(O)₂— or -L′-S(O)₂—N(R′)— wherein each of L′ and R′ is independently as described in the present disclosure. In some embodiments, L^{s1} is -L′-N(R′)—S(O)₂—, wherein each of L′ and R′ is independently as described in the present disclosure. In some embodiments, L^{s1} is -L′-S(O)₂—N(R′)—, wherein each of L′ and R′ is independently as described in the present disclosure. In some embodiments, L^{s1} is -L′-N(CH₃)—S(O)₂— or -L′-S(O)₂—N(CH₃)—, wherein each L′ is independently as described in the present disclosure. In some embodiments, L^{s1} is -L′-N(CH₃)—S(O)₂—, wherein L′ is as described in the present disclosure. In some embodiments, L^{s1} is -L′-S(O)₂—N(CH₃)—, wherein L′ is as described in the present disclosure.

In some embodiments, L^{s1} comprises at least one —O—. In some embodiments, L^{s1} is -L′-O—, wherein L′ is independently as described in the present disclosure.

In some embodiments, L^{s1} is a covalent bond.

In some embodiments, L^{s1} is L′, wherein L′ is as described in the present disclosure.

In some embodiments, L^{s2} is L, wherein L is as described in the present disclosure. In some embodiments, L^{s2} is L′, wherein L′ is as described in the present disclosure. In some embodiments, L^{s2} comprises —CH₂—CH═CH—CH₂—. In some embodiments, L^{s2} is —CH₂—CH═CH—CH₂—. In some embodiments, L^{s2} comprises —(CH₂)₄—. In some embodiments, L^{s2} is —(CH₂)₄—.

In some embodiments, L^{s3} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s3} is -L′-N(R′)—, wherein L′ is optionally substituted bivalent C₁-C₁₉ aliphatic. In some embodiments, L^{s3} is -L′-N(CH₃)—, wherein L′ is optionally substituted bivalent C₁-C₁₉ aliphatic.

In some embodiments, L^{s3} comprises at least one —N(R′)C(O)—, wherein R′ is as described in the present disclosure. In some embodiments, L^{s3} is -L′-N(R′)C(O)—, wherein each of L′ and R′ is independently as described in the present disclosure. In some embodiments, L^{s3} is -L′-N(CH₃)C(O)—, wherein L′ is independently as described in the present disclosure.

In some embodiments, L^{s3} comprises at least one —C(O)O—. In some embodiments, L^{s3} comprises at least one —C(O)O—. In some embodiments, L^{s3} is -L′—C(O)O— or -L′-OC(O)—, wherein each L′ is independently as described in the present disclosure. In some embodiments, L^{s3} is -L′—C(O)O—, wherein each L′ is independently as described in the present disclosure. In some embodiments, L^{s3} is -L′-OC(O)—, wherein each L′ is independently as described in the present disclosure.

In some embodiments, L^{s3} comprises at least one —S(O)₂—N(R′)—, wherein R′ is as described in the present disclosure. In some embodiments, L^{s3} comprises at least one —S(O)₂—N(R′)—, wherein R′ is as described in the present disclosure. In some embodiments, L^{s3} is -L′-N(R′)—S(O)₂— or -L′-S(O)₂—N(R′)— wherein each of L′ and R′ is independently as described in the present disclosure. In some embodiments, L^{s3} is -L′-N(R′)—S(O)₂—, wherein each of L′ and R′ is independently as described in the present disclosure. In some embodiments, L^{s3} is -L′-S(O)₂—N(R′)—, wherein each of L′ and R′ is independently as described in the present disclosure. In some embodiments, L^{s3} is -L′-N(CH₃)—S(O)₂— or -L′-S(O)₂—N(CH₃)—, wherein each L′ is independently as described in the present disclosure. In some embodiments, L^{s3} is -L′-N(CH₃)—S(O)₂—, wherein L′ is as described in the present disclosure. In some embodiments, L^{s3} is -L′-S(O)₂—N(CH₃)—, wherein L′ is as described in the present disclosure.

In some embodiments, L^{s3} comprises at least one —O—. In some embodiments, L^{s3} is -L′-O—, wherein L′ is independently as described in the present disclosure.

In some embodiments, L^{s3} is L′, wherein L′ is as described in the present disclosure. In some embodiments, L^{s3} is optionally substituted alkylene. In some embodiments, L^{s3} is unsubstituted alkylene.

In some embodiments, L^{s} comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L^{s} comprises at least one —N(R′)C(O)— wherein R′ is as described in the present disclosure.

In some embodiments, L^{s}, L^{s1}, L^{s2}, and L^{s3} each independently and optionally comprise a R′ group, e.g., a R′ group in —C(R′)₂—, —N(R′)—, etc., and the R′ group is taken with a group (e.g., a group that can be R) attached to a backbone atom (e.g., R^{a1}, R^{a2}, R^{a3}, a R′ group of L^{a1} or L^{a2} (e.g., a R′ group in —C(R′)₂—, —N(R′)—, etc.), etc.) to form a double bond or an optionally substituted ring as two R groups can. In some embodiments, a formed ring is an optionally substituted 3-10 membered ring. In some embodiments, a formed ring is an optionally substituted 3-membered ring. In some embodiments, a formed ring is an optionally substituted 4-membered ring. In some embodiments, a formed ring is an optionally substituted 5-membered ring. In some embodiments, a formed ring is an optionally substituted 6-membered ring. In some embodiments, a formed ring is monocyclic. In some embodiments, a formed ring is saturated. In some embodiments, a formed ring is partially unsaturated. In some embodiments, a formed ring is aromatic. In some embodiments, a formed ring comprises one or more ring heteroatom (e.g., nitrogen). In some embodiments, a staple, or L^{s}, L^{s1}, L^{s2}, and/or L^{s3} comprises —N(R′)—, and the R′ is taken together with a group attached to a backbone atom to form an optionally substituted ring as described herein. In some embodiments, a staple, or L^{s}, L^{s1}, L^{s2}, and/or L^{s3} comprises —C(R′)₂—, and the R′ is taken together with a group attached to a backbone atom to form an optionally substituted ring as described herein.

In some embodiments, a staple, or L^{s}, L^{s1}, L^{s2}, and/or L^{s3} comprises portions of one or more amino acid side chains (e.g., a side chain other than its terminal ═CH₂).

As will be clear to those skilled in the art reading the present disclosure, the letter “L” is used to refer to a linker moiety as described herein; each L^{superscript} (e.g., L^{a}, L^{s1}, L^{s2}, L^{s3}, L^{s}, etc.) therefore is understood, in some embodiments, to be L, unless otherwise specified.

In some embodiments, L comprises at least one —N(R′)—, wherein R′ is as described in the present disclosure. In some embodiments, the —N(R′)— is bonded to two carbon atoms, wherein neither of the two carbon atoms forms a double bond with a heteroatom. In some embodiments, the —N(R′)— is not bonded to —C(O)—. In some embodiments, the —N(R′)— is not bonded to —C(S)—. In some embodiments, the —N(R′)— is not bonded to —C(═NR′)—. In some embodiments, L is -L′-N(R′)—, wherein L′ is optionally substituted bivalent C₁-C₁₉ aliphatic. In some embodiments, L is -L′-N(CH₃)—, wherein L′ is optionally substituted bivalent C₁-C₁₉ aliphatic.

In some embodiments, L comprises at least one —N(R′)C(O)—, wherein R′ is as described in the present disclosure. In some embodiments, L is -L′-N(R′)C(O)—, wherein each of L′ and R′ is independently as described in the present disclosure. In some embodiments, L is -L′-N(CH₃)C(O)—, wherein L′ is independently as described in the present disclosure.

In some embodiments, L comprises at least one —C(O)O—. In some embodiments, L comprises at least one —C(O)O—. In some embodiments, L is -L′—C(O)O— or -L′-OC(O)—, wherein each L′ is independently as described in the present disclosure. In some embodiments, L is -L′—C(O)O—, wherein each L′ is independently as described in the present disclosure. In some embodiments, L is -L′-OC(O)—, wherein each L′ is independently as described in the present disclosure.

In some embodiments, L comprises at least one —S(O)₂—N(R′)—, wherein R′ is as described in the present disclosure. In some embodiments, L comprises at least one —S(O)₂—N(R′)—, wherein R′ is as described in the present disclosure. In some embodiments, L is -L′-N(R′)—S(O)₂— or -L′-S(O)₂—N(R′)— wherein each of L′ and R′ is independently as described in the present disclosure. In some embodiments, L is -L′-N(R′)—S(O)₂—, wherein each of L′ and R′ is independently as described in the present disclosure. In some embodiments, L is -L′-S(O)₂—N(R′)—, wherein each of L′ and R′ is independently as described in the present disclosure. In some embodiments, L is -L′-N(CH₃)—S(O)₂— or -L′-S(O)₂—N(CH₃)—, wherein each L′ is independently as described in the present disclosure. In some embodiments, L is -L′-N(CH₃)—S(O)₂—, wherein L′ is as described in the present disclosure. In some embodiments, L is -L′-S(O)₂—N(CH₃)—, wherein L′ is as described in the present disclosure.

In some embodiments, L comprises at least one —O—. In some embodiments, L is -L′-O—, wherein L′ is independently as described in the present disclosure.

In some embodiments, L is L′, wherein L′ is as described in the present disclosure. In some embodiments, L is optionally substituted alkylene. In some embodiments, L is unsubstituted alkylene.

In some embodiments, L is optionally substituted bivalent C₁-C₂₅ aliphatic. In some embodiments, L is optionally substituted bivalent C₁-C₂₀ aliphatic. In some embodiments, L is optionally substituted bivalent C₁-C₁₅ aliphatic. In some embodiments, L is optionally substituted bivalent C₁-C₁₀ aliphatic. In some embodiments, L is optionally substituted bivalent C₁-C₉ aliphatic. In some embodiments, L is optionally substituted bivalent C₁-C₈ aliphatic. In some embodiments, L is optionally substituted bivalent C₁-C₇ aliphatic. In some embodiments, L is optionally substituted bivalent C₁-C₆ aliphatic. In some embodiments, L is optionally substituted bivalent C₁-C₅ aliphatic. In some embodiments, L is optionally substituted bivalent C₁-C₄ aliphatic. In some embodiments, L is optionally substituted alkylene. In some embodiments, L is optionally substituted alkenylene. In some embodiments, L is unsubstituted alkylene. In some embodiments, L is —CH₂—. In some embodiments, L is —(CH₂)₂—. In some embodiments, L is —(CH₂)₃—. In some embodiments, L is —(CH₂)₄—. In some embodiments, L is —(CH₂)₅—. In some embodiments, L is —(CH₂)₆—. In some embodiments, L is —(CH₂)₇—. In some embodiments, L is —(CH₂)₈—. In some embodiments, L is bonded to a peptide backbone atom. In some embodiments, L is optionally substituted alkenylene. In some embodiments, L is unsubstituted alkenylene. In some embodiments, L is —CH₂—CH═CH—CH₂—.

In some embodiments, one end of a staple is connected to an atom Aⁿ¹ of the peptide backbone, wherein Aⁿ¹ is optionally substituted with R¹ and is an atom of an amino acid residue at amino acid position n¹ of the peptide from the N-terminus, and the other end is connected to an atom Aⁿ² of the peptide backbone, wherein Aⁿ² is optionally substituted with R² (in some embodiments, R¹ and/or R² is R which can be hydrogen) and is an atom of an amino acid residue at amino acid position n² of the peptide from the N-terminus, wherein each of n¹ and n² is independently an integer, and n²=n¹+m, wherein m is 3-12.

In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, a staple is referred to a (i, i+m) staple.

In some embodiments, Aⁿ¹ is a carbon atom. In some embodiments, Aⁿ¹ is achiral. In some embodiments, Aⁿ¹ is chiral. In some embodiments, Aⁿ¹ is R. In some embodiments, Aⁿ¹ is S.

In some embodiments, Aⁿ² is a carbon atom. In some embodiments, Aⁿ² is achiral. In some embodiments, Aⁿ² is chiral. In some embodiments, Aⁿ² is R. In some embodiments, Aⁿ² is S.

In some embodiments, Aⁿ¹ is achiral and Aⁿ² is achiral. In some embodiments, Aⁿ¹ is achiral and Aⁿ² is R. In some embodiments, Aⁿ¹ is achiral and Aⁿ² is S. In some embodiments, Aⁿ¹ is R and Aⁿ² is achiral. In some embodiments, Aⁿ¹ is Rand Aⁿ² is R. In some embodiments, Aⁿ¹ is Rand Aⁿ² is S. In some embodiments, Aⁿ¹ is S and Aⁿ² is achiral. In some embodiments, Aⁿ¹ is S and Aⁿ² is R. In some embodiments, Aⁿ¹ is S and Aⁿ² is S.

In some embodiments, provided stereochemistry at staple-backbone connection points and/or combinations thereof, optionally together with one or more structural elements of provided peptide, e.g., staple chemistry (hydrocarbon, non-hydrocarbon), staple length, etc. can provide various benefits, such as improved preparation yield, purity, and/or selectivity, improved properties (e.g., improved solubility, improved stability, lowered toxicity, improved selectivity, etc.), improved activities, etc. In some embodiments, provided stereochemistry and/or stereochemistry combinations are different from those typically used, e.g., those of U.S. Pat. No. 9,617,309, US 2015-0225471, US 2016-0024153, US 2016-0215036, US 2016-0244494, WO 2017/062518, and provided one or more of benefits described in the present disclosure.

In some embodiments, a staple can be of various lengths, in some embodiments, as represent by the number of chain atoms of a staple. In some embodiments, a chain of a staple is the shortest covalent connection in the staple from a first end (connection point with a peptide backbone) of a staple to a second end of the staple, wherein the first end and the second end are connected to two different peptide backbone atoms. In some embodiments, a staple comprises 5-30 chain atoms, e.g., 5-20, 5-15, 5, 6, 7, 8, 9, or 10 to 11, 12, 13, 14, 15, 16, 17, 18, 19, 20, 21, 22, 23, 24, or 25 chain atoms. In some embodiments, a staple comprises 5 chain atoms. In some embodiments, a staple comprises 6 chain atoms. In some embodiments, a staple comprises 7 chain atoms. In some embodiments, a staple comprises 8 chain atoms. In some embodiments, a staple comprises 9 chain atoms. In some embodiments, a staple comprises 10 chain atoms. In some embodiments, a staple comprises 11 chain atoms. In some embodiments, a staple comprises 12 chain atoms. In some embodiments, a staple comprises 13 chain atoms. In some embodiments, a staple comprises 14 chain atoms. In some embodiments, a staple comprises 15 chain atoms. In some embodiments, a staple comprises 16 chain atoms. In some embodiments, a staple comprises 17 chain atoms. In some embodiments, a staple comprises 18 chain atoms. In some embodiments, a staple comprises 19 chain atoms. In some embodiments, a staple comprises 20 chain atoms. In some embodiments, a staple has a length of 5 chain atoms. In some embodiments, a staple has a length of 6 chain atoms. In some embodiments, a staple has a length of 7 chain atoms. In some embodiments, a staple has a length of 8 chain atoms. In some embodiments, a staple has a length of 9 chain atoms. In some embodiments, a staple has a length of 10 chain atoms. In some embodiments, a staple has a length of 11 chain atoms. In some embodiments, a staple has a length of 12 chain atoms. In some embodiments, a staple has a length of 13 chain atoms. In some embodiments, a staple has a length of 14 chain atoms. In some embodiments, a staple has a length of 15 chain atoms. In some embodiments, a staple has a length of 16 chain atoms. In some embodiments, a staple has a length of 17 chain atoms. In some embodiments, a staple has a length of 18 chain atoms. In some embodiments, a staple has a length of 19 chain atoms. In some embodiments, a staple has a length of 20 chain atoms. In some embodiments, a staple has a length of 8-15 chain atoms. In some embodiments, a staple has 8-12 chain atoms. In some embodiments, a staple has 9-12 chain atoms. In some embodiments, a staple has 9-10 chain atoms. In some embodiments, a staple has 8-10 chain atoms. In some embodiments, length of a staple can be adjusted according to the distance of the amino acid residues it connects, for example, a longer staple may be utilized for a (i, i+7) staple than a (i, i+4) or (i, i+3) staple. In some embodiments, a (i, i+2) staple has about 5-10, 5-8, e.g., about 5, 6, 7, 8, 9 or 10 chain atoms. In some embodiments, a (i, i+2) staple has 5 chain atoms. In some embodiments, a (i, i+2) staple has 6 chain atoms. In some embodiments, a (i, i+2) staple has 7 chain atoms. In some embodiments, a (i, i+2) staple has 8 chain atoms. In some embodiments, a (i, i+2) staple has 9 chain atoms. In some embodiments, a (i, i+2) staple has 10 chain atoms. In some embodiments, a (i, i+3) staple has about 5-10, 5-8, e.g., about 5, 6, 7, 8, 9 or 10 chain atoms. In some embodiments, a (i, i+3) staple has 5 chain atoms. In some embodiments, a (i, i+3) staple has 6 chain atoms. In some embodiments, a (i, i+3) staple has 7 chain atoms. In some embodiments, a (i, i+3) staple has 8 chain atoms. In some embodiments, a (i, i+3) staple has 9 chain atoms. In some embodiments, a (i, i+3) staple has 10 chain atoms. In some embodiments, a (i, i+4) staple has about 5-12, 5-10, 7-12, 5-8, e.g., about 5, 6, 7, 8, 9, 10, 11 or 12 chain atoms. In some embodiments, a (i, i+4) staple has 5 chain atoms. In some embodiments, a (i, i+4) staple has 6 chain atoms. In some embodiments, a (i, i+4) staple has 7 chain atoms. In some embodiments, a (i, i+4) staple has 8 chain atoms. In some embodiments, a (i, i+4) staple has 9 chain atoms. In some embodiments, a (i, i+4) staple has 10 chain atoms. In some embodiments, a (i, i+4) staple has 11 chain atoms. In some embodiments, a (i, i+4) staple has 12 chain atoms. In some embodiments, a (i, i+7) staple has about 8-25, 10-25, 10-16, 12-15, e.g., about 8, 9, 10, 11, 12, 13, 14, 15, 16, 17, 18, 19, 20, 21, 22, 23, 24 or 25 chain atoms. In some embodiments, a (i, i+7) staple has 8 chain atoms. In some embodiments, a (i, i+7) staple has 9 chain atoms. In some embodiments, a (i, i+7) staple has 10 chain atoms. In some embodiments, a (i, i+7) staple has 11 chain atoms. In some embodiments, a (i, i+7) staple has 12 chain atoms. In some embodiments, a (i, i+7) staple has 13 chain atoms. In some embodiments, a (i, i+7) staple has 14 chain atoms. In some embodiments, a (i, i+7) staple has 15 chain atoms. In some embodiments, a (i, i+7) staple has 16 chain atoms. In some embodiments, a (i, i+7) staple has 17 chain atoms. In some embodiments, a (i, i+7) staple has 18 chain atoms. In some embodiments, a (i, i+7) staple has 19 chain atoms. In some embodiments, a (i, i+7) staple has 20 chain atoms. In some embodiments, a (i, i+7) staple has 21 chain atoms. In some embodiments, a (i, i+7) staple has 22 chain atoms. In some embodiments, a stapled peptide comprises three or more staples, each of which is independently such a (I, i+2), (i, i+3), (i, i+4) or (i, i+7) staple. In some embodiments, a stapled peptide comprises such a (i, i+2) staple, such a (i, i+4) staple and such a (i, i+7) staple. In some embodiments, a stapled peptide comprises such a (i, i+3) staple, such a (i, i+4) staple and such a (i, i+7) staple. In some embodiments, a stapled peptide comprises such a (i, i+3) staple, such a (i, i+7) staple and such a (i, i+7) staple.

Staple lengths may be otherwise described. For example, in some embodiments, staple lengths may be described as the total number of chain atoms and non-chain ring atoms, where a non-chain ring atom is an atom of the staple which forms a ring with one or more chain atoms but is not a chain atom in that it is not within the shortest covalent connection from a first end of the staple to a second end of the staple. In some embodiments, staples formed using Monomer A (which comprises an azetidine moiety), Monomer B (which comprises a pyrrolidine moiety), and/or Monomer C (which comprises a pyrrolidine moiety), etc., may comprise one or two non-chain ring atoms.

In some embodiments, a staple has no heteroatoms in its chain. In some embodiments, a staple comprises at least one heteroatom in its chain. In some embodiments, a staple comprises at least one nitrogen atom in its chain.

In some embodiments, a staple is L^{s}, wherein L^{s} is an optionally substituted, bivalent C₈₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₉₋₁₃ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₀₋₁₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is L^{s}, wherein L^{s} is an optionally substituted, bivalent C₁₁₋₁₄ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, a staple is a (i, i+2) staple in that not including the two amino acid residues that are directly connected to the staple, there are one amino acid residue between the two amino acid residues that are directly connected to the staple. In some embodiments, a staple is a (i, i+3) staple in that not including the two amino acid residues that are directly connected to the staple, there are two amino acid residues between the two amino acid residues that are directly connected to the staple. In some embodiments, a staple is a (i, i+4) staple in that not including the two amino acid residues that are directly connected to the staple, there are three amino acid residues between the two amino acid residues that are directly connected to the staple. In some embodiments, a staple is a (i, i+7) staple in that not including the two amino acid residues that are directly connected to the staple, there are six amino acid residues between the two amino acid residues that are directly connected to the staple.

In some embodiments, for each of L^{s}, L^{s1}, L^{s2}, and L^{s3}, any replacement of methylene units, if any, is replaced with —N(R′)—, —C(O)—N(R′)—, —N(R′)C(O)O—, —C(O)O—, —S(O)₂N(R′)—, or —O—. In some embodiments, for each of L^{s}, L^{s1}, L^{s2}, and L^{s3}, any replacement of methylene units, if any, is replaced with —N(R′)—, —N(R′)—C(O)—, or —N(R′)C(O)O—. In some embodiments, for each of L^{s}, L^{s1}, L^{s2}, and L^{s3}, any replacement of methylene units, if any, is replaced with —N(R′)— or —N(R′)C(O)O—. In some embodiments, for each of L^{s}, L^{s1}, L^{s2}, and L^{s3}, any replacement of methylene units, if any, is replaced with —N(R′)—. In some embodiments, for each of L^{s}, L^{s1}, L^{s2}, and L^{s3}, any replacement of methylene units, if any, is replaced with —N(R′)C(O)O—.

In some embodiments, a staple comprises a double bond. In some embodiments, a staple comprises a double bond may be formed by olefin metathesis of two olefins. In some embodiments, staples are formed by metathesis reactions, e.g., involving one or more double bonds in amino acid residues as described herein. In some embodiments, a first amino acid residue comprising an olefin (e.g., AA1-CH═CH₂) and a second amino acid residue comprising an olefin (e.g., AA2-CH═CH₂) are stapled (e.g., forming AA1-CH═CH-AA2, wherein AA1 and AA2 are typically linked through one or more amino acid residues). In some embodiments, an olefin, e.g., in a staple, is converted into —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—. In some embodiments, each of the two olefins is independently of a side chain of an amino acid residue. In some embodiments, each olefin is independently a terminal olefin. In some embodiments, each olefin is independently a mono-substituted olefin.

In some embodiments, an amino acid of formula A-1 or a salt thereof is a compound having the structure of formula A-IL:

NH(R^{a1})-L^{a1}-C(-L^{a}-CH═CH₂)(R^{a3})-L^{a2}-COOH, A-II

or a salt thereof, wherein each variable is independently as described in the present disclosure. In some embodiments, an amino acid suitable for stapling has the structure of formula A-II or a salt thereof, wherein each variable is independently as described in the present disclosure.

In some embodiments, an amino acid of formula A-II or a salt thereof is a compound having the structure of formula A-II-b:

NH(R^{a1})—C(-L^{a}-CH═CH₂)(R^{a3})—COOH, A-II-b

or a salt thereof, wherein each variable is independently as described in the present disclosure. In some embodiments, an amino acid suitable for stapling has the structure of formula A-II-b or a salt thereof, wherein each variable is independently as described in the present disclosure.

In some embodiments, an amino acid of formula A-I or a salt thereof is a compound having the structure of formula A-III:

N(-L^{a}-CH═CH₂)(R^{a1})-L^{a}-C—(-L^{a}-CH═CH₂)(R^{a3})-L^{a2}-COH, A-III

or a salt thereof, wherein each variable is independently as described in the present disclosure. In some embodiments, an amino acid suitable for stapling has the structure of formula A-II or a salt thereof, wherein each variable is independently as described in the present disclosure.

In some embodiments, an amino acid of formula A-I or a salt thereof has structure of formula A-IV:

NH(R^{a1})-L^{a1}-C(-L^{a}-COOH)(R^{a3})-L^{a2}-COOH, A-IV

or a salt thereof, wherein each variable is independently as described in the present disclosure. In some embodiments, an amino acid suitable for stapling has the structure of formula A-IV or a salt thereof, wherein each variable is independently as described in the present disclosure.

In some embodiments, an amino acid has structure of formula A-V:

NH(R^{a1})-L^{a1}-C(-L^{a}-R^{SP1})(R^{a3})-L^{a2}COOH, A-V

or a salt thereof, wherein each variable is independently as described in the present disclosure. In some embodiments, an amino acid suitable for stapling has the structure of formula A-V or a salt thereof, wherein each variable is independently as described in the present disclosure.

In some embodiments, an amino acid for stapling has structure of formula A-VI:

NH(R^{a1})-L^{a1}-C(-L^{a}-R^{SP1})(-L^{a}-R^{SP2})-L^{a2}-COOH, A-VI

or a salt thereof, wherein each variable is independently as described in the present disclosure. In some embodiments, an amino acid suitable for stapling has the structure of formula A-VI or a salt thereof, wherein each variable is independently as described in the present disclosure.

As used herein, each of R^{SP1} and R^{SP2} independently comprises a reactive group. In some embodiments, each of R^{SP1} and R^{SP2} is independently a reactive group. In some embodiments, a reactive group is optionally substituted —CH═CH₂. In some embodiments, a reactive group is —CH═CH₂. In some embodiments, a reactive group is an amino group, e.g., —NHR, wherein R is as described herein. In some embodiments, a reactive group is an acid group. In some embodiments, a reactive group is —COOH or an activated form thereof. In some embodiments, a reactive group is for a cycloaddition reaction (e.g., [3+2], [4+2], etc.), e.g., an alkene, an alkyne, a diene, a 1,3-dipole (e.g., —N₃), etc. In some embodiments, a reactive group is optionally substituted —C≡CH. In some embodiments, a reactive group is —C≡CH. In some embodiments, a reactive group is —N₃.

In some embodiments, R^{SP1} or R^{SP2} of a first amino acid residue and R^{SP1} or R^{SP2} of a second amino acid residue can react with each other so that the two amino acid residues are connected with a staple. In some embodiments, a reactive is olefin metathesis between two olefin, e.g., two —CH═CH₂. In some embodiments, a reaction is amidation and one reactive group is an amino group, e.g., —NHR wherein R is as described herein (e.g., in some embodiments, R is —H; in some embodiments, R is optionally substituted C₁₋₆ aliphatic), and the other is an acid group (e.g., —COOH) or an activated form thereof. In some embodiments, a reaction is a cycloaddition reaction, e.g., [4+2], [3+2], etc. In some embodiments, a first and a second reactive groups are two reactive groups suitable for a cycloaddition reaction. In some embodiments, a reaction is a click reaction. In some embodiments, one reaction group is or comprises —N₃, and the other is or comprises an alkyne, e.g., a terminal alkyne or a activated/strained alkyne. In some embodiments, the other is or comprises —C≡CH.

In some embodiments, R^{SP1} or R^{SP2} of a first amino acid residue and R^{SP1} or R^{SP2} of a second amino acid residue can react with a reagent so that the two are connected to form a staple. In some embodiments, a reagent comprises two reactive groups, one of which reacts with R^{SP1} or R^{SP2} of a first amino acid residue, and the other reacts with R^{SP1} or R^{SP2} of a first amino acid residue. In some embodiments, R^{SP1} or R^{SP2} of both amino acid residues are the same or the same type, e.g., both are amino groups, and the two reactive groups of a linking reagent are also the same, e.g., both are acid groups such as —COOH or activated form thereof. In some embodiments, R^{SP1} or R^{SP2} of both amino acid residues are both acid groups, e.g., —COOH or activated form thereof, and both reactive groups of a linking agent are amino groups. In some embodiments, R^{SP1} or R^{SP2} of both amino acid residues are both nucleophilic groups, e.g., —SH, and both reactive groups of a linking reagent are electrophilic (e.g., carbon attached to leaving groups such as —Br, —I, etc.).

In some embodiments, R^{SP1} and R^{SP2} are the same. In some embodiments, R^{SP1} and R^{SP2} are different. In some embodiments, R^{SP1} is or comprises —CH═CH₂. In some embodiments, R^{SP1} is or comprises —COOH. In some embodiments, R^{SP1} is or comprises an amino group. In some embodiments, R^{SP1} is or comprises —NHR. In some embodiments, R is hydrogen or optionally substituted C₁₋₆ aliphatic. In some embodiments, R^{SP1} is or comprises —NH₂. In some embodiments, R^{SP1} is or comprises —N₃. In some embodiments, R^{SP2} is or comprises —CH═CH₂. In some embodiments, R^{SP2} is or comprises —COOH. In some embodiments, R^{SP2} is or comprises an amino group. In some embodiments, R^{SP2} is or comprises —NHR. In some embodiments, R is hydrogen or optionally substituted C₁₋₆ aliphatic. In some embodiments, R^{SP2} is or comprises —NH₂. In some embodiments, R^{SP2} is or comprises —N₃.

In some embodiments, each amino acid residue of a pair of amino acid residues is independently a residue of an amino acid of formula A-II or A-III or a salt thereof. In some embodiments, such a pair of amino acid residues is stapled, e.g., through olefin metathesis. In some embodiments, a staple has the structure of -L^{a}-CH═CH-L^{a}-, wherein each variable is independently as described herein. In some embodiments, olefin in a staple is reduced. In some embodiments, In some embodiments, a staple has the structure of -L^{a}-CH₂—CH₂-L^{a}-, wherein each variable is independently as described herein. In some embodiments, one L^{a} is L^{s1} as described herein, and one L^{a} is L^{s3} as described herein.

In some embodiments, two amino acid residues, e.g., of amino acids independently of formula A-I or a salt of, connected by a staple have the structure of —N(R^{a1}-L^{a1}-C(-L^{s}-R^{AA})(R^{a3})-L^{a2}-CO—, wherein each variable is independently as described herein, and R^{AA} is an amino acid residue. In some embodiments, two amino acid residues, e.g., of amino acids independently of formula A-I or a salt of, connected by a staple have the structure of —N(-L^{s}-R^{AA})-L^{a1}-C(R^{a2})(R^{a3})-L^{a2}-CO—, wherein each variable is independently as described herein, and R^{AA} is an amino acid residue. In some embodiments, two amino acid residues, e.g., of amino acids independently of formula A-I or a salt of, connected by a staple have the structure of R^{a1}N(-L^{s}-R^{AA})-L^{a1}-C(R^{a2})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples have the structure of R^{a1}N(-L^{s}-R^{AA})-L^{a1}-C(-L^{s}-R^{AA})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples have the structure of —N(-L^{s}-R^{AA})-L^{a1}-C(-L^{s}-R^{AA})(R^{a3})-L^{a2}-CO—, wherein each variable is independently as described herein, and R^{AA} is an amino acid residue. In some embodiments, three amino acid residues, e.g., of amino acids independently of formula A-I or a salt of, connected by two staples (e.g., X⁴ stapled with both X¹ and X¹⁴) have the structure of —N(R^{a1}-L^{a1}-C(-L^{s}-R^{AA})(-L^{s}-R^{AA})-L^{a2}-CO—, wherein each variable is independently as described herein, and R^{AA} is an amino acid residue. In some embodiments, each R^{AA} is independently a residue of an amino acid of formula A-I, A-II, A-III, A-IV, A-V, A-VI, etc. or a salt thereof. In some embodiments, R^{AA} is —C(R^{a3})[-L^{a1}N(R^{a1})—](-L^{a2}-CO—), wherein each variable is independently as described herein. In some embodiments, R^{AA} is —C(R^{a3})[—N(R^{a1})—](—CO—), wherein each variable is independently as described herein. In some embodiments, each R^{AA} is independently —N(−)[-L^{a1}-C(R^{a2})(R^{a3})-L^{a2}-CO—], wherein each variable is independently as described herein, wherein —C(−)(R^{a3})— is bonded to a staple. In some embodiments, each R^{AA} is independently —N(−)[—C(R^{a2})(R^{a3})—CO—], wherein each variable is independently as described herein, wherein —C(−)(R^{a3})— is bonded to a staple. In some embodiments, each R^{AA} is independently R^{a1}N(−)[-L^{a1}-C(R^{a2})(R^{a3})-L^{a2}-CO—], wherein each variable is independently as described herein, wherein —C(−)(R^{a3})— is bonded to a staple. In some embodiments, each R^{AA} is independently R^{a1}—N(−)[—C(R^{a2})(R^{a3})—CO—], wherein each variable is independently as described herein, wherein —C(−)(R^{a3})— is bonded to a staple.

Various staples, e.g., L^{s}, are as described herein. In some embodiments, L^{s} is -L^{s1}-L^{s2}-L^{s3}- as described herein. In some embodiments, L^{s1} is L^{a} as described herein. In some embodiments, L^{s3} is L^{a} as described herein. In some embodiments, L^{s1} is L^{a} of a first of two stapled amino acid residues. In some embodiments, L^{s2} is L^{a} of a second of two stapled amino acid residues. In some embodiments, L^{s2} is or comprises a double bond. In some embodiments, L^{s2} is or comprises —CH═CH—. In some embodiments, L^{s2} is or comprises optionally substituted —CH₂—CH₂—. In some embodiments, L^{s2} is or comprises —CH₂—CH₂—. In some embodiments, L^{s2} is or comprises —C(O)N(R′)— (e.g., a staple formed by two amino acid residues one of which has a R^{SP1} group that is or comprises an amino group and the other of which has a R^{SP2} group that is or comprises —COOH). In some embodiments, L^{s2} is or comprises —C(O)NH—. In some embodiments, each of L^{s1} and L^{s3} is independently optionally substituted linear or branched C₁₋₁₀ hydrocarbon chain. In some embodiments, each of L^{s1} and L^{s3} is independently —(CH₂)n-, wherein n is 1-10. In some embodiments, L^{s1} is —CH₂—. In some embodiments, L^{s3} is —(CH₂)₃—.

In some embodiments, L^{s} is —CH₂—CH═CH—(CH₂)₃—. In some embodiments, L^{s} is —(CH₂)₆—.

In some embodiments, L^{s} is —(CH₂)₂—C(O)NH—(CH₂)₄—.

In some embodiments, L^{s} is bonded to two backbone carbon atoms. In some embodiments, L^{s} is bonded to two alpha carbon atoms of two stapled amino acid residues. In some embodiments, L^{s} is bonded to a backbone nitrogen atom and a backbone carbon atom (e.g., an alpha carbon).

In some embodiments, L^{a} comprises at least one —N(R′)— wherein R′ is independently as described in the present disclosure. In some embodiments, L^{a} comprises -L^{am1}-N(R′)— wherein R′ is independently as described in the present disclosure, and L^{am1} is as described herein. In some embodiments, L^{a} is or comprises -L^{am1}-N(R′)-L^{am2}-, wherein each of L^{am1}, R′, and L^{am2} is independently as described herein. In some embodiments, R′ is optionally substituted C₁₋₆ aliphatic. In some embodiments, R′ is methyl. In some embodiments, R′ is taken together with R^{a3} to form an optionally substituted ring as described herein. In some embodiments, a formed ring is a 3-10 membered monocyclic saturated ring as described herein. In some embodiments, a formed ring has no additional heteroatom ring atom in addition to the nitrogen of —N(R′)—. In some embodiments, a formed ring is 3-membered. In some embodiments, a formed ring is 4-membered. In some embodiments, a formed ring is 5-membered. In some embodiments, a formed ring is 6-membered.

In some embodiments, L^{a} comprises at least one —C(R′)₂— wherein each R′ is independently as described in the present disclosure. In some embodiments, L^{a} comprises -L^{am1}-C(R′)₂— wherein R′ is independently as described in the present disclosure, and L^{am1} is as described herein. In some embodiments, L^{a} is or comprises -L^{am1}-C(R′)₂-L^{am2}-, wherein each of L^{am1}, R′, and L^{am2} is independently as described herein. In some embodiments, R′ is —H. In some embodiments, —C(R′)₂— is optionally substituted —CH₂—. In some embodiments, —C(R′)₂— is —CH₂—. In some embodiments, one R′ is taken together with R^{a3} to form an optionally substituted ring as described herein. In some embodiments, a formed ring is a 3-10 membered monocyclic saturated ring as described herein. In some embodiments, a formed ring has no additional heteroatom ring atom in addition to the nitrogen of —N(R′)—. In some embodiments, a formed ring is 3-membered. In some embodiments, a formed ring is 4-membered. In some embodiments, a formed ring is 5-membered. In some embodiments, a formed ring is 6-membered.

As described herein, each of L^{am1} and L^{am2} is independently L^{am} as described herein. As described herein, L^{am} is a covalent bond, or an optionally substituted, bivalent C₁-C₁₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{am} is a covalent bond. In some embodiments, L^{am} is an optionally substituted bivalent C₁-C₁₀ aliphatic group. In some embodiments, L^{am} is an optionally substituted bivalent linear C₁-C₁₀ aliphatic group. In some embodiments, L^{am} is optionally substituted C₁₋₁₀ alkylene. In some embodiments, L^{am} is C₁₋₁₀ alkylene. In some embodiments, L^{am} is optionally substituted linear C₁₋₁₀ alkylene. In some embodiments, L^{am} is optionally substituted —CH₂—. In some embodiments, L^{am} is —CH₂—.

In some embodiments, L^{am1} is a covalent bond. In some embodiments, L^{am1} is an optionally substituted bivalent C₁-C₁₀ aliphatic group. In some embodiments, L^{am1} is an optionally substituted bivalent linear C₁-C₁₀ aliphatic group. In some embodiments, L^{am1} is optionally substituted C₁₋₁₀ alkylene. In some embodiments, L^{am1} is C₁₋₁₀ alkylene. In some embodiments, L^{am1} is optionally substituted linear C₁₋₁₀ alkylene. In some embodiments, L^{am1} is optionally substituted —CH₂—. In some embodiments, L^{am1} is —CH₂—. In some embodiments, L^{am1} is bonded to a backbone atom. In some embodiments, L^{am1} is bonded to an alpha-carbon of an amino acid.

In some embodiments, L^{am2} is a covalent bond. In some embodiments, L^{am2} is an optionally substituted bivalent C₁-C₁₀ aliphatic group. In some embodiments, L^{am2} is an optionally substituted bivalent linear C₁-C₁₀ aliphatic group. In some embodiments, L^{am2} is optionally substituted C₁₋₁₀ alkylene. In some embodiments, L^{am2} is C₁₋₁₀ alkylene. In some embodiments, L^{am2} is optionally substituted linear C₁₋₁₀ alkylene. In some embodiments, L^{am2} is optionally substituted —CH₂—. In some embodiments, L^{am2} is —CH₂—. In some embodiments, L^{am2} is or comprises —C(O)—. In some embodiments, —C(O)— is bonded to a nitrogen atom. In some embodiments, L^{am2} is or comprises —S(O)₂—. In some embodiments, —S(O)₂— is bonded to a nitrogen atom. In some embodiments, L^{am2} is or comprises —O—. In some embodiments, L^{am2} is or comprises —C(O)—O—. In some embodiments, —C(O)—O— is bonded to a nitrogen atom. In some embodiments, L^{am2} is bonded to a nitrogen atom, and it comprises a —C(O)— group which is bonded to the nitrogen atom. In some embodiments, L^{am2} is bonded to a nitrogen atom, and it comprises a —C(O)—O— group which is bonded to the nitrogen atom. In some embodiments, L^{am2} is or comprises —C(O)—O—CH₂—, wherein the —CH₂— is optionally substituted. In some embodiments, L^{am2} is —C(O)—O—CH₂—.

In some embodiments, L^{a} is L^{s1} as described herein. In some embodiments, L^{a} is L^{s2} as described herein.

In some embodiments, R^{a3} is -L^{a}-CH═CH₂, wherein L^{a} is independently as described herein. In some embodiments, each of R^{a2} and R^{a3} independently comprises a double bond, e.g., a terminal olefin which can be optionally and independently stapled with another residue comprising an olefin. In some embodiments, each of R^{a2} and R^{a3} are independently -L^{a}-CH═CH₂. In some embodiments, an amino acid are stapled with two amino acid residues independently through R^{a2} and R^{a3}. In some embodiments, such an amino acid is B5. In some embodiments, it is B3. In some embodiments, it is B4. In some embodiments, it is B6.

In some embodiments, an amino acid is selected from Tables A-I, A-II, and A-III (may be presented as Fmoc-protected). As appreciated by those skilled in the art, among other things, when incorporated into peptides, Fmoc-protected amino groups and carboxyl groups may independently form amide connections with other amino acid residues (or N- or C-terminus capping groups, or exist as N- or C-terminus amino or carboxyl groups). Olefins, including those in Alloc groups, may be utilized to form staples through olefin metathesis. Staples comprising olefins may be further modified, e.g., through hydrogenation to convert olefin double bonds into single bonds, and/or through CO₂ extrusion to convert carbamate moieties (e.g., —O—(CO)—N(R′)—) into amine moieties (e.g., —N(R′)—). In some embodiments, an agent is or comprises a stapled peptide or a salt thereof, in which stapled peptide each double bond is converted into a single bond. In some embodiments, a conversion is achieved through hydrogenation which adds a —H to each olefin carbon atom. In some embodiments, an olefin double bond is replaced with —CHR′—CHR′—, wherein each R′ is independently as described herein. In some embodiments, R′ is R as described herein. In some embodiments, R′ is —H. In some embodiments, each R′ is —H. In some embodiments, R′ is —OR, wherein R is as described herein. In some embodiments, R′ is —OH. In some embodiments, R′ is —N(R)₂ wherein each R is independently as described herein. In some embodiments, R′ is —SR wherein R is as described herein. In some embodiments, R′ is R wherein R is optionally substituted aliphatic, e.g., C₁₋₁₀ aliphatic. In some embodiments, R′ is R wherein R is optionally substituted aliphatic, e.g., C₁₋₁₀ alkenyl. In some embodiments, R′ is R wherein R is optionally substituted aliphatic, e.g., C₁₋₁₀ alkynyl. In some embodiments, —CHR′—CHR′— is —CH₂—CH₂—.

TABLE A-I
Exemplary amino acids (Fmoc-Protected).

| | |
|---|---|
| 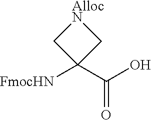 | Monomer A (M_{A}) |
| 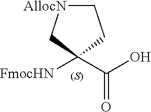 | Monomer B (M_{B}) |
| 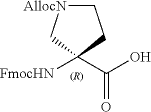 | Monomer C (M_{C}) |

TABLE A-II
Exemplary amino acids (Fmoc-Protected).

| | |
|---|---|
| 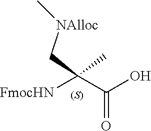 | Monomer D (M_{D}) |
| 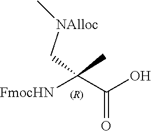 | Monomer E (M_{E}) |
| 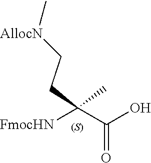 | Monomer F (M_{F}) |
| 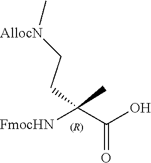 | Monomer G (M_{G}) |
| 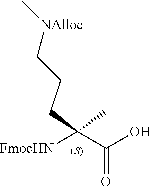 | Monomer H (M_{H}) |
| 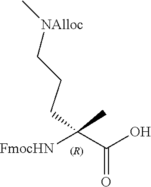 | Monomer I (M_{I}) |

TABLE A-III
Exemplary amino acids (Fmoc-Protected).

| | |
|---|---|
| 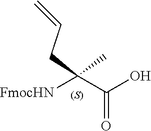 | S3 |
| 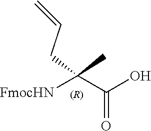 | R3 |
| 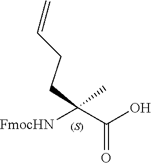 | S4 |
| 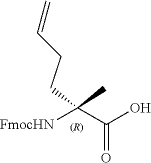 | R4 |
| 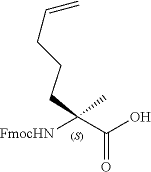 | S5 |
| 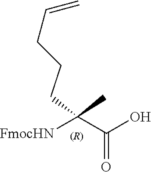 | R5 |
| 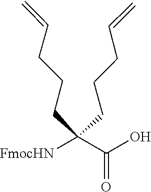 | B5 |
| 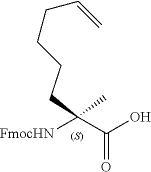 | S6 |
| 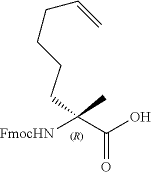 | R6 |
| 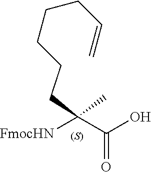 | S7 |
| 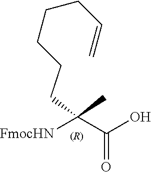 | R7 |
| 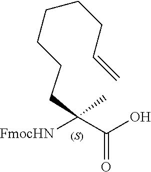 | S8 |
| 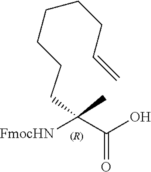 | R8 |
| 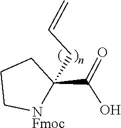 | |
| n is 1-5 e.g., 1, 2 or 3 | |
| 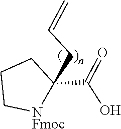 | |
| n is 1-5, e.g., 1, 2 or 3 | |
| 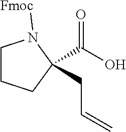 | PL3 |
| 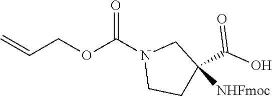 | PyrS |
| 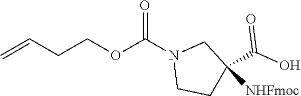 | PyrS1 |
| 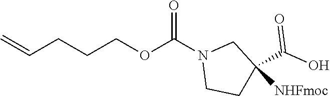 | PyrS2 |
| 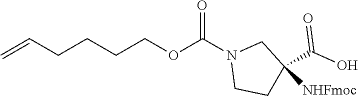 | PyrS3 |
| 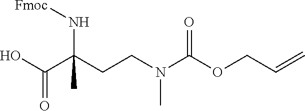 | RdN |
| 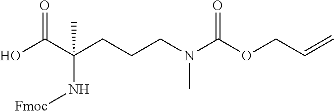 | ReN |
| 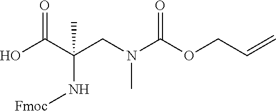 | RgN |
| 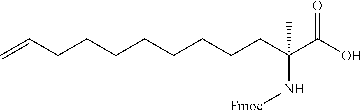 | S₁₀ |
| 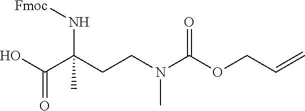 | SdN |
| 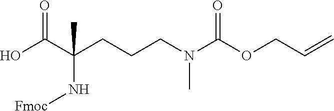 | SeN |
| 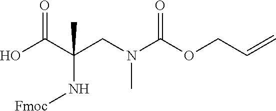 | SgN |

In some embodiments, an amino acid is an alpha-amino acid. In some embodiments, an amino acid is an L-amino acid. In some embodiments, an amino acid is a D-amino acid. In some embodiments, the alpha-carbon of an amino acid is achiral. In some embodiments, an amino acid is a beta-amino acid. In some embodiments, an amino acid is a gamma-amino acid.

In some embodiments, a provided amino acid sequence contains two or more amino acid residues whose side chains are linked together to form one or more staples. In some embodiments, a provided amino acid sequence contains two or more amino acid residues, each of which independently has a side chain comprising an olefin. In some embodiments, a provided amino acid sequence contains two or more amino acid residues, each of which independently has a side chain comprising a terminal olefin. In some embodiments, a provided amino acid sequence contains two and no more than two amino acid residues, each of which independently has a side chain comprising an olefin. In some embodiments, a provided amino acid sequence contains two and no more than two amino acid residues, each of which independently has a side chain comprising a terminal olefin. In some embodiments, a provided amino acid sequence comprises at least one residue of an amino acid that comprises an olefin and a nitrogen atom other than the nitrogen atom of its amino group. In some embodiments, a provided amino acid sequence comprises at least one residue of an amino acid that comprises a terminal olefin and a nitrogen atom other than the nitrogen atom of its amino group. In some embodiments, a provided amino acid sequence comprises at least one residue of an amino acid that has a side chain than comprises a terminal olefin and a nitrogen atom. In some embodiments, a provided amino acid sequence comprises at least one residue of an amino acid of formula A-I, wherein R² comprising an olefin and a —N(R′)— moiety, wherein R′ is as described in the present disclosure (including, in some embodiments, optionally taken together with R³ and their intervening atoms to form an optionally substituted ring as described in the present disclosure). In some embodiments, R² comprising a terminal olefin and a —N(R′)— moiety wherein R′ is as described in the present disclosure. In some embodiments, a provided amino acid sequence comprises at least one residue of an amino acid selected from Table A-I. In some embodiments, a provided amino acid sequence comprises at least one residue of an amino acid selected from Table A-II. In some embodiments, a provided amino acid sequence comprises at least one residue of an amino acid selected from Table A-III. In some embodiments, two olefins from two side chains are linked together through olefin metathesis to form a staple. In some embodiments, a staple is preferably formed by side chains of amino acid residues that are not at the corresponding positions of a target of interest. In some embodiments, a formed staple does not disrupt interaction between the peptide and a target of interest.

In some embodiments, a provided staple is a hydrocarbon staple. In some embodiments, a hydrocarbon staple comprises no chain heteroatoms wherein a chain of a staple is the shortest covalent connection within the staple from one end of the staple to the other end of the staple.

In some embodiments, an olefin in a staple is a Z-olefin. In some embodiments, an olefin in a staple in an E-olefin. In some embodiments, a provided composition comprises stapled peptides comprising a staple that contains a Z-olefin and stapled peptides comprising a staple that contains an E-olefin. In some embodiments, a provided composition comprises stapled peptides comprising a staple that contains a Z-olefin. In some embodiments, a provided composition comprises stapled peptides comprising a staple that contains an E-olefin. In some embodiments, otherwise identical stapled peptides that differ only in the E Z configuration of staple olefin demonstrate different properties and/or activities as demonstrated herein. In some embodiments, stapled peptides with E-olefin in a staple may provide certain desirable properties and/or activities given the context. In some embodiments, stapled peptides with Z-olefin in a staple may provide certain desirable properties and/or activities given the context.

In some embodiments, the present disclosure provides compositions comprising stapled peptides. In some embodiments, a composition comprises one and only one stereoisomer of a stapled peptide (e.g., E or Z isomer, and/or a single diastereomer/enantiomer with respect to a chiral center, etc.). In some embodiments, a composition comprises two or more stereoisomers (e.g., both E and Z isomers of one or more double bonds, and/or one or more diastereomers/enantiomers with respect to a chiral center, etc.). In some embodiments, a composition corresponds to a single peak in a chromatographic separation, e.g., HPLC. In some embodiments, a peak comprises one and only one stereoisomers. In some embodiments, a peak comprises two or more stereoisomers.

In some embodiments, two staples may be bonded to the same atom of the peptide backbone, forming a stitched peptide.

In some embodiments, a staple is pro-lock wherein one end of the staple is bonded to the alpha-carbon of a proline residue.

Certain useful staples are described in, e.g., WO 2019/051327, WO 2022/020652, WO/2022/261257, etc., the staples of each of which are incorporated herein by reference.

In some embodiments, the double bond in a (i, i+3) staple is Z. In some embodiments, the double bond in a (i, i+4) staple is Z. In some embodiments, the double bond in a (i, i+7) staple is Z. In some embodiments, the double bond in a (i, i+3) staple is E. In some embodiments, the double bond in a (i, i+4) staple is E. In some embodiments, the double bond in a (i, i+7) staple is E.

In some embodiments, a staple comprises —S—. In some embodiments, stapling technologies comprise utilization of one or more, e.g., two or more, sulfur-containing moieties. In some embodiments, a stapled peptide comprises cysteine stapling. In some embodiments, two cysteine residues are stapled wherein the —S— moieties of the two cysteine residues are connected optionally through a linker. In some embodiments, a stapled peptide comprises one and no more than one staples from cysteine stapling.

In some embodiments, the present disclosure provides useful technologies relating to cysteine stapling. Among other things, the present disclosure appreciates that peptides amenable to cysteine stapling and/or comprising one or more cysteine staples, can be produced and/or assessed in a biological system. The present disclosure further appreciates that certain such systems permit development, production, and/or assessment of cysteine stapled peptides having a range of different structures (e.g., different amino acid sequences), and in fact can provide a user with complete control over selection and implementation of amino acid sequences to be incorporated into stapled peptides.

Cysteine stapling, as described herein, involves linking one cysteine residue to another cysteine residue, where the resulting bond is not through the peptide backbone between the linked cysteine residues.

In some embodiments, a stapled peptide as described herein comprises a staple which staple is L^{s}, wherein:

L^{s} is -L^{s1}-S-L^{s2}-S-L^{s3}-.
L^{s1} and L^{s3} are each independently L;
L^{s2} is L; and
each L is independently a covalent bond, or an optionally substituted, bivalent C₁-C₂₅ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond; or
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, L^{s2} is L and comprises at least one —C(O)—.

In some embodiments, L is independently a bivalent C₁-C₂₅ aliphatic group. In some embodiments, L is independently a bivalent C₁-C₂₀ aliphatic group. In some embodiments, L is independently a bivalent C₁-C₁₀ aliphatic group. In some embodiments, L is independently a bivalent C₁-C₅ aliphatic group. In some embodiments, L is independently a bivalent C₁ aliphatic group. In some embodiments, L is —CH₂.

In some embodiments, L^{s1} is —CH₂—. In some embodiments, L^{s3} is —CH₂—. In some embodiments, L^{s1} and L^{s3} are both —CH₂—. In some embodiments, L^{s} is —CH₂—S-L^{s2}-S—CH₂—.

In some embodiments, L^{s2} comprises —C(R′)₂-L′—C(R′)₂—, wherein L′ is described in the present disclosure. In some embodiments, L^{s2} is -L^{x1}-C(O)Q-L′-QC(O)-L^{x1}-, wherein each variable is independently as described in the present disclosure. In some embodiments, L^{s2} is —CH₂C(O)Q-L′-QC(O)CH₂—, wherein each —CH₂— is independently and optionally substituted. In some embodiments, L^{s2} is —CH₂C(O)Q-L′-QC(O)CH₂—.

In some embodiments, L^{s2} In some embodiments, L^{s2} is L and comprises at least one —C(O)—. In some embodiments, L^{s2} is L and comprises at least two —C(O)—. In some embodiments, L^{s2} is L and comprises at least one —C(O)Q-, wherein Q is selected from the group consisting of: a covalent bond, —N(R′)—, —O—, and —S—. In some embodiments, L^{s2} is L and comprises at least one —C(O)Q-, wherein Q is selected between —N(R′)— and —O—. In some embodiments, L^{s2} is L and comprises at least two —C(O)Q-, wherein Q is selected from the group consisting of: —N(R′)—, —O—, and —S—. In some embodiments, L^{s2} is L and comprises at least two —C(O)Q-, wherein Q is selected between —N(R′)— and —O—. In some embodiments, L^{s2} is L and comprises at least one —C(O)N(R′)—. In some embodiments, L^{s2} is L and comprises at least two —C(O)N(R′)—. In some embodiments, L^{s2} is L and comprises at least one —C(O)O—. In some embodiments, L^{s2} is L and comprises at least two —C(O)O—.

In some embodiments, L^{s2} comprises -Q-L′-Q-, wherein Q is independently selected from the group consisting of: —N(R′)—, —O—, and —S, wherein L′ is described in the present disclosure.

In some embodiments, L^{s2} comprises -Q-L′-Q-, wherein Q is independently selected between —N(R′)— and —O—, wherein L′ is described in the present disclosure. In some embodiments, L^{s2} comprises —C(O)Q-L′-QC(O)—, wherein Q is independently selected from the group consisting of: —N(R′)—, —O—, and —S, wherein L′ is described in the present disclosure. In some embodiments, L^{s2} comprises —C(O)Q-L′-QC(O)—, wherein Q is independently selected between —N(R′)— and —O, wherein L′ is described in the present disclosure. In some embodiments, L^{s2} comprises —C(R′)₂C(O)Q-L′-QC(O)C(R′)₂—, wherein Q is independently selected from the group consisting of: —N(R′)—, —O—, and —S, wherein L′ is described in the present disclosure. In some embodiments, L^{s2} comprises —C(R′)₂C(O)Q-L′-QC(O)C(R′)₂—, wherein Q is independently selected between —N(R′)— and —O, wherein L′ is described in the present disclosure.

In some embodiments, L^{s2} comprises —N(R′)-L′-N(R′)—, wherein L′ is described in the present disclosure. In some embodiments, L^{s2} comprises —C(O)N(R′)-L′-N(R′)C(O)—, wherein L′ is described in the present disclosure. In some embodiments, L^{s2} is —C(R′)₂C(O)N(R′)-L′-N(R′)C(O)C(R′)₂—, wherein L′ is described in the present disclosure.

In some embodiments, L^{s2} comprises —O(R′)-L′-O(R′)—, wherein L′ is described in the present disclosure. In some embodiments, L^{s2} comprises —C(O)O-L′-OC(O)—, wherein L′ is described in the present disclosure. In some embodiments, L^{s2} is —C(R′)₂C(O)O-L′-OC(O)C(R′)₂—, wherein L′ is described in the present disclosure.

In some embodiments, R′ is an optionally substituted C₁₋₃₀ aliphatic. In some embodiments, R′ is an optionally substituted C₁₋₁₅ aliphatic. In some embodiments, R′ is an optionally substituted C₁₋₁₀ aliphatic. In some embodiments, R′ is an optionally substituted C₁₋₅ aliphatic. In some embodiments, R′ is hydrogen.

In some embodiments, L′ is optionally substituted bivalent C₁-C₁₉ aliphatic. In some embodiments, L′ is optionally substituted bivalent C₁-C₁₅ aliphatic. In some embodiments, L′ is optionally substituted bivalent C₁-C₁₀ aliphatic. In some embodiments, L′ is optionally substituted bivalent C₁-C₉ aliphatic. In some embodiments, L′ is optionally substituted bivalent C₁-C₅ aliphatic. In some embodiments, L′ is optionally substituted bivalent C₁-C₇ aliphatic. In some embodiments, L′ is optionally substituted bivalent C₁-C₆ aliphatic. In some embodiments, L′ is optionally substituted bivalent C₁-C₅ aliphatic. In some embodiments, L′ is optionally substituted bivalent C₁-C₃ aliphatic. In some embodiments, L′ is optionally substituted bivalent C₁-C₂ aliphatic. In some embodiments, L′ is optionally substituted bivalent C₁ aliphatic. In some embodiments, L′ is —CH₂—. In some embodiments, L′ is —(CH₂)₂—. In some embodiments, L′ is —(CH₂)₃—. In some embodiments, L′ is —(CH₂)₄—. In some embodiments, L′ is —(CH₂)₅—. In some embodiments, L′ is —(CH₂)₆—. In some embodiments, L′ is —(CH₂)₇—. In some embodiments, L′ is —(CH₂)₈—.

In some embodiments, L′ is optionally substituted bivalent C₆₋₂₀ aryl ring. In some embodiments, L′ is optionally substituted bivalent C₆₋₁₄ aryl ring. In some embodiments, L′ is optionally substituted bivalent C₆₋₁₀ aryl ring. In some embodiments, L′ is optionally substituted bivalent C₆ aryl ring. In some embodiments, L′ is bivalent C₆ aryl substituted with at least one halogen. In some embodiments, L′ is bivalent C₆ aryl substituted with at least two halogen. In some embodiments, L′ is bivalent C₆ aryl substituted with at least three halogen. In some embodiments, L′ is bivalent C₆ aryl substituted with four halogen. In some embodiments, L′ is bivalent C₆ aryl substituted with at least one fluorine. In some embodiments, L′ is bivalent C₆ aryl substituted with at least two fluorine. In some embodiments, L′ is bivalent C₆ aryl substituted with at least three fluorine. In some embodiments, L′ is bivalent C₆ aryl substituted with four fluorine. In some embodiments, L′ is bivalent C₆ aryl substituted with at least one chlorine. In some embodiments, L′ is bivalent C₆ aryl substituted with at least two chlorine. In some embodiments, L′ is bivalent C₆ aryl substituted with at least three chlorine. In some embodiments, L′ is bivalent C₆ aryl substituted with four chlorine. In some embodiments, L′ is bivalent C₆ aryl substituted at with least one —O(CH₂)₀₋₄CH₃. In some embodiments, L′ is bivalent C₆ aryl substituted with at least two —O(CH₂)₀₋₄CH₃. In some embodiments, L′ is bivalent C₆ aryl substituted with at least three —O(CH₂)₀₋₄CH₃. In some embodiments, L′ is bivalent C₆ aryl substituted with four —O(CH₂)₀₋₄CH₃.

In some embodiments, L′ is bivalent 5-20 membered heteroaryl ring having 1-10 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, sulfur, phosphorus and silicon. In some embodiments, L′ is bivalent 5-6 membered heteroaryl ring having 1-4 heteroatoms independently selected from oxygen, nitrogen, and sulfur. In some embodiments, L′ is bivalent 6 membered heteroaryl ring having 1-2 heteroatoms independently selected from oxygen, nitrogen, and sulfur. In some embodiments, L′ is bivalent 6 membered heteroaryl ring having 2 nitrogen.

In some embodiments, L′ is optionally substituted bivalent C₃₋₂₀ cycloaliphatic ring. In some embodiments, L′ is optionally substituted bivalent C₃₋₁₅ cycloaliphatic ring. In some embodiments, L′ is optionally substituted bivalent C₃₋₁₀ cycloaliphatic ring. In some embodiments, L′ is optionally substituted bivalent C₃₋₉ cycloaliphatic ring. In some embodiments, L′ is optionally substituted bivalent C₃₋₈ cycloaliphatic ring. In some embodiments, L′ is optionally substituted bivalent C₃₋₇ cycloaliphatic ring. In some embodiments, L′ is optionally substituted bivalent C₃₋₆ cycloaliphatic ring. In some embodiments, L′ is optionally substituted bivalent C₃₋₅ cycloaliphatic ring. In some embodiments, L′ is optionally substituted bivalent C₃₋₄ cycloaliphatic ring. In some embodiments, L′ is optionally substituted bivalent C₃ cycloaliphatic ring. In some embodiments, L′ is optionally substituted bivalent C₄ cycloaliphatic ring. In some embodiments, L′ is optionally substituted bivalent C₅ cycloaliphatic ring. In some embodiments, L′ is optionally substituted bivalent C₅ cycloalkyl ring. In some embodiments, L′ is optionally substituted bivalent C₅ cycloalkenyl ring. In some embodiments, L′ is optionally substituted bivalent C₆ cycloaliphatic ring. In some embodiments, L′ is optionally substituted bivalent C₆ cycloalkyl ring.

In some embodiments, L^{s2} comprises —N(R′)-L′-N(R′)— and L′ is a covalent bond. In some embodiments L^{s2} comprises —N(R)—N(R)—, wherein:
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments L^{s2} comprises —N(R)—N(R)—, wherein:
each R is independently optionally substituted C₁₋₃₀ aliphatic; or
two or more R groups on two or more atoms are optionally and independently taken together with their intervening atoms to form an optionally substituted, 3-30 membered monocyclic ring.

In some embodiments, L^{s2} is a staple selected from the group consisting of:

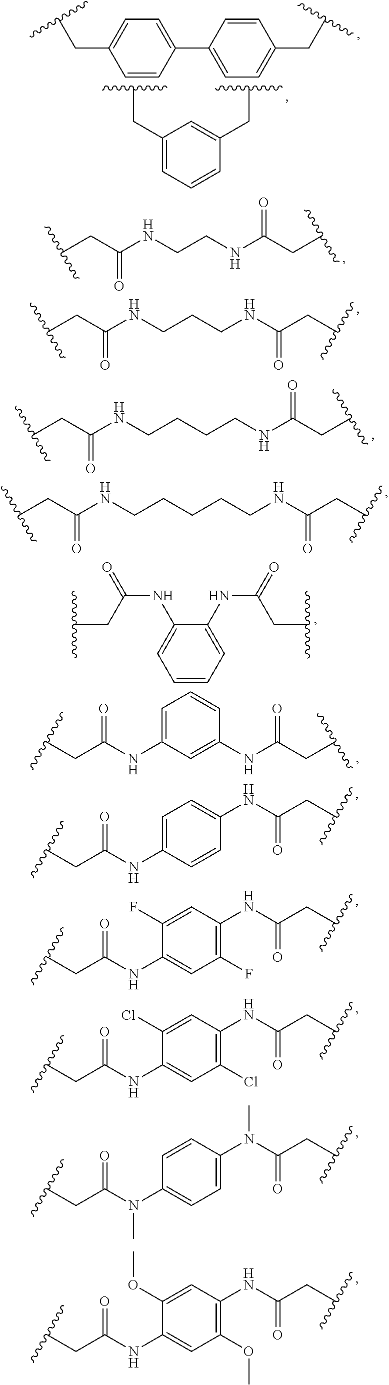

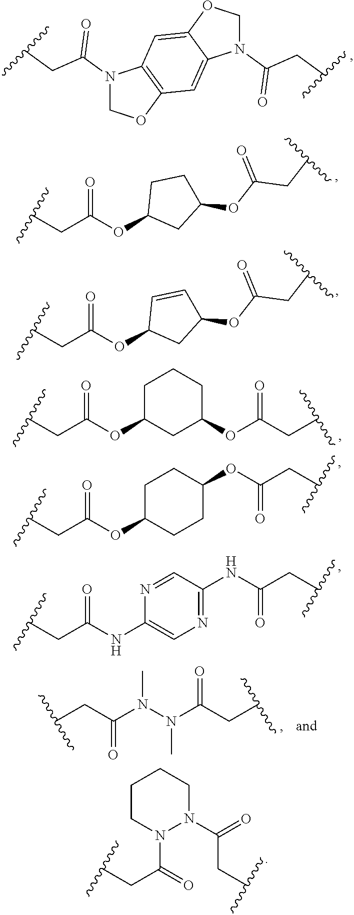

In some embodiments, L^{s1} is optionally substituted bivalent C₁₋₆ aliphatic. In some embodiments, L^{s1} is bivalent C₁₋₆ aliphatic. In some embodiments, L^{s1} is bivalent C₁₋₄ aliphatic. In some embodiments, L^{s1} is saturated. In some embodiments, L^{s1} is linear. In some embodiments, L^{s1} is branched. In some embodiments, L^{s1} is optionally substituted —CH₂—. In some embodiments, L^{s1} is —CH₂—. In some embodiments, L^{s1} is optionally substituted —CH₂—CH₂—. In some embodiments, L^{s1} is —CH₂—CH₂—. In some embodiments, L^{s1} is optionally substituted —C(CH₃)₂—. In some embodiments, L^{s1} is —C(CH₃)₂—.

In some embodiments, L^{s2} is optionally substituted bivalent C₁₋₆, (e.g., C₃₋₆, C₃, C₄, C₅, C₆, etc.) aliphatic wherein one or more methylene units are optionally and independently replaced with -Cy- or —C(R′)₂—. In some embodiments, L^{s2} is optionally substituted bivalent C₁₋₆ aliphatic. In some embodiments, L^{s2} is optionally substituted bivalent C₃₋₆ aliphatic. In some embodiments, L^{s2} is bivalent C₁₋₆ aliphatic. In some embodiments, L^{s2} is bivalent C₁₋₄ aliphatic. In some embodiments, L^{s2} is optionally substituted bivalent C₂ aliphatic. In some embodiments, L^{s2} is optionally substituted bivalent C₃ aliphatic. In some embodiments, L^{s2} is optionally substituted bivalent C₄ aliphatic. In some embodiments, L^{s2} is optionally substituted bivalent C₅ aliphatic. In some embodiments, L^{s2} is optionally substituted bivalent C₆ aliphatic. In some embodiments, L^{s2} is substituted. In some embodiments, L^{s2} is unsubstituted. In some embodiments, L^{s2} is saturated. In some embodiments, L^{s2} is linear. In some embodiments, L^{s2} is branched. In some embodiments, L^{s2} is optionally substituted bivalent C₃₋₆, (e.g., C₃₋₅, C₃, C₄, C₅, C₆, etc.) aliphatic wherein one or two methylene units are independently replaced with -Cy-. In some embodiments, L^{s2} is —CH₂—Cy-CH₂—. In some embodiments, L^{s2} is —CH₂—CH₂—Cy-CH₂—CH₂—. In some embodiments, L^{s2} is —CH₂—Cy-Cy-CH₂—. Various useful embodiments of -Cy- are as described herein. For example, in some embodiments, -Cy- is an optionally substituted monocyclic 5-membered aromatic ring having 0-4 heteroatoms. In some embodiments, -Cy- is an optionally substituted monocyclic 6-membered aromatic ring having 0-4 heteroatoms. In some embodiments, -Cy- is optionally substituted phenylene. In some embodiments, -Cy- is optionally substituted 1,2-phenylene. In some embodiments, -Cy- is 1,2-phenylene. In some embodiments, -Cy- is optionally substituted 1,3-phenylene. In some embodiments, -Cy- is 1,3-phenylene. In some embodiments, -Cy- is optionally substituted 1,5-phenylene. In some embodiments, -Cy- is 1,5-phenylene. In some embodiments, -Cy- is 3-methyl-1,5-phenylene. In some embodiments, -Cy- is 3-methoxy-1,5-phenylene. In some embodiments, -Cy- is an optionally substituted bivalent pyridyl ring. In some embodiments, -Cy- is optionally substituted

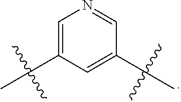 [SMILES: *c1cncc(*)c1]

In some embodiments, -Cy- is

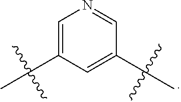 [SMILES: *c1cncc(*)c1]

In some embodiments, -Cy- is optionally substituted

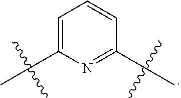 [SMILES: *c1cccc(*)n1]

In some embodiments, -Cy- is

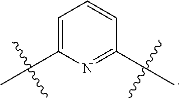 [SMILES: *c1cccc(*)n1]

In some embodiments, -Cy- is an optionally substituted bicyclic 9-membered aromatic ring having 0-4 heteroatoms. In some embodiments, -Cy- is an optionally substituted bicyclic 10-membered aromatic ring having 0-4 heteroatoms. In some embodiments, -Cy- is an optionally substituted bivalent naphthyl ring. In some embodiments, -Cy- is a bivalent naphthyl ring. In some embodiments, -Cy- is optionally substituted

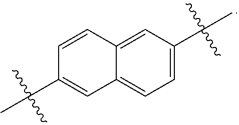 [SMILES: *c1ccc2cc(*)ccc2c1]

In some embodiments, -Cy- is

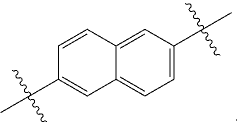 [SMILES: *c1ccc2cc(*)ccc2c1]

In some embodiments, -Cy- is optionally substituted

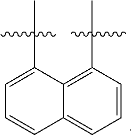 [SMILES: *c1cccc2cccc(*)c12]

In some embodiments, -Cy- is

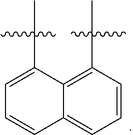 [SMILES: *c1cccc2cccc(*)c12]

In some embodiments, -Cy- is an optionally substituted 3-10 (e.g., 5-10, 5-6, 3, 4, 5, 6, 7, 8, 9, 10, etc.) membered bivalent cycloaliphatic ring. In some embodiments, it is saturated. In some embodiments, -Cy- is an optionally substituted 6-membered cycloalkyl ring. In some embodiments, -Cy- is optionally substituted

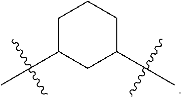 [SMILES: *C1CCCC(*)C1]

In some embodiments, -Cy- is

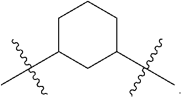 [SMILES: *C1CCCC(*)C1]

In some embodiments, L^{s2} is optionally substituted bivalent C₃₋₆, (e.g., C₃₋₅, C₃, C₄, C₅, C₆, etc.) aliphatic wherein one or two methylene units are independently replaced with —C(R′)₂—. In some embodiments, L^{s2} is —CH₂—C(R′)₂—CH₂—. In some embodiments, the two R′ are taken together with the carbon atom to form an optionally substituted ring as described herein, e.g., an optionally substituted 3-10 (e.g., 5-10, 5-6, 3, 4, 5, 6, 7, 8, 9, 10, etc.) membered ring having 0-4 (e.g., 1-4, 0, 1, 2, 3, 4, etc.) heteroatoms. In some embodiments, a ring is saturated. In some embodiments, a ring has one or more heteroatoms. In some embodiments, —C(R′)₂— is

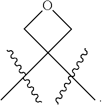 [SMILES: *C1(*)COC1]

In some embodiments, L^{s2} is optionally substituted

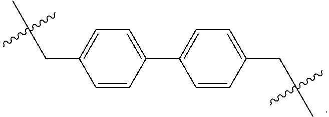 [SMILES: *Cc1ccc(-c2ccc(C*)cc2)cc1]

In some embodiments, L^{s2} is optionally substituted

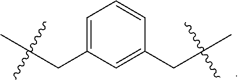 [SMILES: *Cc1cccc(C*)c1]

In some embodiments, L^{s2} is optionally substituted

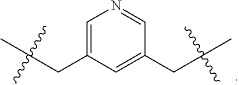 [SMILES: *Cc1cncc(C*)c1]

In some embodiments, L^{s2} is optionally substituted

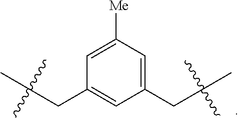 [SMILES: *Cc1cc(C)cc(C*)c1]

In some embodiments, L^{s2} is optionally substituted

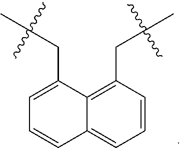 [SMILES: *Cc1cccc2cccc(C*)c12]

In some embodiments, L^{s2} is optionally substituted

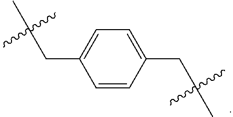 [SMILES: *Cc1ccc(C*)cc1]

In some embodiments, L^{s2} is optionally substituted

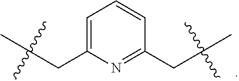 [SMILES: *Cc1cccc(C*)n1]

### In

some embodiments, L^{s2} is optionally substituted

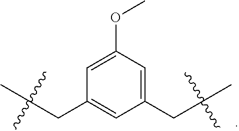 [SMILES: *Cc1cc(C*)cc(OC)c1]

In some embodiments, L^{s2} is optionally substituted

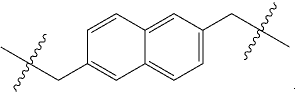 [SMILES: *Cc1ccc2cc(C*)ccc2c1]

In some embodiments, L^{s2} is optionally substituted

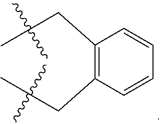 [SMILES: *Cc1ccccc1C*]

In some embodiments, L^{s2} is optionally substituted —(CH₂)₄—. In some embodiments, L^{s2} is optionally substituted —(CH₂)₃—. In some embodiments, L^{s2} is optionally substituted —CH₂—CH═CH—CH₂—. In some embodiments, L^{s2} is optionally substituted (E)-CH₂—CH═CH—CH₂—. In some embodiments, L^{s2} is optionally substituted —CH₂—C(O)—CH₂—. In some embodiments, L^{s2} is optionally substituted

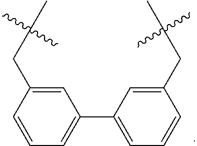 [SMILES: *Cc1cccc(-c2cccc(C*)c2)c1]

In some embodiments, L^{s2} is optionally substituted

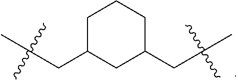 [SMILES: *CC1CCCC(C*)C1]

In some embodiments, L^{s2} is optionally substituted

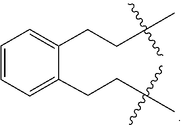 [SMILES: *CCc1ccccc1CC*]

In some embodiments, L^{s2} is optionally substituted

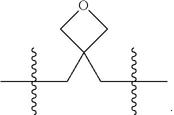 [SMILES: *CC1(C*)COC1]

In some embodiments, it is substituted. In some embodiments, it is unsubstituted. In some embodiments,

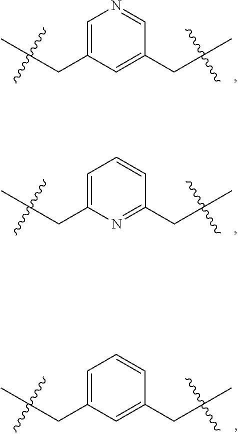 [SMILES: *Cc1cncc(C*)c1 ; *Cc1cccc(C*)n1 ; *Cc1cccc(C*)c1]

—(CH₂)₄—, (E)-CH₂—CH═CH—CH₂—, —(CH₂)₃—, and/or —CH₂—C(O)—CH₂— provide higher binding and/or potency than

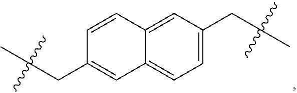 [SMILES: *Cc1ccc2cc(C*)ccc2c1]

and/or

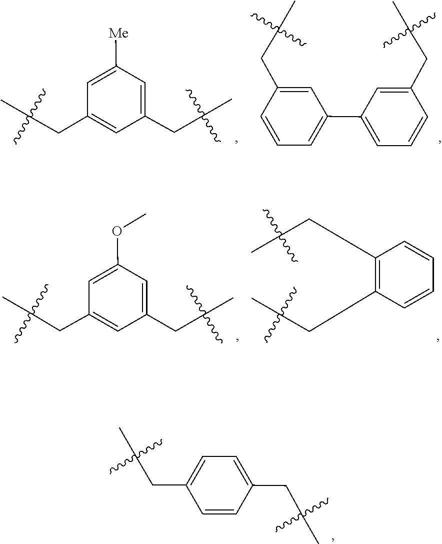 [SMILES: *Cc1cc(C)cc(C*)c1 ; *Cc1cccc(-c2cccc(C*)c2)c1 ; *Cc1cc(C*)cc(OC)c1 ; *Cc1ccccc1C* ; *Cc1ccc(C*)cc1]

under comparable conditions.

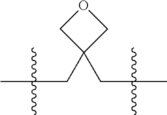 [SMILES: *CC1(C*)COC1]

In some embodiments, L^{s3} is optionally substituted bivalent C₁₋₆ aliphatic. In some embodiments, L^{s3} is bivalent C₁₋₆ aliphatic. In some embodiments, L^{s3} is bivalent C₁₋₄ aliphatic. In some embodiments, L^{s3} is saturated. In some embodiments, L^{s3} is linear. In some embodiments, L^{s3} is branched. In some embodiments, L^{s3} is optionally substituted —CH₂—. In some embodiments, L^{s3} is —CH₂—. In some embodiments, L^{s3} is optionally substituted —CH₂—CH₂—. In some embodiments, L^{s3} is —CH₂—CH₂—. In some embodiments, L^{s3} is optionally substituted —C(CH₃)₂—. In some embodiments, L^{s3} is —C(CH₃)₂—.

In some embodiments, an amino acid residue for forming a staple is selected from:

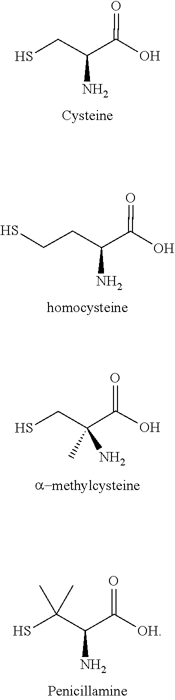

In some embodiments, both amino acid residue for forming a staple are independently residues of these amino acids. In some embodiments, each of L^{s1} and L^{s3} is independently —CH₂—, —CH₂—CH₂—, or —C(CH₃)₂—. In some embodiments, a staple is formed by reacting the thiol groups with a thiol reactive linker compound. In some embodiments, such a linker compound has the structure of LG-L^{s2}-LG or a salt thereof, wherein each LG is independently a leaving group, e.g., —Br, —I, etc. In some embodiments, each LG is independently —Br or —I. In some embodiments, each LG is —Br. In some embodiments, each LG is —I. In some embodiments, L^{s2} are of such structures that LG-L^{s2}-LG (each LG is independently —Br or —I) is a compound selected from:

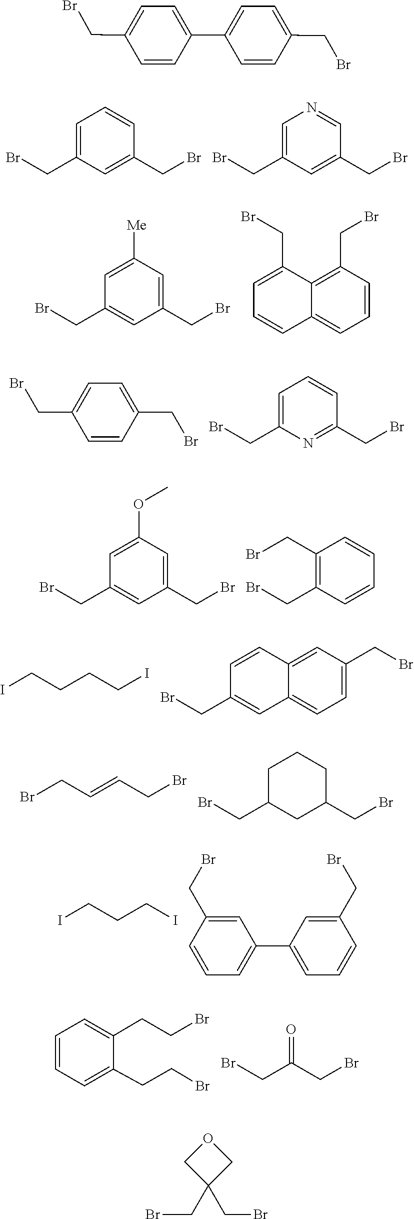

Various technologies are available for constructing of thioether staples. For example, in some embodiments, a peptide and excess equivalents (e.g., about 2-10, 5-10, 2, 3, 4, 5, 6, 7, 8, 9, 10, etc.; in some embodiments, 5) of a linker compound were added to a 1:1 DMF: 100 mM Na₂CO₃ pH 8.0 solution and stirred at a suitable temperature, e.g., room temperature for a suitable period of time, in some embodiments, 1-2 hours. In some embodiments, e.g., for relatively weaker electrophiles, excess equivalents (e.g., about 10-30, 10-20, 10, 20, etc.; in some embodiments, 20) of a metal salt, e.g., Zn(acac)₂ and an excess equivalents (e.g., about 5-20, 10-15, 10, 15, 20, etc.; in some embodiments, 10-15) of a linker compound were added to a peptide in DMA, and the mixture was stirred for a suitable period of time, e.g., overnight, at a suitable temperature, e.g., 37° C. In some embodiments, equivalents of Zn(acac)₂ and linker compounds were doubled, and/or the temperature was increased to 50° C. In some embodiments, certain linker compounds react better than others. For example, in some embodiments,

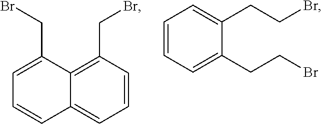

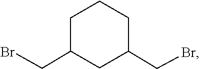

or

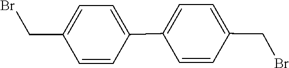 [SMILES: BrCc1ccc(-c2ccc(CBr)cc2)cc1]

provides poor reaction yields or failed reactions. Those skilled in the art appreciate that other technologies may be utilized to introduce the corresponding linker moieties (L^{s2}), e.g., through utilizing other leaving groups or through other reaction mechanisms/routes.

In some embodiments, a staple having the structure of -L^{s1}-S-L^{s2}-S-L^{s3}- is a (i, i+4) staple. In some embodiments, such a staple is in closer to a C-terminus. In some embodiments, such a staple is in closer to a N-terminus. For example, in some embodiments, such a staple is between X¹⁰ and X¹⁴.

In some embodiments, certain staples provide better properties and/or activities. For example, in some embodiments, based on target binding affinity certain staples/scaffolds is ranked in the following order:

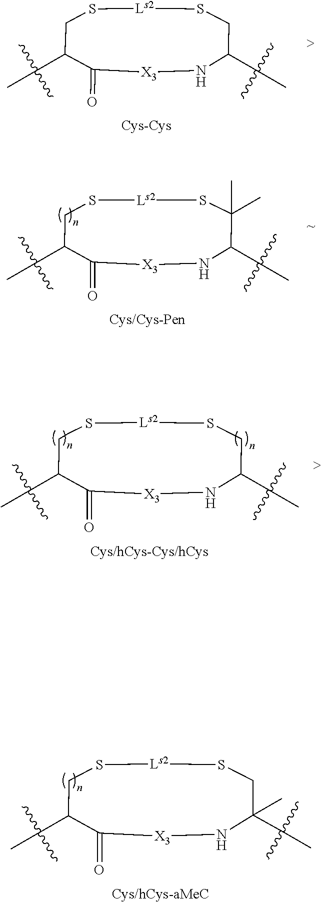

As those skilled in the art will appreciate, provided technologies can be utilized to prepare peptides using non-cysteine residues and suitable chemistry therefor. For example, in some embodiments, cysteine stapling is replaced with lysine stapling, wherein the cysteine residues for cysteine stapling are replaced with lysine residues for lysine stapling (e.g., using agents that can crosslink two lysine residues, for example, through reactions with side chain amino groups). In some embodiments, for lysine stapling, R^{E} in various formulae is or comprises an activated carboxylic acid group (e.g., NHS ester group), an imidoester group, etc. Suitable reagents are widely known in the art including many commercially available ones. In some embodiments, cysteine stapling is replaced with methionine stapling. In some embodiments, cysteine residues for cysteine stapling are replaced with methionine residues for methionine stapling. In some embodiments, cysteine stapling is replaced with tryptophan stapling. In some embodiments, cysteine residues for cysteine stapling are replaced with tryptophan residues for tryptophan stapling. In some embodiments, cysteine residues for cysteine stapling are replaced with amino acid residues comprising olefins which can form staples, e.g., via metathesis. In some embodiments, cysteine residues for cysteine stapling are replaced with amino acid residues comprising —C(O)OH and amino groups or salt forms thereof which can form staples, e.g., through amidation. In some embodiments, a cysteine staple between by two cysteine residues is replaced with a non-cysteine staple as described herein. As those skilled in the art will appreciate, various technologies (e.g., reagents, reactions, etc.) are described in the art and can be utilized in accordance with the present disclosure for, e.g., methionine stapling, tryptophan stapling, etc. In some embodiments, such stapling can be performed using reagents having various formulae described herein, wherein R^{E} is or comprises a group that are suitable for methionine and/or tryptophan stapling. In some embodiments, stapling may be performed using one residue at a first position, and a different residue at a second position. Useful reagents for such stapling may comprise a first reactive group for stapling at a first position (e.g., through a first R^{E}), and a second reactive group for stapling at a second position (e.g., through a second R^{E}).

In some embodiments, for various types of stapling (e.g., cysteine stapling, or non-cysteine stapling), stapling is between residues (e.g., cysteine residues for cysteine stapling) separated by two residues (i+3 stapling). In some embodiments, stapling is between residues separated by three residues (i+4 stapling). In some embodiments, stapling is between residues separated by six residues (i+7 stapling).

As appreciated by those skilled in the art, in some embodiments, more than two residues can be stapled at the same time. For example, in some embodiments, three or more cysteines are stapled using crosslinking reagents containing three or more reactive groups (e.g., R^{E} groups).

In some embodiments, as described herein, the present disclosure provides useful technologies relating to non-cysteine stapling. Among other things, the present disclosure appreciates that peptides amenable to cysteine stapling and/or comprising one or more non-cysteine staples, can have its cysteine residues and cysteine staple replaced with other amino acids and staples described herein (e.g. hydrocarbon and other non-hydrocarbon amino acid and staples). In some embodiments, the resulting non-cysteine stapled peptide maintains the same or similar interaction with a target of interest when compared to a reference cysteine stapled peptide.

Agents, e.g., peptides including stapled peptides, can contain various numbers of amino acid residues. In some embodiments, a length of a peptide agent is about 5-20, 5-19, 5-18, 5-17, 5-16, 5-15, 10-20, 10-19, 10-18, 10-17, 10-16, 10-15, 11-20, 11-19, 11-18, 11-17, 11-16, 11-15, 12-20, 12-19, 12-18, 12-17, 12-16, 12-15, 13-20, 13-19, 13-18, 13-17, 13-16, 13-15, 14-20, 14-19, 14-18, 14-17, 14-16, 14-15, or about 2, 3, 4, 5, 6, 7, 8, 9, 10, 11, 12, 13, 14, 15, 16, 17, 18, 19, or 20 amino acid residues. In some embodiments, a length is about 10 amino acid residues. In some embodiments, a length is about 11 amino acid residues. In some embodiments, a length is about 12 amino acid residues. In some embodiments, a length is about 13 amino acid residues. In some embodiments, a length is about 14 amino acid residues. In some embodiments, a length is about 15 amino acid residues. In some embodiments, a length is about 16 amino acid residues. In some embodiments, a length is about 17 amino acid residues. In some embodiments, a length is about 18 amino acid residues. In some embodiments, a length is about 19 amino acid residues. In some embodiments, a length is about 20 amino acid residues. In some embodiments, a length is about 20 or more amino acid residues.

In some embodiments, as described herein, one or more staples independently comprise an olefin double bond (e.g., formed through olefin metathesis). In some embodiments, one or more staples independently comprise an amide group (e.g., formed through amidation). In some embodiments, at least one staple does not contain an olefin double bond. In some embodiments, there is at least one staple whose formation does not comprise reactions of olefins such as olefin metathesis and/or modification of olefin double bonds (e.g., hydrogenation, epoxidation, etc.).

Certain useful staples are described in the “Agents” section, below.

Certain stapled peptides are described in WO 2022/020652 and PCT/US2022/032738, the stapled peptides of each of which are independently incorporated herein by reference.

### MDM2

E3 ubiquitin-protein ligase Mdm2 is reported to have many reported functions. For example, it is reported to be a negative regulatory factor of p53. According to some reports, MDM2 can function as an E3 ubiquitin ligase that mediates ubiquitination of various polypeptides, e.g., p53. It is reported that MDM2 may inhibit p53- and p73-mediated cell cycle arrest and apoptosis.

MDM2 amplification has been reported in many human malignancies, including lung cancer and colon cancer and, according to some reports, MDM2 overexpression may be associated with chemotherapeutic resistance in human malignancies.

Among other things, the present disclosure provides technologies that can modulate MDM2 functions. In some embodiments, provided agents bind to MDM2, e.g., at its p53 binding domain. In some embodiments, provided agents compete with p53 for MDM2 binding. In some embodiments, the present disclosure provides technologies for preventing or treating MDM2-associated conditions, disorders or diseases.

### CHIP

E3 ubiquitin-protein ligase CHIP (CHIP) is reported to have many functions. For example, it is reported to be an E3 ubiquitin-protein ligase that mediates ubiquitination of various polypeptides. According to some reports, it can target misfolded chaperone substrates towards proteasomal degradation.

There are reports that CHIP is involved in multiple conditions, disorders or diseases, e.g., various forms of spinocerebellar ataxia (e.g., SCAR16, SCA48, etc.).

Among other things, the present disclosure provides technologies that can modulate CHP functions. In some embodiments, provided agents bind to CHIP, e.g., a TPR domain of CHIP. In some embodiments, provided agents compete with a N-terminal sequence of HSP70 (e.g., SSGPTIEEVD (SEQ ID NO: 3)) for CHIP binding.

In some embodiments, the present disclosure provides technologies for preventing or treating CHIP-associated conditions, disorders or diseases.

### Binding Sites

In some embodiments, agents can bind to polypeptides, e.g., MDM2 or CHIP polypeptides. Certain MDM2 and CHIP binding sites are described below. In some embodiments, interacting residues in polypeptides, e.g., MDM2 or CHIP polypeptides, are within a certain distance, e.g., 4 or 4.5 angstroms of agents in crystallographic structures. For example, in some embodiments, certain residues are within 4 angstroms of agents. In some embodiments, certain residues are within 4.5 angstroms of agents.

### MDM2 Binding Sites

In some embodiments, agents, e.g., peptides, interact with MDM2 or a portion thereof. In some embodiments, agents, e.g., peptides, interact with polypeptides comprising characteristic portions and/or residues of MDM2. In some embodiments, agents, e.g., peptides, interact with polypeptides that are or comprise p53 binding domain of MDM2.

For example, in some embodiments, an agent interacts with one or more residues each of which independently is or corresponds to Ala21, Ser22, Glu23, Gln24, Glu25, Thr26, Thr49, Met50, Lys51, Leu54, Phe55, Leu57, Gly58, Gln59, Ile61, Met62, Tyr67, Gln72, His73, Val75, Phe91, Val93, Lys94, His96, Arg97, Ile99, Tyr100, Ile103, and Tyr104 of SEQ ID NO: 1. In some embodiments, an agent interacts with one or more residues each of which independently is or corresponds to Ser22, Thr26, Met50, Lys51, Leu54, Phe55, Leu57, Gly58, Gln59, Ile61, Met62, Tyr67, Gln72, His73, Val75, Phe91, Val93, Lys94, His96, Arg97, Ile99, Tyr100, Ile103, and Tyr104 of SEQ ID NO: 1. In some embodiments, an agent, e.g., an agent that is or comprises cluster C41 or H101, interacts with one or more residues each of which independently is or corresponds to Met50, Lys51, Leu54, Phe55, Leu57, Gly58, Ile61, Met62, Tyr67, Gln72, His73, Val75, Val93, Lys94, His96, Arg97, Ile99, and Tyr100 of SEQ ID NO: 1. In some embodiments, an agent, e.g., an agent that is or comprises cluster C42 or H102, interacts with one or more residues each of which independently is or corresponds to Met50, Lys51, Leu54, Phe55, Leu57, Gly58, Gln59, Ile61, Met62, Tyr67, Gln72, His73, Val75, Phe91, Val93, Lys94, His96, Arg97, Ile99, Tyr100, Ile103, and Tyr104 of SEQ ID NO: 1. In some embodiments, an agent, e.g., an agent that is or comprises cluster C43 or H103, interacts with one or more residues each of which independently is or corresponds to Ser22, Thr26, Met50, Lys51, Leu54, Phe55, Leu57, Gly58, Ile61, Met62, Tyr67, Gln72, His73, Val75, Val93, Lys94, His96, Arg97, Ile99, and Tyr100 of SEQ ID NO: 1.

In some embodiments, an agent interacts with one or more residues each of which independently is or corresponds to Ser22, Gln24, Thr26, Thr49, Met50, Lys51, Leu54, Phe55, Leu57, Gly58, Gln59, Ile61, Met62, Tyr67, Gln72, His73, Val75, Val93, Lys94, His96, Arg97, Ile99, Tyr100, Ile103, and Tyr104 of SEQ ID NO: 1. In some embodiments, an agent interacts with one or more residues each of which independently is or corresponds to Ser22, Met50, Lys51, Leu54, Phe55, Leu57, Gly58, Gln59, Ile61, Met62, Tyr67, Gln72, His73, Val75, Val93, Lys94, His96, Arg97, Ile99, Tyr100, Ile103, and Tyr104 of SEQ ID NO: 1. In some embodiments, an agent, e.g., an agent that is or comprises cluster C41 or H101, interacts with one or more residues each of which independently is or corresponds to Met50, Lys51, Leu54, Phe55, Leu57, Gly58, Ile61, Met62, Tyr67, Gln72, His73, Val93, Lys94, His96, Arg97, Ile99, and Tyr100 of SEQ ID NO: 1. In some embodiments, an agent, e.g., an agent that is or comprises cluster C42 or H102, interacts with one or more residues each of which independently is or corresponds to Met50, Lys51, Leu54, Phe55, Leu57, Gly58, Gln59, Ile61, Met62, Tyr67, Gln72, His73, Val75, Val93, Lys94, His96, Arg97, Ile99, Tyr100, Ile103, and Tyr104 of SEQ ID NO: 1. In some embodiments, an agent, e.g., an agent that is or comprises cluster C43 or H103, interacts with one or more residues each of which independently is or corresponds to Ser22, Met50, Lys51, Leu54, Phe55, Gly58, Ile61, Met62, Tyr67, Gln72, His73, Val75, Val93, His96, Arg97, and Ile99 of SEQ ID NO: 1.

In some embodiments, an agent interacts with a residue that is or corresponds to Ala21 of SEQ ID NO: 1. In some embodiments, an agent interacts with a residue that is or corresponds to Ser22 of SEQ ID NO: 1. In some embodiments, an agent interacts with a residue that is or corresponds to Glu23 of SEQ ID NO: 1. In some embodiments, an agent interacts with a residue that is or corresponds to Gln24 of SEQ ID NO: 1. In some embodiments, an agent interacts with a residue that is or corresponds to Glu25 of SEQ ID NO: 1. In some embodiments, an agent interacts with a residue that is or corresponds to Thr26 of SEQ ID NO: 1. In some embodiments, an agent interacts with a residue that is or corresponds to Thr49 of SEQ ID NO: 1. In some embodiments, an agent interacts with a residue that is or corresponds to Met50 of SEQ ID NO: 1. In some embodiments, an agent interacts with a residue that is or corresponds to Lys51 of SEQ ID NO: 1. In some embodiments, an agent interacts with a residue that is or corresponds to Leu54 of SEQ ID NO: 1. In some embodiments, an agent interacts with a residue that is or corresponds to Phe55 of SEQ ID NO: 1. In some embodiments, an agent interacts with a residue that is or corresponds to Leu57 of SEQ ID NO: 1. In some embodiments, an agent interacts with a residue that is or corresponds to Gly58 of SEQ ID NO: 1. In some embodiments, an agent interacts with a residue that is or corresponds to Gln59 of SEQ ID NO: 1. In some embodiments, an agent interacts with a residue that is or corresponds to Ile61 of SEQ ID NO: 1. In some embodiments, an agent interacts with a residue that is or corresponds to Met62 of SEQ ID NO: 1. In some embodiments, an agent interacts with a residue that is or corresponds to Tyr67 of SEQ ID NO: 1. In some embodiments, an agent interacts with a residue that is or corresponds to Gln72 of SEQ ID NO: 1. In some embodiments, an agent interacts with a residue that is or corresponds to His73 of SEQ ID NO: 1. In some embodiments, an agent interacts with a residue that is or corresponds to Val75 of SEQ ID NO: 1. In some embodiments, an agent interacts with a residue that is or corresponds to Phe91 of SEQ ID NO: 1. In some embodiments, an agent interacts with a residue that is or corresponds to Val93 of SEQ ID NO: 1. In some embodiments, an agent interacts with a residue that is or corresponds to Lys94 of SEQ ID NO: 1. In some embodiments, an agent interacts with a residue that is or corresponds to His96 of SEQ ID NO: 1. In some embodiments, an agent interacts with a residue that is or corresponds to Arg97 of SEQ ID NO: 1. In some embodiments, an agent interacts with a residue that is or corresponds to Ile99 of SEQ ID NO: 1. In some embodiments, an agent interacts with a residue that is or corresponds to Tyr100 of SEQ ID NO: 1. In some embodiments, an agent interacts with a residue that is or corresponds to Ile103 of SEQ ID NO: 1. In some embodiments, an agent interacts with a residue that is or corresponds to Tyr104 of SEQ ID NO: 1.

### CHIP Bind Sties

In some embodiments, agents, e.g., peptides, interact with CHIP or a portion thereof. In some embodiments, agents, e.g., peptides, interact with polypeptides comprising characteristic portions and/or residues of CHIP. In some embodiments, agents, e.g., peptides, interact with polypeptides that are or comprise TPR domain of CHIP.

For example, in some embodiments, an agent interacts with one or more residues each of which independently is or corresponds to Gln27, Lys30, Glu31, Asn34, Phe37, Val38, Arg40, Tyr42, Tyr49, Val61, Thr64, Asn65, Leu68, Leu71, Lys72, Gln74, Ser93, Val94, Lys95, Phe98, Phe99, Gln102, Leu105, Glu106, Leu117, Gln127, Leu129, Asn130, Phe131, Gly132, Asp134, Ile135, Ser137, Ala138, Ile141, Ala142, and Lys145 of SEQ ID NO: 2. In some embodiments, an agent, e.g., an agent that is or comprises cluster C31 or H201, interacts with one or more residues each of which independently is or corresponds to Lys30, Asn34, Phe37, Val38, Arg40, Tyr42, Tyr49, Val61, Asn65, Leu68, Leu71, Lys72, Gln74, Ser93, Val94, Lys95, Phe98, Phe99, Gln102, Glu106, Leu129, Asn130, Phe131, Asp134, and Lys145 of SEQ ID NO: 2. In some embodiments, an agent, e.g., an agent that is or comprises cluster C32 or H203, interacts with one or more residues each of which independently is or corresponds to Asn34, Phe37, Val38, Arg40, Tyr42, Tyr49, Val61, Asn65, Leu68, Leu71, Lys72, Gln74, Val94, Lys95, Phe98, Phe99, Gln102, Leu105, Glu106, Leu117, Leu129, Asn130, Phe131, Gly132, Asp134, Ile135, Ser137, Ala138, Ile141, Ala142, and Lys145 of SEQ ID NO: 2. In some embodiments, an agent, e.g., an agent that is or comprises cluster C33 or H202, interacts with one or more residues each of which independently is or corresponds to Gln27, Lys30, Glu31, Asn34, Phe37, Val38, Val61, Thr64, Asn65, Leu68, Lys72, Val94, Lys95, Phe98, Phe99, Gln102, Leu105, Leu129, Phe131, Gly132, Asp134, Ile135, Ser137, Ala138, Ile141, and Lys145 of SEQ ID NO: 2.

In some embodiments, an agent interacts with one or more residues each of which independently is or corresponds to Gln27, Lys30, Glu31, Asn34, Phe37, Val38, Arg40, Tyr42, Tyr49, Val61, Thr64, Asn65, Leu68, Leu71, Lys72, Gln74, Ser93, Val94, Lys95, Phe98, Phe99, Gln102, Leu105, Glu106, Leu129, Asn130, Phe131, Gly132, Asp134, Ile135, Ser137, Ala138, Ile141, and Lys145 of SEQ ID NO: 2. In some embodiments, an agent, e.g., an agent that is or comprises cluster C31 or H201, interacts with one or more residues each of which independently is or corresponds to Lys30, Asn34, Phe37, Val38, Arg40, Tyr42, Tyr49, Val61, Asn65, Leu68, Leu71, Lys72, Gln74, Ser93, Val94, Lys95, Phe98, Phe99, Gln102, Leu129, Asn130, Phe131, and Asp134 of SEQ ID NO: 2. In some embodiments, an agent, e.g., an agent that is or comprises cluster C32 or H203, interacts with one or more residues each of which independently is or corresponds to Asn34, Phe37, Val38, Arg40, Tyr49, Val61, Asn65, Leu68, Leu71, Lys72, Gln74, Val94, Lys95, Phe98, Phe99, Gln102, Leu105, Glu106, Leu129, Asn130, Phe131, Asp134, Ile135, Ser137, Ala138, Ile141, and Lys145 of SEQ ID NO: 2. In some embodiments, an agent, e.g., an agent that is or comprises cluster C33 or H202, interacts with one or more residues each of which independently is or corresponds to Lys30, Gln27, Glu31, Asn34, Phe37, Val38, Val61, Thr64, Asn65, Leu68, Lys72, Lys95, Phe98, Phe99, Gln102, Leu105, Leu129, Phe131, Gly132, Asp134, Ile135, Ser137, Ala138, Ile141, and Lys145 of SEQ ID NO: 2.

In some embodiments, an agent interacts with a residue that is or corresponds to Gln27 of SEQ ID NO: 2. In some embodiments, an agent interacts with a residue that is or corresponds to Lys30 of SEQ ID NO: 2. In some embodiments, an agent interacts with a residue that is or corresponds to Glu31 of SEQ ID NO: 2. In some embodiments, an agent interacts with a residue that is or corresponds to Asn34 of SEQ ID NO: 2. In some embodiments, an agent interacts with a residue that is or corresponds to Phe37 of SEQ ID NO: 2. In some embodiments, an agent interacts with a residue that is or corresponds to Val38 of SEQ ID NO: 2. In some embodiments, an agent interacts with a residue that is or corresponds to Arg40 of SEQ ID NO: 2. In some embodiments, an agent interacts with a residue that is or corresponds to Tyr42 of SEQ ID NO: 2. In some embodiments, an agent interacts with a residue that is or corresponds to Tyr49 of SEQ ID NO: 2. In some embodiments, an agent interacts with a residue that is or corresponds to Val61 of SEQ ID NO: 2. In some embodiments, an agent interacts with a residue that is or corresponds to Thr64 of SEQ ID NO: 2. In some embodiments, an agent interacts with a residue that is or corresponds to Asn65 of SEQ ID NO: 2. In some embodiments, an agent interacts with a residue that is or corresponds to Leu68 of SEQ ID NO: 2. In some embodiments, an agent interacts with a residue that is or corresponds to Leu71 of SEQ ID NO: 2. In some embodiments, an agent interacts with a residue that is or corresponds to Lys72 of SEQ ID NO: 2. In some embodiments, an agent interacts with a residue that is or corresponds to Gln74 of SEQ ID NO: 2. In some embodiments, an agent interacts with a residue that is or corresponds to Ser93 of SEQ ID NO: 2. In some embodiments, an agent interacts with a residue that is or corresponds to Val94 of SEQ ID NO: 2. In some embodiments, an agent interacts with a residue that is or corresponds to Lys95 of SEQ ID NO: 2. In some embodiments, an agent interacts with a residue that is or corresponds to Phe98 of SEQ ID NO: 2. In some embodiments, an agent interacts with a residue that is or corresponds to Phe99 of SEQ ID NO: 2. In some embodiments, an agent interacts with a residue that is or corresponds to Gln102 of SEQ ID NO: 2. In some embodiments, an agent interacts with a residue that is or corresponds to Leu105 of SEQ ID NO: 2. In some embodiments, an agent interacts with a residue that is or corresponds to Glu106 of SEQ ID NO: 2. In some embodiments, an agent interacts with a residue that is or corresponds to Leu 117 of SEQ ID NO: 2. In some embodiments, an agent interacts with a residue that is or corresponds to Gln127 of SEQ ID NO: 2. In some embodiments, an agent interacts with a residue that is or corresponds to Leu129 of SEQ ID NO: 2. In some embodiments, an agent interacts with a residue that is or corresponds to Asn130 of SEQ ID NO: 2. In some embodiments, an agent interacts with a residue that is or corresponds to Phe131 of SEQ ID NO: 2. In some embodiments, an agent interacts with a residue that is or corresponds to Gly132 of SEQ ID NO: 2. In some embodiments, an agent interacts with a residue that is or corresponds to Asp134 of SEQ ID NO: 2. In some embodiments, an agent interacts with a residue that is or corresponds to Ile135 of SEQ ID NO: 2. In some embodiments, an agent interacts with a residue that is or corresponds to Ser137 of SEQ ID NO: 2. In some embodiments, an agent interacts with a residue that is or corresponds to Ala138 of SEQ ID NO: 2. In some embodiments, an agent interacts with a residue that is or corresponds to Ile141 of SEQ ID NO: 2. In some embodiments, an agent interacts with a residue that is or corresponds to Ala142 of SEQ ID NO: 2. In some embodiments, an agent interacts with a residue that is or corresponds to Lys145 of SEQ ID NO: 2.

### Certain Agents

Among other things, the present disclosure provides agents, e.g. peptides, that can bind to target polypeptides.

### CHIP

In some embodiments, provided agents bind to CHIP. Certain agents that can bind to CHIP are described below as examples. In some embodiments, stapled peptides comprise sequences of certain clusters, e.g., C31, C32, C33, etc. In some embodiments, stapled peptides comprise acidic residues, e.g., residues with carboxylate functionalities (e.g., X³ in cluster C31, X³²/X² in cluster C32, X¹² in cluster C33) across clusters. In some embodiments, hydrophobic residues are at i,i+4/5 positions relative to such acidic residues (e.g., D-aspartic acid residue). For example, X⁶ (e.g., dW6) in cluster C31, X⁶ (e.g., dW6) in cluster C32, and X⁸ (e.g., dA8) in cluster C33.

### Cluster C31

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C31 as described herein. In some embodiments, an agent comprises X³¹X²X³X⁴X⁵X⁶X⁷X⁸X⁹X¹⁰X¹¹X¹²X¹³X¹⁴, wherein each of X³¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³¹X²X³X⁴X⁵X⁶X⁷X⁸X⁹X¹⁰X¹¹X¹²X¹³X¹⁴,

wherein:
each of X³¹, X², X³, X⁴, X⁵, X⁶, X⁷, X⁸, X⁹, X¹, X¹¹, X¹², X¹³, and X¹⁴ is independently an amino acid residue;
X³ comprises a side chain comprising an acidic group;
X⁶ comprises a hydrophobic side chain or a side chain comprising an aromatic group;
X⁷ comprises a hydrophobic side chain or a side chain comprising an aromatic group; and
X⁸ comprises a hydrophobic side chain.

Various amino acid residues may be utilized for X³¹. For example, in some embodiments, X³¹ is D-Trp.

Various amino acid residues may be utilized for X². In some embodiments, X² is an acidic residue. In some embodiments, side chain of X² comprises —COOH or a salt form thereof. For example, in some embodiments, X² is D-Glu.

Various amino acid residues may be utilized for X³. For example, in some embodiments, X³ comprises a side chain comprising an acidic group. In some embodiments, a cluster is enriched for amino acid residues whose side chains comprising acidic groups at X³. In some embodiments, X³ comprises a side chain comprising —COOH or a salt form thereof. In some embodiments, X³ is D-Asp. In some embodiments, X³ is D-Glu.

In some embodiments, X⁴ is a residue for stapling. In some embodiments, X⁴ is stapled with an amino acid residue. In some embodiments, X⁴ is D-Cys. In some embodiments, X⁴ is not D-Cys. In some embodiments, X⁴ comprises a side chain comprising —CH═CH₂. In some embodiments, X¹¹ is a residue for stapling. In some embodiments, X¹¹ is stapled with an amino acid residue. In some embodiments, X¹¹ is D-Cys. In some embodiments, X¹¹ is not D-Cys. In some embodiments, X¹¹ comprises a side chain comprising —CH═CH₂. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

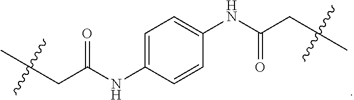 [SMILES: *CC(=O)Nc1ccc(NC(=O)C*)cc1]

Various amino acid residues may be utilized for X⁵. For example, in some embodiments, X⁵ comprises a hydrophobic side chain. In some embodiments, the side chain of X⁵ comprises an aromatic group. In some embodiments, a cluster is enriched for amino acid residues comprising an aromatic group at X⁵. In some embodiments, X⁵ comprises a side chain comprising —OH. In some embodiments, X⁵ comprises a hydrophobic side chain. In some embodiments, X⁵ is D-Tyr. In some embodiments, X⁵ is D-Phe. In some embodiments, X⁵ is D-Ala.

Various amino acid residues may be utilized for X⁶. In some embodiments, X⁶ comprises a hydrophobic side chain or a side chain comprising an aromatic group. In some embodiments, a cluster is enriched for amino acid residues whose side chains comprising aromatic groups at X⁶. In some embodiments, X⁶ comprises a hydrophobic side chain. In some embodiments, a cluster is enriched for amino acid residues comprising hydrophobic side chains at X⁶. In some embodiments, X⁶ comprises a side chain comprising an aromatic group. In some embodiments, X⁶ is D-Trp.

Various amino acid residues may be utilized for X⁷. For example, in some embodiments, the side chain of X⁷ comprises an aromatic group. In some embodiments, a cluster is enriched for amino acid residues whose side chains comprising aromatic groups at X⁷. In some embodiments, X⁷ comprises a hydrophobic side chain. In some embodiments, a cluster is enriched for amino acid residues comprising hydrophobic side chains at X⁷. In some embodiments, X⁷ is D-Phe.

Various amino acid residues may be utilized for X⁸. For example, in some embodiments, X⁸ comprises a hydrophobic side chain. In some embodiments, a cluster is enriched for amino acid residues comprising hydrophobic side chains at X⁸. In some embodiments, the side chain of X⁸ is C₁₋₆ aliphatic. In some embodiments, the side chain of X⁸ is C₁₋₆ alkyl. In some embodiments, X⁸ is D-Ala.

Various amino acid residues may be utilized for X⁹. In some embodiments, X⁹ comprises a side chain comprising an aromatic group. In some embodiments, X⁹ comprises a hydrophobic side chain. For example, in some embodiments, X⁹ is D-Trp.

Various amino acid residues may be utilized for X¹⁰. For example, in some embodiments, X¹⁰ comprises a hydrophobic side chain. In some embodiments, a cluster is enriched for amino acid residues comprising a hydrophobic side chain at X¹⁰. In some embodiments, the side chain of X¹⁰ is C₁₋₆ aliphatic. In some embodiments, the side chain of X¹⁰ is C₁₋₆ alkyl. In some embodiments, X¹⁰ is D-Met. In some embodiments, X¹⁰ is D-Ala. In some embodiments, X¹⁰ comprises a side chain comprising a polar group. In some embodiments, X¹⁰ comprises a side chain comprising —OH. In some embodiments, X¹⁰ is D-Ser.

Various amino acid residues may be utilized for X¹². For example, in some embodiments, X¹² comprises a side chain comprising a polar group. In some embodiments, X¹² comprises a side chain comprising —OH. In some embodiments, X¹² is D-Gln. In some embodiments, X¹² is D-Tyr.

Various amino acid residues may be utilized for X¹³. For example, in some embodiments, the side chain of X¹³ comprises an aromatic group. In some embodiments, X¹³ comprises a side chain comprising a basic group. In some embodiments, X¹³ is D-His. In some embodiments, X¹³ is D-Asn.

Various amino acid residues may be utilized for X¹⁴. For example, in some embodiments, X¹⁴ comprises a hydrophobic side chain. In some embodiments, X¹⁴ is D-Ile.

In some embodiments, an agent is or comprises H201. In some embodiments, X³ (e.g., dD), X⁶ (e.g., dW), X⁷ (e.g., dF), X⁹ (e.g., dW), X¹⁰ (e.g., dA), and/or X¹⁴ (e.g., dI) interact with CHIP. For example, in some embodiments, dD4, dW7, dF8, dW10, dA11, and/or dI15 of H201 interact with CHIP.

### Cluster C32

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C32 as described herein. In some embodiments, an agent comprises X³²X²X³X⁴X⁵X⁶X⁷X⁸X⁹X¹⁰X¹¹X¹²X¹³X¹⁴, wherein each of X³², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³²X²X³X⁴X⁵X⁶X⁷X⁸X⁹X¹⁰X¹¹X¹²X¹³X¹⁴,

wherein:
each of X³², X², X³, X⁴, X⁵, X⁶, X⁷, X⁸, X⁹, X¹⁰, X¹¹, X¹², X¹³, and X¹⁴ is independently an amino acid residue;
X² comprises a side chain comprising a polar group;
X³ comprises a hydrophobic side chain; and
X⁶ comprises a hydrophobic side chain or a side chain comprising an aromatic group.

Various amino acid residues may be utilized for X³². For example, in some embodiments, X³² comprises a side chain comprising an acidic group. In some embodiments, X³² comprises a side chain comprising —COOH or a salt form thereof. In some embodiments, the side chain of X³² comprises a polar group. In some embodiments, X³² comprises a side chain comprising an aromatic group. In some embodiments, X³² is D-Asp. In some embodiments, X³² is D-Tyr. In some embodiments, X³² is D-His. In some embodiments, X³² is D-Leu.

Various amino acid residues may be utilized for X². For example, in some embodiments, X² comprises a side chain comprising an acidic group. In some embodiments, a cluster is enriched for amino acid residues whose side chains comprising acidic groups at X². In some embodiments, X² comprises a side chain comprising —COOH or a salt thereof. In some embodiments, X² is D-Asp. In some embodiments, X² is D-Glu. In some embodiments, X² comprises a side chain comprising a polar group. In some embodiments, X² comprises a side chain comprising —OH. In some embodiments, X² is D-Thr.

Various amino acid residues may be utilized for X³. For example, in some embodiments, X³ comprises a hydrophobic side chain. In some embodiments, a cluster is enriched for amino acid residues comprising a hydrophobic side chain at X³. In some embodiments, the side chain of X³ is C₁₋₆ aliphatic. In some embodiments, the side chain of X³ is C₁₋₆ alkyl. In some embodiments, X³ is D-Leu. In some embodiments, X³ is D-Met. In some embodiments, X³ is D-Ile.

In some embodiments, X⁴ is a residue for stapling. In some embodiments, X⁴ is stapled with an amino acid residue. In some embodiments, X⁴ is D-Cys. In some embodiments, X⁴ is not D-Cys. In some embodiments, X⁴ comprises a side chain comprising —CH═CH₂. In some embodiments, X¹¹ is a residue for stapling. In some embodiments, X¹¹ is stapled with an amino acid residue. In some embodiments, X¹¹ is D-Cys. In some embodiments, X¹¹ is not D-Cys. In some embodiments, X¹¹ comprises a side chain comprising —CH═CH₂. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

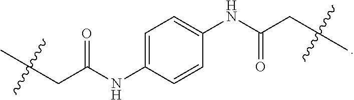 [SMILES: *CC(=O)Nc1ccc(NC(=O)C*)cc1]

Various amino acid residues may be utilized for X⁵. For example, in some embodiments, X⁵ comprises a hydrophobic side chain. In some embodiments, X⁵ comprises a side chain comprising an aromatic group. In some embodiments, X⁵ comprises a side chain comprising a polar group, e.g., —OH. In some embodiments, X⁵ comprises a side chain comprising an acidic group, e.g., —COOH or a salt form thereof. In some embodiments, X⁵ is D-Tyr. In some embodiments, X⁵ is D-Leu.

Various amino acid residues may be utilized for X⁶. For example, in some embodiments, X⁶ comprises a hydrophobic side chain. In some embodiments, X⁶ comprises a side chain comprising an aromatic group. In some embodiments, a cluster is enriched for amino acid residues comprising a hydrophobic side chain at X⁶. In some embodiments, a cluster is enriched for amino acid residues comprising an aromatic group at X⁶. In some embodiments, X⁶ is D-Trp.

Various amino acid residues may be utilized for X⁷. For example, in some embodiments, X⁷ comprises a hydrophobic side chain. In some embodiments, a cluster is enriched for amino acid residues comprising a hydrophobic side chain at X⁷. In some embodiments, the side chain of X⁷ is C₁₋₆ aliphatic. In some embodiments, the side chain of X⁷ is C₁₋₆ alkyl. In some embodiments, X⁷ is D-Ala. In some embodiments, X⁷ is D-Ser.

Various amino acid residues may be utilized for X⁸. For example, in some embodiments, X⁸ comprises a side chain comprising an acidic group. In some embodiments, X⁸ comprises a side chain comprising —COOH or a salt form thereof. In some embodiments, X⁸ comprises a side chain comprising a polar group, e.g., —OH. In some embodiments, X⁸ is D-Asp. In some embodiments, X⁸ is D-Glu. In some embodiments, X⁸ is D-Asn. In some embodiments, X⁸ is D-Ser.

Various amino acid residues may be utilized for X⁹. For example, in some embodiments, X⁹ comprises a hydrophobic side chain. In some embodiments, the side chain of X⁹ is C₁₋₆ aliphatic. In some embodiments, the side chain of X⁹ is C₁₋₆ alkyl. In some embodiments, X⁹ is D-Asn. In some embodiments, X⁹ is D-Ala. In some embodiments, X⁹ is D-Leu.

Various amino acid residues may be utilized for X¹⁰. For example, in some embodiments, X¹⁰ comprises a side chain comprising an acidic group. In some embodiments, a cluster is enriched for amino acid residues whose side chains comprise acidic groups at X¹⁰. In some embodiments, X¹⁰ comprises a side chain comprising —COOH or a salt form thereof. In some embodiments, X¹⁰ is D-Glu. In some embodiments, X¹⁰ comprises a side chain comprising a polar group. In some embodiments, X¹⁰ comprises a side chain comprising —OH. In some embodiments, X¹⁰ comprises a side chain comprising an aromatic group. In some embodiments, X¹⁰ is D-Tyr. In some embodiments, X¹⁰ is D-His.

Various amino acid residues may be utilized for X¹². For example, in some embodiments, X¹² comprises a hydrophobic side chain. In some embodiments, the side chain of X¹² is C₁₋₆ aliphatic. In some embodiments, the side chain of X¹² is C₁₋₆ alkyl. In some embodiments, X¹² is D-Ile. In some embodiments, X¹² is D-Met. In some embodiments, X¹² comprises a side chain comprising a basic group. In some embodiments, X¹² is D-Arg.

Various amino acid residues may be utilized for X¹³. For example, in some embodiments, X¹³ comprises a hydrophobic side chain. In some embodiments, the side chain of X¹³ is C₁₋₆ aliphatic. In some embodiments, the side chain of X¹³ is C₁₋₆ alkyl. In some embodiments, X¹³ is D-Val. In some embodiments, X¹³ is D-Leu. In some embodiments, X³ comprises a side chain comprising an aromatic group. In some embodiments, X¹³ is D-Phe. In some embodiments, X¹³ is D-Tyr.

Various amino acid residues may be utilized for X¹⁴. For example, in some embodiments, X¹⁴ comprises a side chain comprising a polar group. In some embodiments, X¹⁴ comprises a side chain comprising —OH. In some embodiments, X¹⁴ is D-Ser. In some embodiments, X¹⁴ comprises a side chain comprising a basic group. In some embodiments, X¹⁴ is D-Arg. In some embodiments, X¹⁴ is D-Ala.

In some embodiments, an agent is or comprises H203. In some embodiments, X² (e.g., dE), X³ (e.g., dM), X⁵ (e.g., dY), X⁶ (e.g., dW), X⁷ (e.g., dA), X¹⁰ (e.g., dY), and/or X¹³ (e.g., dY) interact with CHIP. For example, in some embodiments, dE3, dM4, dY6, dW7, dA8, dY11, and/or dY14 of H203 interact with CHIP.

### Cluster C33

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C33 as described herein. In some embodiments, an agent comprises X³³X²X³X⁴X⁵X⁶X⁷X⁸X⁹X¹⁰X¹¹X¹²X¹³X¹⁴, wherein each of X³³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X³³X²X³X⁴X⁵X⁶X⁷X⁸X⁹X¹⁰X¹¹X¹²X¹³X¹⁴,

wherein:
each of X³³, X², X³, X⁴, X⁵, X⁶, X⁷, X⁸, X⁹, X¹, X¹¹, X¹², X¹³, and X¹⁴ is independently an amino acid residue;
X³³ comprises a hydrophobic side chain or a side chain comprising an aromatic group; and
X⁵ comprises a hydrophobic side chain or a side chain comprising an aromatic group, or a side chain comprising a polar group.

Various amino acid residues may be utilized for X³³. For example, in some embodiments, X³³ comprises a hydrophobic side chain. In some embodiments, X³³ comprises a side chain comprising an aromatic group. In some embodiments, a cluster is enriched for amino acid residues comprising a hydrophobic side chain at X³³. In some embodiments, a cluster is enriched for amino acid residues comprising an aromatic group at X³³. In some embodiments, X³³ is D-Trp.

Various amino acid residues may be utilized for X². For example, in some embodiments, X² comprises a hydrophobic side chain. In some embodiments, X² comprises a side chain comprising an aromatic group. In some embodiments, X² comprises a side chain comprising a polar group. In some embodiments, X² comprises a side chain comprising —OH. In some embodiments, X² is D-Phe. In some embodiments, X² is D-Trp. In some embodiments, X² is D-Tyr. In some embodiments, X² is D-Thr.

Various amino acid residues may be utilized for X³. For example, in some embodiments, X³ comprises a side chain comprising a polar group. In some embodiments, X³ comprises a side chain comprising —OH. In some embodiments, X³ comprises a side chain comprising an acidic group. In some embodiments, X³ comprises a side chain comprising —COOH or a salt form thereof. In some embodiments, X³ is D-Glu. In some embodiments, X³ is D-Thr. In some embodiments, X³ is D-Ser. In some embodiments, X³ is D-Asp. In some embodiments, X³ is D-Leu.

In some embodiments, X⁴ is a residue for stapling. In some embodiments, X⁴ is stapled with an amino acid residue. In some embodiments, X⁴ is D-Cys. In some embodiments, X⁴ is not D-Cys. In some embodiments, X⁴ comprises a side chain comprising —CH═CH₂. In some embodiments, X¹¹ is a residue for stapling. In some embodiments, X¹¹ is stapled with an amino acid residue. In some embodiments, X¹¹ is D-Cys. In some embodiments, X¹¹ is not D-Cys. In some embodiments, X¹¹ comprises a side chain comprising —CH═CH₂. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

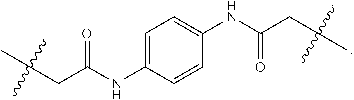 [SMILES: *CC(=O)Nc1ccc(NC(=O)C*)cc1]

Various amino acid residues may be utilized for X⁵. For example, in some embodiments, X⁵ comprises a hydrophobic side chain. In some embodiments, a cluster is enriched for amino acid residues comprising a hydrophobic side chain at X⁵. In some embodiments, the side chain of X⁵ is C₁₋₆ aliphatic. In some embodiments, the side chain of X⁵ is C₁₋₆ alkyl. In some embodiments, X⁵ is D-Leu. In some embodiments, the side chain of X⁵ comprises an aromatic group. In some embodiments, a cluster is enriched for amino acid residues comprising a side chain comprising an aromatic group at X⁵. In some embodiments, X⁵ is D-Phe. In some embodiments, X⁵ is D-Tyr. In some embodiments, X⁵ is D-Trp.

Various amino acid residues may be utilized for X⁶. In some embodiments, the side chain of X⁶ comprises a basic group. In some embodiments, the side chain of X⁶ comprises an acidic group, e.g., —COOH or a salt form thereof. In some embodiments, the side chain of X⁶ comprises a polar group, e.g., —OH. In some embodiments, X⁶ comprises a hydrophobic side chain. In some embodiments, X⁶ is D-Ser.

Various amino acid residues may be utilized for X⁷. In some embodiments, the side chain of X⁷ comprises a basic group. In some embodiments, the side chain of X⁷ comprises an acidic group, e.g., —COOH or a salt form thereof. In some embodiments, the side chain of X⁷ comprises a polar group, e.g., —OH, —C(O)NH₂, etc. In some embodiments, X⁷ comprises a hydrophobic side chain. In some embodiments, X⁷ is D-Gln.

Various amino acid residues may be utilized for X⁸. For example, in some embodiments, X⁸ comprises a hydrophobic side chain. In some embodiments, a cluster is enriched for amino acid residues comprising a hydrophobic side chain at X⁸. In some embodiments, the side chain of X⁸ is C₁₋₆ aliphatic. In some embodiments, the side chain of X⁸ is C₁₋₆ alkyl. In some embodiments, X⁸ is D-Ala. In some embodiments, X⁸ is D-Ser.

Various amino acid residues may be utilized for X⁹. For example, in some embodiments, X⁹ comprises a side chain comprising an acidic group. In some embodiments, X⁹ comprises a side chain comprising —COOH or a salt form thereof. In some embodiments, X⁹ is D-Asp.

Various amino acid residues may be utilized for X¹⁰. For example, in some embodiments, X¹⁰ comprises a side chain comprising an acidic group. In some embodiments, X¹⁰ comprises a side chain comprising —COOH or a salt form thereof. In some embodiments, X¹⁰ is D-Asp.

Various amino acid residues may be utilized for X¹². For example, in some embodiments, X¹² comprises a side chain comprising an acidic group. In some embodiments, a cluster is enriched for amino acid residues whose side chains comprise acidic groups at X¹². In some embodiments, X¹² comprises a side chain comprising —COOH or a salt thereof. In some embodiments, X¹² comprises a side chain comprising a polar group, e.g., —OH, —C(O)NH₂, etc. In some embodiments, X¹² is D-Asp. In some embodiments, X¹² is D-Glu. In some embodiments, X¹² is D-Asn.

Various amino acid residues may be utilized for X¹³. For example, in some embodiments, the side chain of X¹³ comprises a side chain comprising a polar group, e.g., —OH, —C(O)NH₂, etc. In some embodiments, the side chain of X¹³ comprises a side chain comprising an acidic group. In some embodiments, the side chain of X¹³ comprises a side chain comprising —COOH or a salt form thereof. In some embodiments, X¹³ is D-Phe.

Various amino acid residues may be utilized for X¹⁴. For example, in some embodiments, the side chain of X¹⁴ comprises a hydrophobic side chain. In some embodiments, the side chain of X¹⁴ comprises a side chain comprising a basic group. In some embodiments, the side chain of X¹⁴ comprises a side chain comprising a polar group. In some embodiments, X¹⁴ is D-Arg.

In some embodiments, an agent is or comprises H202. In some embodiments, X³³ (e.g., dW), X² (e.g., dW), X⁵ (e.g., dL), X⁸ (e.g., dA), X⁹ (e.g., dD), and/or X¹² (e.g., dD) interact with CHIP. For example, in some embodiments, dW2, dW3, dL6, dA9, dD10, and/or dD13 interact with CHIP. In some embodiments, interacting residues of certain properties are presented in a reverse direction (e.g., from N to C direction aromatic/hydrophobic residues to acidic residues) relative to cluster 31 and 32 (e.g., from N to C direction acidic residues to aromatic/hydrophobic residues).

### MDM2

In some embodiments, provided agents bind to MDM2. Certain agents that can bind to MDM2 are described below as examples. In some embodiments, stapled peptides comprise sequences of certain clusters, e.g., C41, C42, C43, etc. In some embodiments, hydrophobic residues are on the same α-helical face (e.g., i,i+4/5 and i,i+8/9 relative to X² of cluster C41, X⁴² of cluster C42, or X⁴³ of cluster C43).

### Cluster C41

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C41 as described herein. In some embodiments, an agent comprises X⁴¹X²X³X⁴X⁵X⁶X⁷X⁸X⁹X¹⁰X¹¹X¹²X¹³X¹⁴, wherein each of X⁴¹, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴¹X²X³X⁴X⁵X⁶X⁷X⁸X⁹X¹⁰X¹¹X¹²X¹³X¹⁴,

wherein:
each of X⁴¹, X², X³, X⁴, X⁵, X⁶, X⁷, X⁸, X⁹, X¹⁰, X¹¹, X¹², X¹³, and X¹⁴ is independently an amino acid residue;
X² comprises a side chain comprising an aromatic group or a hydrophobic side chain; and
X⁶ or X⁷ comprises a hydrophobic side chain or a side chain comprising an aromatic group.

Various amino acid residues may be utilized for X⁴¹. For example, in some embodiments, the side chain of X⁴¹ comprises a hydrophobic side chain. In some embodiments, the side chain of X⁴¹ comprises a side chain comprising a basic group. the side chain of X⁴¹ comprises a side chain comprising an aromatic group. In some embodiments, X⁴¹ is D-His.

Various amino acid residues may be utilized for X². For example, in some embodiments, X² comprises a hydrophobic side chain. In some embodiments, a cluster is enriched for amino acid residues comprise hydrophobic side chains at X². In some embodiments, X² comprises a side chain comprising an aromatic group. In some embodiments, a cluster is enriched for amino acid residues that comprise side chains comprising aromatic groups. In some embodiments, X² is D-Trp.

Various amino acid residues may be utilized for X³. For example, in some embodiments, X³ comprises a hydrophobic side chain. In some embodiments, a cluster is enriched for amino acid residues comprise hydrophobic side chains at X³. In some embodiments, X³ comprises a side chain comprising an aromatic group. In some embodiments, a cluster is enriched for amino acid residues that comprise side chains comprising aromatic groups. In some embodiments, X³ comprises a side chain comprising a polar group, e.g., —OH. In some embodiments, X³ is D-Tyr.

In some embodiments, X⁴ is a residue for stapling. In some embodiments, X⁴ is stapled with an amino acid residue. In some embodiments, X⁴ is D-Cys. In some embodiments, X⁴ is not D-Cys. In some embodiments, X⁴ comprises a side chain comprising —CH═CH₂. In some embodiments, X¹¹ is a residue for stapling. In some embodiments, X¹¹ is stapled with an amino acid residue. In some embodiments, X¹¹ is D-Cys. In some embodiments, X¹¹ is not D-Cys. In some embodiments, X¹¹ comprises a side chain comprising —CH═CH₂. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

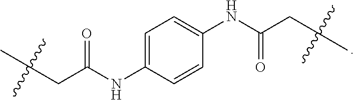 [SMILES: *CC(=O)Nc1ccc(NC(=O)C*)cc1]

Various amino acid residues may be utilized for X⁵. For example, in some embodiments, X⁵ comprises a side chain comprising an acidic group. In some embodiments, X⁵ comprises a side chain comprising —COOH or a salt form thereof. In some embodiments, X⁵ is D-Asp. In some embodiments, X⁵ is D-Glu.

Various amino acid residues may be utilized for X⁶. For example, in some embodiments, X⁶ comprises a hydrophobic side chain. In some embodiments, a cluster is enriched for amino acid residues comprise hydrophobic side chains at X⁶. In some embodiments, the side chain of X⁶ is C₁₋₆ aliphatic. In some embodiments, the side chain of X⁶ is C₁₋₆ alkyl. In some embodiments, X⁶ comprises a side chain comprising an aromatic group. In some embodiments, X⁶ is D-Leu. In some embodiments, X⁶ is D-Ile. In some embodiments, X⁶ is D-Phe. In some embodiments, X⁶ is D-Met. In some embodiments, X⁶ is D-Val.

Various amino acid residues may be utilized for X⁷. For example, in some embodiments, X⁷ comprises a hydrophobic side chain. In some embodiments, a cluster is enriched for amino acid residues comprise hydrophobic side chains at X⁷. In some embodiments, the side chain of X⁷ is C₁₋₆ aliphatic. In some embodiments, the side chain of X⁷ is C₁₋₆ alkyl. In some embodiments, X⁷ is D-Ala.

Various amino acid residues may be utilized for X⁸. For example, in some embodiments, X⁸ comprises a hydrophobic side chain. In some embodiments, a cluster is enriched for amino acid residues comprise hydrophobic side chains at X⁸. In some embodiments, the side chain of X⁸ is C₁₋₆ aliphatic. In some embodiments, the side chain of X⁸ is C₁₋₆ alkyl. In some embodiments, X⁸ is D-Ala.

Various amino acid residues may be utilized for X⁹. For example, in some embodiments, X⁹ comprises a hydrophobic side chain. In some embodiments, X⁹ comprises a side chain comprising a polar group. In some embodiments, X⁹ is D-Gln.

Various amino acid residues may be utilized for X¹⁰. For example, in some embodiments, X¹⁰ comprises a hydrophobic side chain. In some embodiments, X¹⁰ comprises a side chain comprising a polar group. In some embodiments, X¹⁰ is D-Met. In some embodiments, X¹⁰ is D-Nle. In some embodiments, X¹⁰ is D-Val.

Various amino acid residues may be utilized for X¹². For example, in some embodiments, X¹² comprises a hydrophobic side chain. In some embodiments, X¹² comprises a side chain comprising a polar group. In some embodiments, X¹² is D-Asn.

Various amino acid residues may be utilized for X¹³. For example, in some embodiments, X¹³ comprises a hydrophobic side chain. In some embodiments, X¹³ comprises a side chain comprising a polar group. In some embodiments, X¹³ comprises a side chain comprising an acidic group. In some embodiments, X¹³ comprises a side chain comprising an aromatic group. In some embodiments, X¹³ is D-Glu. In some embodiments, X¹³ is D-Asp. In some embodiments, X¹³ is D-Phe.

Various amino acid residues may be utilized for X¹⁴. For example, in some embodiments, X¹⁴ comprises a hydrophobic side chain. In some embodiments, X¹⁴ comprises a side chain comprising a polar group. In some embodiments, X¹⁴ is D-Asn. In some embodiments, X¹⁴ is D-Ser.

In some embodiments, an agent is or comprises H101. In some embodiments, X² (e.g., dW), X³ (e.g., dY), X⁶ (e.g., dF), X⁹ (e.g., dQ), X¹⁰ (e.g., dV), and/or X¹³ (e.g., dF) interact with MDM2. For example, in some embodiments, dW5, dY6, dF9, dQ12, dV13, and/or dF16 of H101 interact with MDM2.

### Cluster C42

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C42 as described herein. In some embodiments, an agent comprises X⁴²X²X³X⁴X⁵X⁶X⁷X⁸X⁹X¹⁰X¹¹X¹²X¹³X¹⁴, wherein each of X⁴², X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴²X²X³X⁴X⁵X⁶X⁷X⁸X⁹X¹⁰X¹¹X¹²X¹³X¹⁴,

wherein:
each of X⁴², X², X³, X⁴, X⁵, X⁶, X⁷, X⁸, X⁹, X¹⁰, X¹¹, X¹², X¹³, and X¹⁴ is independently an amino acid residue;
X⁴² comprises a side chain comprising an aromatic group or a hydrophobic side chain; and
X⁶ or X⁷ comprises a hydrophobic side chain or a side chain comprising an aromatic group.

Various amino acid residues may be utilized for X⁴². For example, in some embodiments, X⁴² comprises a hydrophobic side chain. In some embodiments, a cluster is enriched for amino acid residues comprising hydrophobic side chains at X⁴². In some embodiments, X⁴² comprises a side chain comprising an aromatic group. In some embodiments, a cluster is enriched for amino acid residues that comprise side chains comprising aromatic groups at X⁴². In some embodiments, X⁴² is D-Trp.

Various amino acid residues may be utilized for X². For example, in some embodiments, X² comprises a side chain comprising an acidic group. In some embodiments, X² comprises a side chain comprising —COOH or a salt form thereof. In some embodiments, X² comprises a side chain comprising a polar group, e.g., —OH, —C(O)NH₂, etc. In some embodiments, X² is D-Asp. In some embodiments, X² is D-Glu. In some embodiments, X² is D-Asn.

Various amino acid residues may be utilized for X³. For example, in some embodiments, X³ comprises a hydrophobic side chain. In some embodiments, X³ comprises a side chain comprising an aromatic group. In some embodiments, X³ comprises a side chain comprising a polar group. In some embodiments, X³ comprises a side chain comprising a basic group. In some embodiments, X³ is D-His.

In some embodiments, X⁴ is a residue for stapling. In some embodiments, X⁴ is stapled with an amino acid residue. In some embodiments, X⁴ is D-Cys. In some embodiments, X⁴ is not D-Cys. In some embodiments, X⁴ comprises a side chain comprising —CH═CH₂. In some embodiments, X¹¹ is a residue for stapling. In some embodiments, X¹¹ is stapled with an amino acid residue. In some embodiments, X¹¹ is D-Cys. In some embodiments, X¹¹ is not D-Cys. In some embodiments, X¹¹ comprises a side chain comprising —CH═CH₂. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

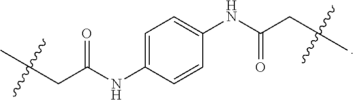 [SMILES: *CC(=O)Nc1ccc(NC(=O)C*)cc1]

Various amino acid residues may be utilized for X⁵. For example, in some embodiments, X⁵ comprises a side chain comprising an acidic group. In some embodiments, X⁵ comprises a side chain comprising —COOH or a salt form thereof. In some embodiments, X⁵ comprises a hydrophobic side chain. In some embodiments, X⁵ comprises a side chain comprising a polar group. In some embodiments, X⁵ is D-Asp. In some embodiments, X⁵ is D-Glu.

Various amino acid residues may be utilized for X⁶. For example, in some embodiments, X⁶ comprises a hydrophobic side chain. In some embodiments, a cluster is enriched for amino acid residues that comprise hydrophobic side chains at X⁶. In some embodiments, the side chain of X⁶ is C₁₋₆ aliphatic. In some embodiments, the side chain of X⁶ is C₁₋₆ alkyl. In some embodiments, X⁶ comprises a side chain comprising an aromatic group. In some embodiments, a cluster is enriched for amino acid residues comprising aromatic groups at X⁶. In some embodiments, X⁶ is D-Tyr. In some embodiments, X⁶ is D-Phe. In some embodiments, X⁶ is D-Trp.

Various amino acid residues may be utilized for X⁷. For example, in some embodiments, X⁷ comprises a hydrophobic side chain. In some embodiments, a cluster is enriched for amino acid residues comprise hydrophobic side chains at X⁷. In some embodiments, the side chain of X⁷ is C₁₋₆ aliphatic. In some embodiments, the side chain of X⁷ is C₁₋₆ alkyl. In some embodiments, X⁷ is D-Ala.

Various amino acid residues may be utilized for X⁸. For example, in some embodiments, X⁸ comprises a hydrophobic side chain. In some embodiments, a cluster is enriched for amino acid residues comprise hydrophobic side chains at X⁸. In some embodiments, the side chain of X⁸ is C₁₋₆ aliphatic. In some embodiments, the side chain of X⁸ is C₁₋₆ alkyl. In some embodiments, X⁸ is D-Ala.

Various amino acid residues may be utilized for X⁹. For example, in some embodiments, X⁹ comprises a hydrophobic side chain. In some embodiments, X⁹ comprises a side chain comprising a polar group. In some embodiments, X⁹ comprises a side chain comprising an aromatic group. In some embodiments, X⁹ comprises a side chain comprising an acidic group. In some embodiments, X⁹ comprise a side chain comprising —COOH or a salt form thereof. In some embodiments, X⁹ is D-Phe.

Various amino acid residues may be utilized for X¹⁰. For example, in some embodiments, X¹⁰ comprises a hydrophobic side chain. In some embodiments, a cluster is enriched for amino acid residues that comprise hydrophobic side chains at X¹⁰. In some embodiments, the side chain of X¹⁰ is C₁₋₆ aliphatic. In some embodiments, the side chain of X¹⁰ is C₁₋₆ alkyl. In some embodiments, X¹⁰ comprises a side chain comprising a polar group. In some embodiments, X¹⁰ is D-Ile. In some embodiments, X¹⁰ is D-Val. In some embodiments, X¹⁰ is D-Leu. In some embodiments, X¹⁰ is D-Ala.

Various amino acid residues may be utilized for X¹². For example, in some embodiments, X¹² comprises a hydrophobic side chain. In some embodiments, X¹² comprises a side chain comprising a polar group. In some embodiments, X¹² comprises a side chain comprising an acidic group. In some embodiments, X¹² comprises a side chain comprising —COOH or a salt form thereof. In some embodiments, X¹² is D-Ser.

Various amino acid residues may be utilized for X¹³. For example, in some embodiments, X¹³ comprises a hydrophobic side chain. In some embodiments, X¹³ comprises a side chain comprising a polar group. In some embodiments, X¹³ comprises a side chain comprising an acidic group. In some embodiments, X¹³ comprises a side chain comprising —COOH or a salt form thereof. In some embodiments, X¹³ comprises a side chain comprising an aromatic group. In some embodiments, X¹³ is D-Glu. In some embodiments, X¹³ is D-Asp.

Various amino acid residues may be utilized for X¹⁴. For example, in some embodiments, X¹⁴ comprises a hydrophobic side chain. In some embodiments, X¹⁴ comprises a side chain comprising a polar group. In some embodiments, X¹⁴ is D-Val.

In some embodiments, an agent is or comprises H102. In some embodiments, X⁴² (e.g. dW), X³ (e.g., dH), X⁶ (e.g., dY), X⁹ (e.g., dF), X¹⁰ (e.g., dV), X¹³ (e.g., dE) and/or X¹⁴ (e.g., dV) interact with MDM2. For example, in some embodiments, dW4, dH6, dY9, dF12, dV13, dE16, and/or dV17 in H102 interact with MDM2.

### Cluster C43

In some embodiments, the present disclosure provides agents comprising one or more (e.g., 1-14, 1-10, 1-5, 5-10, 5-9, 1, 2, 3, 4, 5, 6, 7, 8, 9, 10, 11, 12, 13, or 14) residues of cluster C43 as described herein. In some embodiments, an agent comprises X⁴³X²X³X⁴X⁵X⁶X⁷X⁸X⁹X¹⁰X¹¹X¹²X¹³X¹⁴, wherein each of X⁴³, X², X³, X⁴, X⁵, X⁶, X⁷, X⁸, X⁹, X¹⁰, X¹¹, X¹², X¹³, and X¹⁴ is independently an amino acid residue.

In some embodiments, the present disclosure provides an agent comprising

X⁴³X²X³X⁴X⁵X⁶X⁷X⁸X⁹X¹⁰X¹¹X¹²X¹³X¹⁴,

wherein:
each of X⁴³, X², X³, X⁴, X⁵, X⁶, X⁷, X⁸, X⁹, X¹, X¹¹, X¹², X¹³, and X¹⁴ is independently an amino acid residue;
X⁴³ comprises a side chain comprising an aromatic group or a hydrophobic side chain; and
X⁵ comprises a hydrophobic side chain.

Various amino acid residues may be utilized for X⁴³. For example, in some embodiments, X⁴³ comprises a hydrophobic side chain. In some embodiments, a cluster is enriched for amino acid residues comprising hydrophobic side chains at X⁴³. In some embodiments, X⁴³ comprises a side chain comprising an aromatic group. In some embodiments, a cluster is enriched for amino acid residues that comprise side chains comprising aromatic groups at X⁴³. In some embodiments, X⁴³ is D-Trp.

Various amino acid residues may be utilized for X². For example, in some embodiments, X² comprises a hydrophobic side chain. In some embodiments, X² comprises a side chain comprising an aromatic group. In some embodiments, X² comprises a side chain comprising a polar group, e.g., —OH, —C(O)NH₂, etc. In some embodiments, X² is D-Tyr. In some embodiments, X² is D-Phe.

Various amino acid residues may be utilized for X³. For example, in some embodiments, X³ comprises a side chain comprising an acidic group. In some embodiments, X³ comprises a side chain comprising —COOH or a salt form thereof. In some embodiments, X³ comprises a hydrophobic side chain. In some embodiments, X³ comprises a side chain comprising an aromatic group. In some embodiments, X³ comprises a side chain comprising a polar group. In some embodiments, X³ comprises a side chain comprising a basic group. In some embodiments, X³ is D-Asp. In some embodiments, X³ is D-Glu. In some embodiments, X³ is D-Thr. In some embodiments, X³ is D-Asn.

In some embodiments, X⁴ is a residue for stapling. In some embodiments, X⁴ is stapled with an amino acid residue. In some embodiments, X⁴ is D-Cys. In some embodiments, X⁴ is not D-Cys. In some embodiments, X⁴ comprises a side chain comprising —CH═CH₂. In some embodiments, X¹¹ is a residue for stapling. In some embodiments, X¹¹ is stapled with an amino acid residue. In some embodiments, X¹¹ is D-Cys. In some embodiments, X¹¹ is not D-Cys. In some embodiments, X¹¹ comprises a side chain comprising —CH═CH₂. In some embodiments, X⁴ is stapled with X¹¹. Various staples can be utilized in accordance with the present disclosure. In some embodiments, a staple is L^{s} as described herein. In some embodiments, X⁴ and X¹¹ are cysteine residues stapled through —SH. In some embodiments, the two —SH groups are linked through L^{s2} as described herein. In some embodiments, L^{s2} is

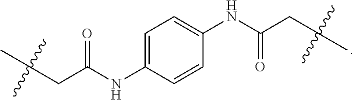 [SMILES: *CC(=O)Nc1ccc(NC(=O)C*)cc1]

Various amino acid residues may be utilized for X⁵. For example, in some embodiments, X⁵ comprises a hydrophobic side chain. In some embodiments, the side chain of X⁵ is C₁₋₆ aliphatic. In some embodiments, the side chain of X⁵ is C₁₋₆ alkyl. In some embodiments, a cluster is enriched for amino acid residues comprising hydrophobic side chains at X⁵. In some embodiments, X⁵ comprises a side chain comprising an aromatic group. In some embodiments, X⁵ comprises a side chain comprising a polar group. In some embodiments, X⁵ is D-Met. In some embodiments, X⁵ is D-Nle. In some embodiments, X⁵ is D-Leu. In some embodiments, X⁵ is D-Ile. In some embodiments, X⁵ is D-Val.

Various amino acid residues may be utilized for X⁶. For example, in some embodiments, X⁶ comprises a side chain comprising an acidic group. In some embodiments, X⁶ comprises a side chain comprising —COOH or a salt form thereof. In some embodiments, X⁶ comprises a side chain comprising a polar group, e.g., —OH, —C(O)NH₂, etc. In some embodiments, X⁶ comprises a side chain comprising a basic group. In some embodiments, X⁶ is D-Asp. In some embodiments, X⁶ is D-Glu. In some embodiments, X⁶ is D-Gln.

Various amino acid residues may be utilized for X⁷. For example, in some embodiments, X⁷ comprises a hydrophobic side chain. In some embodiments, a cluster is enriched for amino acid residues comprise hydrophobic side chains at X⁷. In some embodiments, the side chain of X⁷ is C₁₋₆ aliphatic. In some embodiments, the side chain of X⁷ is C₁₋₆ alkyl. In some embodiments, X⁷ is D-Ala.

Various amino acid residues may be utilized for X⁸. For example, in some embodiments, X⁸ comprises a hydrophobic side chain. In some embodiments, a cluster is enriched for amino acid residues comprise hydrophobic side chains at X⁸. In some embodiments, the side chain of X⁸ is C₁₋₆ aliphatic. In some embodiments, the side chain of X⁸ is C₁₋₆ alkyl. In some embodiments, X⁸ is D-Ala.

Various amino acid residues may be utilized for X⁹. For example, in some embodiments, X⁹ comprises a hydrophobic side chain. In some embodiments, the side chain of X⁹ is C₁₋₆ aliphatic. In some embodiments, the side chain of X⁹ is C₁₋₆ alkyl. In some embodiments, a cluster is enriched for amino acid residues comprising hydrophobic side chains at X⁹. In some embodiments, X⁹ comprises a side chain comprising a polar group. In some embodiments, X⁹ comprises a side chain comprising an aromatic group. In some embodiments, X⁹ is D-Leu. In some embodiments, X⁹ is D-Met. In some embodiments, X⁹ is D-Nle. In some embodiments, X⁹ is D-Ile. In some embodiments, X⁹ is D-Val. In some embodiments, X⁹ is D-Ala.

Various amino acid residues may be utilized for X¹⁰. For example, in some embodiments, X¹⁰ comprises a hydrophobic side chain. In some embodiments, the side chain of X¹⁰ is C₁₋₆ aliphatic. In some embodiments, the side chain of X¹⁰ is C₁₋₆ alkyl. In some embodiments, X¹⁰ comprises a side chain comprising a polar group. In some embodiments, X¹⁰ is D-Met. In some embodiments, X¹⁰ is D-Nle. In some embodiments, X¹⁰ is D-Ile. In some embodiments, X¹⁰ is D-Val. In some embodiments, X¹⁰ is D-Leu. In some embodiments, X¹⁰ is D-Ala.

Various amino acid residues may be utilized for X¹². For example, in some embodiments, X¹² comprises a hydrophobic side chain. In some embodiments, X¹² comprises a side chain comprising a polar group. In some embodiments, X¹² comprises a side chain comprising an acidic group. In some embodiments, X¹² comprises a side chain comprising —COOH or a salt form thereof. In some embodiments, X¹² is D-Gln.

Various amino acid residues may be utilized for X¹³. For example, in some embodiments, X¹³ comprises a hydrophobic side chain. In some embodiments, X¹³ comprises a side chain comprising a polar group. In some embodiments, X¹³ comprises a side chain comprising an acidic group. In some embodiments, X¹³ comprises a side chain comprising —COOH or a salt form thereof. In some embodiments, X¹³ comprises a side chain comprising an aromatic group. In some embodiments, X¹³ comprises a side chain comprising a basic group. In some embodiments, X¹³ is D-Glu. In some embodiments, X¹³ is D-Asp. In some embodiments, X¹³ is D-Gln.

Various amino acid residues may be utilized for X¹⁴. For example, in some embodiments, X¹⁴ comprises a hydrophobic side chain. In some embodiments, X¹⁴ comprises a side chain comprising a polar group. In some embodiments, X¹⁴ comprises a side chain comprising an acidic group. In some embodiments, X¹⁴ comprises a side chain comprising —COOH or a salt form thereof. In some embodiments, X¹⁴ comprises a side chain comprising an aromatic group. In some embodiments, X¹⁴ comprises a side chain comprising a basic group. In some embodiments, X¹⁴ is D-Glu. In some embodiments, X¹⁴ is D-Asp. In some embodiments, X¹⁴ is D-Gln.

In some embodiments, an agent is or comprises H103. In some embodiments, X⁴³ (e.g., dW), X² (e.g., dY), X⁵ (e.g., dnL), X⁶ (e.g., dE), X⁹ (e.g., dnL), and/or X¹² (e.g., dQ) interact with MDM2. For example, in some embodiments, dW4, dY5, dnL8, dE9, dnL12, and/or dQ15 of H103 interact with MDM2. In some embodiments, dQ16 of H103 interacts with MDM2.

In some embodiments, an agent comprise a sequence selected from lvecnlaadmchfy, antciwaaiecsmy, watcmdaalnclqm, smcqwaahlceyw, laqcrwaawrcdfe and swqcvmaamdcvld, or a homolog thereof, wherein each amino acid residue is a D-amino acid residue. In some embodiments, a homolog comprises one or more or all amino acid residues interacting with MDM2. In some embodiments, a homolog comprises one or more amino acid residues that are, or have similar or comparable to, amino acid residues interacting with MDM2 and/or otherwise contributing to binding to MDM2. In some embodiments, a homolog binds to MDM2. Technologies for identifying amino acid residues interacting with or contributing to binding to MDM2, e.g., mutational studies, structural biology, etc., are widely available and can be utilized in accordance with the present disclosure.

In some embodiments, an agent is or comprises a peptide having the structure of:

R^{N}—[X]ₚ—[X⁰]ₚ₀X¹X²X³X⁴X⁵X⁶X⁷X⁸X⁹X¹⁰X¹¹X¹²X¹³X¹⁴[X¹⁵]ₚ₁₅[X¹⁶]ₚ₁₆[X¹⁷]ₚ₁₇—[X]_{p′}—R^{C},

or a salt thereof, wherein:
each of X, X¹, X², X³, X⁴, X⁵, X⁶, X⁷, X⁸, X⁹, X¹⁰, X¹¹, X¹², X¹³, X¹⁴, X¹⁵, X⁶ and X¹⁷ is independently an amino acid residue;
each p and p′ is independently 0-10;
each of p0, p15, p16 and p17 is independently 0 or 1;
R^{N} is independently a peptide, an amino protecting group or R′-L^{RN}-;
R^{C} is independently a peptide, a carboxyl protecting group, -L^{RC}-R′, —O-L^{RC}-R′ or —N(R′)-L R^{C}—R′;
each of L^{RN} and L^{RC} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, X¹X²X³X⁴X⁵X⁶X⁷X⁸X⁹X¹⁰X¹¹X¹²X¹³X¹⁴ is a sequence of cluster 31 as described herein. In some embodiments, X¹X²X³X⁴X⁵X⁶X⁷X⁸X⁹X¹⁰X¹¹X¹²X¹³X¹⁴ is a sequence of cluster 32 as described herein. In some embodiments, X¹X²X³X⁴X⁵X⁶X⁷X⁸X⁹X¹⁰X¹¹X¹²X¹³X¹⁴ is a sequence of cluster 33 as described herein. In some embodiments, X¹X²X³X⁴X⁵X⁶X⁷X⁸X⁹X¹⁰X¹¹X¹²X¹³X¹⁴ is a sequence of cluster 41 as described herein. In some embodiments, X¹X²X³X⁴X⁵X⁶X⁷X⁸X⁹X¹⁰X¹¹X¹²X¹³X¹⁴ is a sequence of cluster 42 as described herein. In some embodiments, X¹X²X³X⁴X⁵X⁶X⁷X⁸X⁹X¹⁰X¹¹X¹²X¹³X¹⁴ is a sequence of cluster 43 as described herein.

In some embodiments, p is 0. In some embodiments, p is 1, 2, 3, 4, 5, 6, 7, 8, 9, or 10. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, p0 is 0. In some embodiments, p0 is 1. In some embodiments, p15 is 0. In some embodiments, p15 is 1. In some embodiments, p16 is 0. In some embodiments, p16 is 1. In some embodiments, p17 is 0. is p17 is 1.

In some embodiments, p′ is 0. In some embodiments, p′ is 1, 2, 3, 4, 5, 6, 7, 8, 9, or 10. In some embodiments, p′ is 1. In some embodiments, p′ is 2. In some embodiments, p′ is 3. In some embodiments, p′ is 4. In some embodiments, p′ is 5. In some embodiments, p′ is 6. In some embodiments, p′ is 7. In some embodiments, p′ is 8. In some embodiments, p′ is 9. In some embodiments, p′ is 10.

In some embodiments, X¹ is X³¹. In some embodiments, X¹ is X³². In some embodiments, X¹ is X³³. In some embodiments, X¹ is X⁴¹. In some embodiments, X¹ is X⁴². In some embodiments, X¹ is X⁴³.

In some embodiments, R^{N} is an N-terminus capping group. In some embodiments, R^{N} is —C(O)R, wherein R is as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R^{N} is Ac. In some embodiments, R^{N} is a group suitable for stapling, or is stapled. In some embodiments, R^{N} is 4pentenyl. In some embodiments, R^{N} is 5hexenyl. In some embodiments, R^{N} is BzAm2OAllyl. In some embodiments, R^{N} is Ac, NPyroR3, 5hexenyl, 4pentenyl, Bua, C3a, Cpc, Cbc, CypCO, Bnc, CF3CO, 2PyCypCO, 4THPCO, Isobutyryl, Ts, 15PyraPy, 2PyBu, 4PymCO, 4PyPrpc, 3IAPAc, 4MePipzPrpC, MePipAc, MeImid4SO2, BzAm2OAllyl, Hex, 2PyzCO, 3Phc3, MeOPr, lithocholate, 2FPhc, PhC, MeSO2, Isovaleryl, EtHNCO, TzPyr, 8IAP, 3PydCO, 2PymCO, 5PymCO, 1Imidac, 2F2PyAc, 2IAPAc, 124TriPr, 6QuiAc, 3PyAc, 123TriAc, 1PyrazoleAc, 3PyPrpc, 5PymAc, 1PydoneAc, 124TriAc, Me2NAc, 8QuiSO2, mPEG4, mPEG8, mPEG16 or mPEG24.

In some embodiments, R^{C} is a C-terminus capping group. In some embodiments, R^{C} comprises a PEG moiety. In some embodiments, R^{C} is —O-L^{RC}-R′ wherein each of L^{RC} and R′ is independently as described herein. In some embodiments, R^{C} is —OR′, wherein R′ is optionally substituted C₁-C₁₀ aliphatic. In some embodiments, R^{C} is —OR′, wherein R′ is optionally substituted C₁-C₁₀ alkyl. In some embodiments, R^{C} is —OR′, wherein R′ is optionally substituted C₁₋₃₀ heteroaliphatic having 1-10 heteroatoms. In some embodiments, R^{C} is -L^{RC}-R′, wherein one or more methylene unit of L^{RC} are replaced with —O— and R′ is as described herein. In some embodiments, R^{C} is —O-L^{RC}-R′, wherein L^{RC} is optionally substituted C₁₋₆ alkylene and R′ is as described herein. In some embodiments, R^{C} is —O-L^{RC}-R′, wherein L^{RC} is optionally substituted C₁₋₆ alkylene and R′ is an optionally substituted group selected from C₆₋₃₀ aryl and 5-30 membered heteroaryl having 1-10 heteroatoms. In some embodiments, R^{C} is —OR′ wherein R′ is optionally substituted C₆₋₃₀ arylaliphatic. In some embodiments, R^{C} is —N(R′)₂ wherein each R′ is independently as described herein. In some embodiments, R^{C} is —NHR′ wherein R′ is as described herein. In some embodiments, R^{C} is —N(R)₂ wherein each R is independently as described herein. In some embodiments, R^{C} is —NHR wherein R is as described herein. In some embodiments, R is —H. In some embodiments, R is optionally substituted C₁₋₆ aliphatic. In some embodiments, R is optionally substituted C₁₋₆ alkyl. In some embodiments, R is methyl. In some embodiments, R is ethyl. In some embodiments, R^{C} is —NH₂. In some embodiments, R^{C} is —NHEt.

In some embodiments, R^{C} is —NHC(CH₃)CH₂OH. In some embodiments, R^{C} is —(S)—NHC(CH₃)CH₂OH. In some embodiments, R^{C} is —(R)—NHC(CH₃)CH₂OH. In some embodiments, R^{C} is

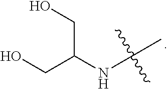 [SMILES: *NC(CO)CO]

In some embodiments, R^{C} is

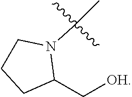 [SMILES: *N1CCCC1CO]

In some embodiments, R^{C} is

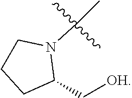 [SMILES: *N1CCC[C@H]1CO]

In some embodiments, R^{C} is

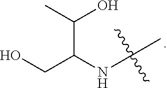 [SMILES: *NC(CO)C(C)O]

In some embodiments, R^{C} is

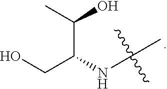 [SMILES: *N[C@H](CO)[C@@H](C)O]

In some embodiments, R^{C} is -Alaol, wherein the amino group of -Alaol is bonded to the last —C(O)— of the peptide backbone (R^{C} is

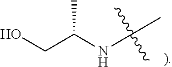 [SMILES: *N[C@@H](C)CO]

In some embodiments, R^{C} is -dAlaol, wherein the amino group of -dAlaol is bonded to the last —C(O)— of the peptide backbone (R^{C} is

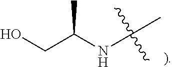 [SMILES: *N[C@H](C)CO]

In some embodiments, R^{C} is -Prool, wherein the amino group of -Prool is bonded to the last —C(O)— of the peptide backbone (R^{C} is

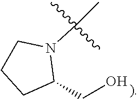 [SMILES: *N1CCC[C@H]1CO]

In some embodiments, R^{C} is -Throl, wherein the amino group of -Throl is bonded to the last —C(O)— of the peptide backbone (R^{C} is

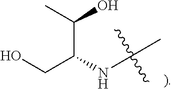 [SMILES: *N[C@H](CO)[C@@H](C)O]

In some embodiments, R^{C} is -Serol, wherein the amino group of -Serol is bonded to the last —C(O)— of the peptide backbone (R^{C} is

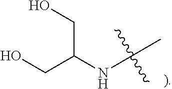 [SMILES: *NC(CO)CO]

In some embodiments, R^{C} is —OH.

### Amino Acids

As appreciated by those skilled in the art, various amino acids may be utilized in accordance with the present disclosure. For example, both naturally occurring and non-naturally occurring amino acids can be utilized in accordance with the present disclosure. In some embodiments, an amino acid is a compound comprising an amino group that can form an amide group with a carboxyl group and a carboxyl group. In some embodiments, an amino acid is an alpha amino acid. In some embodiments, an amino acid is a beta-amino acid. In some embodiments, an amino acid is a D-amino acid. In some embodiments, an amino acid is a L-amino acid. In some embodiments, an amino acid is an naturally encoded amino acid, e.g., in mammalian cells.

In some embodiments, an amino acid is a compound having the structure of formula A-I:

N(R^{a1})₂-L^{a1}-C(R^{a2})(R^{a3})-L^{a2}-COOH, A-I

or a salt thereof, wherein:
each of R^{a1}, R^{a2}, R^{a3} is independently -L^{a}-R′;
each of L^{a}, L^{a1} and L^{a2} is independently L;
each L is independently a covalent bond, or an optionally substituted, bivalent C₁-C₂₅ aliphatic or heteroaliphatic group having 1-10 heteroatoms, wherein one or more methylene units of the group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent, 3-30 membered, monocyclic, bicyclic or polycyclic ring having 0-10 heteroatoms;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms, C₆₋₃₀ aryl, C₆₋₃₀ arylaliphatic, C₆₋₃₀ arylheteroaliphatic having 1-10 heteroatoms, 5-30 membered heteroaryl having 1-10 heteroatoms, and 3-30 membered heterocyclyl having 1-10 heteroatoms, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, a compound having the structure of formula A-I or a salt thereof has the structure of NH(R^{a1})-L^{a1}-C(R^{a2})(R^{a3})-L^{a2}-COOH or a salt thereof.

In some embodiments, a ring moiety of, e.g., -Cy-, R (including those formed by R groups taken together), etc. is monocyclic. In some embodiments, a ring moiety is bicyclic or polycyclic. In some embodiments, a monocyclic ring is an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms. In some embodiments, each monocyclic ring unit of a bicyclic or polycyclic ring moiety is independently an optionally substituted 3-10 (3, 4, 5, 6, 7, 8, 9, or 10, 3-8, 3-7, 4-7, 4-6, 5-6, etc.) membered, saturated, partially unsaturated or aromatic ring having 0-5 heteroatoms.

In some embodiments, each heteroatom is independently selected from oxygen, nitrogen, sulfur, phosphorus and silicon. In some embodiments, each heteroatom is independently selected from oxygen, nitrogen, and sulfur.

In some embodiments, L^{a1} is a covalent bond. In some embodiments, a compound of formula A-1 is of the structure NH(R^{a1})—C(R^{a2})(R^{a3})-L^{a2}-COOH.

In some embodiments, L^{a2} is a covalent bond. In some embodiments, a compound of formula A-1 is of the structure NH(R^{a1})—C(R^{a2})(R^{a3})-L^{a2}-COOH.

In some embodiments, L^{a1} is a covalent bond and L^{a2} is a covalent bond. In some embodiments, a compound of formula A-1 is of the structure NH(R^{a1})—C(R^{a2})(R^{a3})—COOH.

In some embodiments, an amino acid is suitable for stapling. In some embodiments, an amino acid comprises a terminal olefin. Certain such amino acids are exemplified herein (e.g., those described in or utilized in peptides of various Tables).

In some embodiments, an agent comprises a detectable moiety, which can either be detected directly or indirectly. For example, in some embodiments, a detectable moiety is or comprises a fluorescent group. In some embodiments, a detectable moiety is or comprises a biotin moiety. In some embodiments, a detectable moiety is connected to the rest of an agent at an amino acid residue, e.g., through a side chain, optionally through a linker (e.g., L as described herein). In some embodiments, a detectable moiety is —N₃, which may be detected after a click chemistry reaction with a labeled agent comprising an alkyne.

In some embodiments, the present disclosure provides various compounds, which among other things may be utilized as amino acids for a number of applications, e.g., for preparation of peptides or other useful compounds.

In some embodiments, a compound (e.g., an amino acid or a protected and/or activated form thereof) or a salt thereof comprises 1) a first group which is an optionally protected amino group, 2) a second group which is an optionally protected and/or activated carboxyl group, and 3) a side chain (typically bonded to an atom between the first and second groups (“a side chain attachment atom”)) which comprises an optionally protected and/or activated carboxyl group and a) an optionally substituted ring (which ring is typically between the optionally protected and/or activated carboxyl group of the side chain and a side chain attachment atom) or b) an amino group (which amino group is typically between the optionally protected and/or activated carboxyl group of the side chain and a side chain attachment atom). In some embodiments, a provided compound is an optionally protected and/or activated amino acid or a salt thereof, wherein the side chain of the amino acid comprises an optionally protected and/or activated carboxyl group, and an optionally substituted ring or an amino group, wherein the optionally substituted ring or an amino group is between the optionally protected and/or activated carboxyl group and a backbone atom to which a side chain is attached (e.g., an atom between an amino and carboxyl group, both of which can be optionally and independently protected and/or activated (e.g., an alpha carbon atom in an amino acid)).

Certain useful amino acids are described in, e.g., WO 2019/051327, WO 2022/020652, WO/2022/261257, etc., the amino acids of each of which are incorporated herein by reference.

### Characterization

In some embodiments, agents, e.g., peptides, are characterized with respect to, for example, one or more characteristics such as binding characteristics—e.g., with respect to a particular target of interest (e.g., MDM2 or CHIP, or a portion thereof), stability characteristics, for example in solution or in dried form, cell permeability characteristics, solubility, lipophilicity, etc.

In some embodiments, a binding characteristic may be or comprise specificity, affinity, on-rate, off-rate, etc, optionally under (or over a range of) specified conditions such as, for example, concentration, temperature, pH, cell type, presence or level of a particular competitor, etc.

As will be appreciated by those skilled in the art, assessments of characteristics as described herein may involve comparison with an appropriate reference (e.g., a positive or negative control) which may, in some embodiments, be a contemporaneous reference or, in some embodiments, a historical reference.

In some embodiments, desirable characteristics may be, for example: binding to a desired target (e.g., a dissociation constant (K_{D}) of at least less than about 1 μM, and preferably a K_{D} of less than about 50 nM); cell penetration (e.g., as measured by fluorescence-based assays or mass spectrometry of cellular fractions, etc.); solubility (e.g., soluble at less than about 1000 μM agent, or soluble at less than about 500 μM agent, or soluble at less than about 100 μM agent, or less than about 50 μM, or less than about 35 μM); activity (e.g., modulating one or more functions of a target, which may be assessed in a cellular reporter assay (e.g., with an IC50 of less than a concentration, e.g., less than about 1 μM, less than about 500 nM, less than about 50 nM, less than about 10 nM, etc.), an animal model (e.g., various animal models for conditions, disorders or diseases) and/or a subject; stability, which may be assessed using a number of assays (e.g., in a rat pharmacokinetic study (e.g., administered via oral, iv, ip, etc.) with a terminal half-life of greater than a suitable time, e.g., 1 hour); low toxicity, which might be assessed by a number of assays (e.g., a standard ADME/toxicity assays); and/or low levels of cytotoxicity (e.g., low levels of lactate dehydrogenase (LDH) released from cells when treated at a suitable concentration, e.g., about 10 μM of a peptide). In some embodiments, an agent of the invention comprises an affinity of less than about 10 nM, for example, an IC50 of 7 nM).

In some embodiments, provided agents can bind to targets with an EC 50 of no more than about 2000 nM. In some embodiments, an EC50 is no more than about 1500 nM. In some embodiments, an EC50 is no more than about 1000 nM. In some embodiments, an EC50 is no more than about 500 nM. In some embodiments, an EC50 is no more than about 300 nM. In some embodiments, an EC50 is no more than about 200 nM. In some embodiments, an EC50 is no more than about 100 nM. In some embodiments, an EC50 is no more than about 75 nM. In some embodiments, an EC50 is no more than about 50 nM. In some embodiments, an EC50 is no more than about 25 nM. In some embodiments, an EC50 is no more than about 10 nM. In some embodiments, an EC50 is no more than about 5 nM. In some embodiments, an EC50 is measured by fluorescence polarization as described in the Examples.

In some embodiments, the present disclosure provides agents, e.g., stapled peptides, with suitable solubility for various purposes. In some embodiments, solubility of provided agents, e.g., in PBS, is about or at least about 5-100 μM (e.g., about or at least about 5, 10, 15, 20, 25, 30, 40, 50, 60, 70, 80, 90, or 100 μM). In some embodiments, solubility is about or at least about 25 μM. In some embodiments, solubility is about or at least about 30 μM. In some embodiments, solubility is about or at least about 40 μM. In some embodiments, solubility is about or at least about 50 μM. In some embodiments, provided agents, e.g., stapled peptides, are protein bound in serum; in some embodiments, they are at least about 85%, 90%, or 95% protein bound in serum. In some embodiments, provided agents are over 95% protein bound in serum.

In some embodiments, provided agents can traverse a cell membrane of an animal cell. In some embodiments, provided agents can traverse a cell membrane of a human cell.

Among other things, provided agents can bind to motifs, residues, or polypeptides. In some embodiments, provided agents bind to MDM2. In some embodiments, provided agents bind to CHIP. In some embodiments, a dissociation constant (K_{D}) is about 1 nM to about 1 μM. In some embodiments, a K_{D} is no more than about 1 μM. In some embodiments, a K_{D} is no more than about 500 nM. In some embodiments, a K_{D} is no more than about 250 nM. In some embodiments, a K_{D} is no more than about 100 nM. In some embodiments, a K_{D} is no more than about 50 nM. In some embodiments, a K_{D} is no more than about 25 nM. In some embodiments, a K_{D} is no more than about 10 nM. In some embodiments, a K_{D} is no more than about 5 nM. In some embodiments, a K_{D} is no more than about 1 nM. As appreciated by those skilled in the art, various technologies are available and can be utilized to measure K_{D} in accordance with the present disclosure. In some embodiments, K_{D} is measured by Surface Plasmon Resonance (SPR) as illustrated herein.

In some embodiments, provided agents binds to a polypeptide whose sequence is or comprising SEQ ID NO: 1, or a fragment thereof: (SEQ ID NO: 1):

(SEQ ID NO: 1)
MCNTNMSVPTDGAVTTSQIPASEQETLVRPKPLLLKLLKSVGAQKDTYT
MKEVLFYLGQYIMTKRLYDEKQQHIVYCSNDLLGDLFGVPSFSVKEHRK
IYTMIYRNLVVVNQQESSDSGTSVSENRCHLEGGSDQKDLVQELQEEKP
SSSHLVSRPSTSSRRRAISETEENSDELSGERQRKRHKSDSISLSFDES
LALCVIREICCERSSSSESTGTPSNPDLDAGVSEHSGDWLDQDSVSDQF
SVEFEVESLDSEDYSLSEEGQELSDEDDEVYQVTVYQAGESDTDSFEED
PEISLADYWKCTSCNEMNPPLPSHCNRCWALRENWLPEDKGKDKGEISE
KAKLENSTQAEEGFDVPDCKKTIVNDSRESCVEENDDKITQASQSQESE
DYSQPSTSSSIIYSSQEDVKEFEREETQDKEESVESSLPLNAIEPCVIC
QGRPKNGCIVHGKTGHLMACFTCAKKLKKRNKPCPVCRQPIQMIVLTYF
P.

In some embodiments, a fragment is or comprises p53 binding domain or characteristic residues thereof.

In some embodiments, provided agents binds to a polypeptide whose sequence is or comprising SEQ ID NO: 2, or a fragment thereof: (SEQ ID NO: 2):

(SEQ ID NO: 2)
MKGKEEKEGGARLGAGGGSPEKSPSAQELKEQGNRLFVGRKYPEAAACY
GRAITRNPLVAVYYTNRALCYLKMQQHEQALADCRRALELDGQSVKAHF
FLGQCQLEMESYDEAIANLQRAYSLAKEQRLNFGDDIPSALRIAKKKRW
NSIEERRIHQESELHSYLSRLIAAERERELEECQRNHEGDEDDSHVRAQ
QACIEAKHDKYMADMDELFSQVDEKRKKRDIPDYLCGKISFELMREPCI
TPSGITYDRKDIEEHLQRVGHFDPVTRSPLTQEQLIPNLAMKEVIDAFI
SENGWVEDY.

In some embodiments, a fragment is or comprises TPR domain or characteristic residues thereof.

In some embodiments, an agent, e.g., a peptide, binds to MDM2 and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 1 at the indicated positions:

In some embodiments, an agent, e.g., a peptide, binds to CHIP and interacts with one or more residues that are or correspond to at least two, or at least three, or at least four, or at least five, or at least six, or at least seven, or at least eight or at least nine, or at least ten, or at least eleven, or at least twelve, or at least thirteen, or at least fourteen, or at least fifteen, or at least sixteen, or at least seventeen, or at least eighteen, or at least nineteen, or at least twenty of the following amino acid residues in SEQ ID NO: 2 at the indicated positions:

Various technologies can be utilized for characterizing and/or assessing provided technologies (e.g., agents (e.g., various peptides), compositions, methods, etc.) in accordance with the present disclosure. As described herein, in some embodiments, a useful technology is or comprises fluorescence polarization. In some embodiments, a useful technology assesses Log P or Log D. In some embodiments, a useful technology is or comprises a CHI Log D assay. In some embodiments, a useful technology assesses solubility. In some embodiments, a useful technology is or comprises NanoBRET. In some embodiments, a useful technology is or comprises a reporter assay (e.g., DLD1 reporter assay). In some embodiments, a useful technology is or comprises alphascreen. Certain useful protocols are described in the Examples. Those skilled in the art appreciate that suitable adjustments may be made to such protocols, e.g., according to specific conditions, agents, purposes, etc.

### Production

Various technologies are known in the art for producing stapled peptides of may be utilized in accordance with the present disclosure. Those skilled in the art, reading the present disclosure, will well appreciate which such technologies are applicable in which aspects of the present disclosure. Certain technologies are described U.S. Ser. No. 11/198,713, US 20210179665, WO 2021119537, WO 2021188659, WO 2022020651, WO 2022020652, or WO/2022/261257, the peptide production technologies of each of which are incorporated herein by reference.

In some embodiments, as described herein, certain stapled peptides, and in particular cysteine stapled peptides, may be provided in and/or produced by a biological system and reacting with a provided reagent, e.g., one having the structure of R^{x}-L^{s2}-R^{x} or a salt thereof.

In some embodiments, peptides are prepared on solid phase on a synthesizer using, typically, Fmoc chemistry.

In some embodiments, staples are formed by olefin metathesis. In some embodiments, a product double bond of metathesis is reduced/hydrogenated. In some embodiments, CO₂ are extruded from a carbamate moiety of a staple. In some embodiments, provided stapled peptides are further modified, and/or conjugated to other entities. Conditions and/or reagents of these reactions are widely known in the art and can be performed in accordance with the present disclosure to provide stapled peptides.

Properties and/or activities of provided stapled peptides can be readily assessed in accordance with the present disclosure, for example, through use of one or more methods described in the examples.

In some embodiments, technologies for preparing and/or assessing provided stapled peptides include those described in U.S. Pat. No. 9,617,309, US 2015-0225471, US 2016-0024153, US 2016-0215036, US2016-0244494, WO 2017/062518, etc.

In some embodiments, a provided agent, e.g., a provided peptide, has a purity of 60%-100%. In some embodiments, a provided agent has a purity of at least 60%, 65%, 70%, 75%, 80%, 85%, 90%, 91%, 92%, 93%, 94%, 95%, 96%, 97%, 98%, or 99%. In some embodiments, a purity is at least 60%. In some embodiments, a purity is at least 70%. In some embodiments, a purity is at least 80%. In some embodiments, a purity is at least 85%. In some embodiments, a purity is at least 90%. In some embodiments, a purity is at least 91%. In some embodiments, a purity is at least 92%. In some embodiments, a purity is at least 93%. In some embodiments, a purity is at least 94%. In some embodiments, a purity is at least 95%. In some embodiments, a purity is at least 96%. In some embodiments, a purity is at least 97%. In some embodiments, a purity is at least 98%. In some embodiments, a purity is at least 99%. In some embodiments, a purity is at least 99.5%.

In some embodiments, provided methods provide high yields. In some embodiments, a yield is 50%-100%. In some embodiments, a yield is at least 60%, 65%, 70%, 75%, 80%, 85%, 90%, 91%, 92%, 93%, 94%, 95%, 96%, 97%, 98% or 99%. In some embodiments, a yield is at least 60%. In some embodiments, a yield is at least 65%. In some embodiments, a yield is at least 70%. In some embodiments, a yield is at least 75%. In some embodiments, a yield is at least 80%. In some embodiments, a yield is at least 85%. In some embodiments, a yield is at least 90%. In some embodiments, a yield is at least 91%. In some embodiments, a yield is at least 92%. In some embodiments, a yield is at least 93%. In some embodiments, a yield is at least 94%. In some embodiments, a yield is at least 95%. In some embodiments, a yield is at least 96%. In some embodiments, a yield is at least 97%. In some embodiments, a yield is at least 98%. In some embodiments, a yield is at least 99%.

In some embodiments, a provided method delivers high E/Z selectivity for olefin. In some embodiments, provided selectivity favors the E isomer. In some embodiments, provided selectivity favors the Z isomer. In some embodiments, a E:Z ratio is at least 1:1, 1.5:1, 2:1, 3:1, 4:1, 5:1, 6:1, 7:1, 8:1, 9:1, 10:1, 20:1, 30:1, 40:1, 50:1, or 100:1. In some embodiments, a Z:E ratio is at least 1:1, 1.5:1, 2:1, 3:1, 4:1, 5:1, 6:1, 7:1, 8:1, 9:1, 10:1, 20:1, 30:1, 40:1, 50:1, 80:1, 90:1, 95:1, 99:1, or 100:1. In some embodiments, a ratio is at least 1:1. In some embodiments, a ratio is at least 1.5:1. In some embodiments, a ratio is at least 2:1. In some embodiments, a ratio is at least 3:1. In some embodiments, a ratio is at least 4:1. In some embodiments, a ratio is at least 5:1. In some embodiments, a ratio is at least 6:1. In some embodiments, a ratio is at least 7:1. In some embodiments, a ratio is at least 8:1. In some embodiments, a ratio is at least 9:1. In some embodiments, a ratio is at least 10:1. In some embodiments, a ratio is at least 20:1. In some embodiments, a ratio is at least 30:1. In some embodiments, a ratio is at least 40:1. In some embodiments, a ratio is at least 50:1. In some embodiments, a ratio is at least 80:1. In some embodiments, a ratio is at least 90:1. In some embodiments, a ratio is at least 95:1. In some embodiments, a ratio is at least 99:1. In some embodiments, a ratio is at least 100:1.

In some embodiments, a provide method comprises a period of time at a temperature higher than room temperature. In some embodiments, a temperature is about 25-200° C. In some embodiments, a temperature is about 25° C. In some embodiments, a temperature is about 30° C. In some embodiments, a temperature is about 35° C. In some embodiments, a temperature is about 40° C. In some embodiments, a temperature is about 45° C. In some embodiments, a temperature is about 50° C. In some embodiments, a temperature is about 55° C. In some embodiments, a temperature is about 60° C. In some embodiments, a temperature is about 65° C. In some embodiments, a temperature is about 70° C. In some embodiments, a temperature is about 75° C. In some embodiments, a temperature is about 80° C. In some embodiments, a temperature is about 85° C. In some embodiments, a temperature is about 90° C. In some embodiments, a temperature is about 95° C. In some embodiments, a temperature is about 100° C. In some embodiments, a temperature is about 150° C. In some embodiments, a temperature is higher than about 150° C.

### Compositions

Among other things, the present disclosure provides compositions that comprise or otherwise relate to provided agents, e.g., small molecule agents, peptide agents (e.g., stapled peptides), as described herein.

In some embodiments, provided compositions are or comprise an assay system for characterizing (and optionally including) a stapled peptide as described herein.

In some embodiments, provided compositions are pharmaceutical compositions e.g., that comprise or deliver one or more provided agents.

In some embodiments, an agent is a peptide. In some embodiments, an agent is a stapled peptide.

In some embodiments, a pharmaceutical composition comprises a provided agent and a pharmaceutically acceptable carrier.

In some embodiments, a peptide composition may include or deliver a particular form (e.g., a particular optical isomer, diastereomer, salt form, covalent conjugate form [e.g., covalently attached to a carrier moiety], etc., or combination thereof) of an agent as described herein). In some embodiments, an agent included or delivered by a pharmaceutical composition is described herein is not covalently linked to a carrier moiety.

In some embodiments, multiple stereoisomers exist for an agent that contains chiral centers and/or double bonds. In some embodiments, level of a particular agent in a composition is enriched relative to one or more or all of its stereoisomers. For example, in some embodiments, a particularly configuration of a double bond (E Z) is enrich. In some embodiments, for each double bond a configuration is independently enriched. In some embodiments, for a chiral element, e.g., a chiral center, one configuration is enriched. In some embodiments, for a chiral center bonded to two staples, one configuration is enriched. In some embodiments, for each chiral element a configuration is independently enriched. In some embodiments, for one or more or all stereochemical element (e.g., double bonds, chiral element, etc.), one configuration is independently enriched. In some embodiments, for each double bond in each staple, one configuration is independently enriched. In some embodiments, for each double bond in each staple, one configuration is independently enriched, and for a chiral center bonded to two staples, one configuration is enriched. In some embodiments, enrichment for each double bond is independently E or Z. In some embodiments, enrichment for each chiral element is independently R or S. In some embodiments, enrichment for each stereochemical element, e.g., double bond, chiral center, etc., is about or at least about a certain level, e.g., 60%, 65%, 70%, 75%, 80%, 85%, 90%, 91%, 92%, 93%, 94%, 95%, 96%, 97%, 98%, or 99% (percentage of an agent). In some embodiments, about or at least about a certain level, e.g., 60%, 65%, 70%, 75%, 80%, 85%, 90%, 91%, 92%, 93%, 94%, 95%, 96%, 97%, 98%, or 99% of all molecules in a composition that share the constitution of an agent or a salt thereof are the agent or a salt thereof. In some embodiments, a level is about or at least about 60%. In some embodiments, it is about or at least about 65%. In some embodiments, it is about or at least about 70%. In some embodiments, it is about or at least about 75%. In some embodiments, it is about or at least about 80%. In some embodiments, it is about or at least about 85%. In some embodiments, it is about or at least about 90%. In some embodiments, it is about or at least about 95%. In some embodiments, it is about or at least about 96%. In some embodiments, it is about or at least about 97%. In some embodiments, it is about or at least about 98%. In some embodiments, it is about or at least about 99%.

In some embodiments, a provided therapeutic composition may comprise one or more additional therapeutic agents and/or one or more stabilizing agents and/or one or more agents that alters (e.g., extends or limits to a particular tissue, location or site) rate or extent of delivery over time.

In some embodiments, a composition is a pharmaceutical composition which comprises or delivers a provided agent (e.g., a stapled peptide) or a pharmaceutically acceptable salt thereof and a pharmaceutically acceptable excipient. In some embodiments, a composition comprises one and only stereoisomer of an agent (e.g., a stapled peptide) and/or one or more salts thereof. In some embodiments, a composition comprises two or more stereoisomers of an agent (e.g., a stapled peptide) and/or one or more salts thereof. In some embodiments, the two or more stereoisomers of an agent (e.g., a stapled peptide) or salts thereof elute as a single peak (e.g., UV and/or MS detection) in a chromatography, e.g., HPLC.

### Uses and Applications

Provided agents and compositions can be utilized for various purposes. For example, certain compounds may be utilized as amino acids, either directly or for preparation of other compounds such as peptides. Certain agents, e.g., peptides, may be utilized to prepare stapled peptides. Certain agents that are or comprise peptides, particularly stapled peptides, and compositions thereof, are biologically active and can be utilized for various purposes, e.g., as therapeutics toward various conditions, disorders or diseases, as tools for modulating biological functions, etc.

In some embodiments, the present disclosure provides agents and compositions thereof for modulating target polypeptide functions. In some embodiments, provided agents can bind a target polypeptide. In some embodiments, a target polypeptide is or comprises MDM2 or a fragment thereof. In some embodiments, a target polypeptide is or comprises CHIP or a fragment thereof.

In some embodiments, the present disclosure provides a method for modulating a target polypeptide function, comprising contacting the target polypeptide with a provided agent. In some embodiments, the present disclosure provides a method for modulating a target polypeptide function in a system, comprising administering or delivering to the system an effective amount of a provided agent, wherein the system comprises the target polypeptide. In some embodiments, the present disclosure provides a method for modulating a target polypeptide function in a system, comprising administering or delivering to the system an effective amount of a provided agent, wherein the system expresses the target polypeptide. In some embodiments, a system is in vitro. In some embodiments, a system is in vivo. In some embodiments, a system is or comprises a cell. In some embodiments, a system is or comprises a population of cells. In some embodiments, a cell is a diseased cell. In some embodiments, a cell is a cancer cell. In some embodiments, a system is or comprises a tissue. In some embodiments, a system is or comprises an organ. In some embodiments, a system is or comprises a subject.

In some embodiments, the present disclosure provides a method for preventing or treating a condition, disorder or disease associated with a target polypeptide, comprising administering or delivering to a subject susceptible thereto or suffering therefrom an effective amount of a provided agent, e.g., a stapled peptide agent. In some embodiments, a condition, disorder or disease is cancer.

In some embodiments, comparison is made to a reference. For example, reduction, increase, enrichment (negative or positive), changes, etc., are typically made to a suitable reference. In some embodiments, reduction, increase, enrichment (negative or positive), changes, etc., are to a reference assessment, in some embodiments, of a reference sample. In some embodiments, a reference assessment is or comprises assessment conducted prior to an administration or delivery of an agent. In some embodiments, a reference sample is collected prior to an administration or delivery of an agent. In some embodiments, a reference assessment is or comprises assessment conducted during an administration or delivery of an agent. In some embodiments, a reference sample is collected during an administration or delivery of an agent. In some embodiments, a reference assessment is or comprises assessment conducted after an administration or delivery of an agent. In some embodiments, a reference sample is collected after an administration or delivery of an agent. In some embodiments, a reference assessment is or comprises assessment conducted after an earlier administration or delivery of an agent. In some embodiments, a reference sample is collected after earlier an administration or delivery of an agent.

In some embodiments, a sample is an aliquot of material obtained or derived from a source of interest as described herein. In some embodiments, a source of interest is a biological or environmental source. In some embodiments, a source of interest may be or comprise a cell or an organism, such as a microbe, a plant, or an animal (e.g., a human). In some embodiments, a source of interest is or comprises biological tissue or fluid. In some embodiments, a biological tissue or fluid may be or comprise amniotic fluid, aqueous humor, ascites, bile, bone marrow, blood, breast milk, cerebrospinal fluid, cerumen, chyle, chime, ejaculate, endolymph, exudate, feces, gastric acid, gastric juice, lymph, mucus, pericardial fluid, perilymph, peritoneal fluid, pleural fluid, pus, rheum, saliva, sebum, semen, serum, smegma, sputum, synovial fluid, sweat, tears, urine, vaginal secreations, vitreous humour, vomit, and/or combinations or component(s) thereof. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological fluid may be or comprise a plant exudate. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., brocheoalvealar, ductal, nasal, ocular, oral, uterine, vaginal, or other washing or lavage). In some embodiments, a biological sample is or comprises cells obtained from an individual. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to one or more techniques such as amplification or reverse transcription of nucleic acid, isolation and/or purification of certain components, etc. In some embodiments, a sample comprise cancer cells. In some embodiments, a sample is obtained from a tumor. In some embodiments, a sample is obtained from a tumor in a patient.

In some embodiments, levels of one or more transcripts and/or products thereof may be assessed. In some embodiments, assessment is performed after one or more doses of agents, e.g., stapled peptides are administered or delivered to a subject. In some embodiments, if profiles, e.g., reduction, increase, etc., of one or more transcripts and/or products thereof matches those described herein, administration or delivery to a subject may continue. In some embodiments, if profiles, e.g., reduction, increase, etc., of one or more transcripts and/or products thereof matches those described herein, administration or delivery to a subject may be stopped and/or continued according to different dose levels and/or regimens.

Various technologies can be utilized in accordance with the present disclosure to formulate, distribute, administer or deliver provided technologies such as agents, peptides, compounds, compositions, etc. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g., intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. In some embodiments, provided technologies are administered intravenously.

Among other things, the present disclosure provides various structural moieties including designed amino acid residues that can be utilized to optimize various properties and activities, stability, delivery, pharmacodynamics, pharmacokinetics, etc. to provide various dosage forms, dosage regimen, therapeutic windows, etc. In some embodiments, provided agents and compositions thereof may be utilized with improved dosage regimen and/or unit doses. In some embodiments, administration of provided agents are adjusted based on conditions, disorders or diseases and/or subpopulations. In some embodiments, administration and/or dosage regimen of provided technologies are adjusted according to certain biomarkers and genomic alterations.

Provided agents may deliver biological effects, e.g., therapeutic effects, via various mechanisms. In some embodiments, efficacy may be driven by AUC. In some embodiments, efficacy may be driven by Cmax.

In some embodiments, a provided agent is utilized in combination with another therapy. In some embodiments, a provided agent is utilized in combination with another therapeutic agent. In some embodiments, another therapy or therapeutic agent is administered prior to an administration or delivery of a provided agent. In some embodiments, another therapy or therapeutic agent is administered at about the same time as an administration or delivery of a provided agent. In some embodiments, a provided agent and another agent is in the same pharmaceutical composition. In some embodiments, another therapy or therapeutic agent is administered subsequently to an administration or delivery of a provided agent. In some embodiments, a subject is exposed to both a provided agent and another therapeutic agent. In some embodiments, both a provided agent and another agent can be detected in a subject. In some embodiments, a provided agent is administered before another agent is cleared out by a subject or vice versa. In some embodiments, a provided agent is administered within the half-life, or 2, 3, 4, 5 or 6 times of the half-life, of another agent or vice versa. In some embodiments, a subject is exposed to a therapeutic effect of a provided agent and a therapeutic effect of another therapeutic agent. In some embodiments, an agent may provide an effect after an agent is cleared out or metabolized by a subject. In some embodiments, a procedure, e.g., surgery, radiation, etc., may provide an effect after the procedure is completed.

In some embodiments, another therapy is a cancer therapy. In some embodiments, another therapy is or comprises surgery. In some embodiments, another therapy is or comprises radiation therapy. In some embodiments, another therapy is or comprises immunotherapy. In some embodiments, another therapeutic agent is or comprises a drug. In some embodiments, another therapeutic agent is or comprises a cancer drug. In some embodiments, another therapeutic agent is or comprises a chemotherapeutic agent. In some embodiments, another therapeutic agent is or comprises a hormone therapy agent. In some embodiments, another therapeutic agent is or comprises a kinase inhibitor. In some embodiments, another therapeutic agent is or comprises a checkpoint inhibitor (e.g., antibodies against PD-1, PD-L1, CTLA-4, etc.). In some embodiments, a provide agent can be administered with lower unit dose and/or total dose compared to being used alone. In some embodiments, another agent can be administered with lower unit dose and/or total dose compared to being used alone. In some embodiments, one or more side effects associated with administration of a provided agent and/or another therapy or therapeutic agent are reduced. In some embodiments, a combination therapy provides improved results, e.g., when compared to each agent utilized individually. In some embodiments, a combination therapy achieves one or more better results, e.g., when compared to each agent utilized individually.

In some embodiments, another agent is a checkpoint inhibitor, an EGFR inhibitor, a VEGF inhibitor, a VEGFR inhibitor, a kinase inhibitor, or an anti-cancer drug.

In some embodiments, an additional agent is a checkpoint inhibitor. In some embodiments, an additional agent is an immune oncology agent. In some embodiments, an additional agent is an antibody against a checkpoint molecules. In some embodiments, an additional agent is an antibody of PD1, PDL-1, CTLA4, A2AR, B7-H3, B7-H4, BTLA, IDO, KIR, LAG3, TIM-s, C10orf54, etc. In some embodiments, an antibody is an anti-PD1 antibody. In some embodiments, an antibody is an anti-PD-L1 antibody. In some embodiments, an antibody is an anti-CTLA4.

In some embodiments, another agent is an EGFR inhibitor, e.g., erlotinib, gefitinib, lapatinib, panitumumab, vandetanib, cetuximab, etc. In some embodiments, another agent is an VEGF and/or VEGFR inhibitor, e.g., pazopanib, bevacizumab, sorafenib, sunitinib, axitinib, ponatinib, regorafenib, vandetanib, cabozantinib, ramucirumab, lenvatinib, ziv-aflibercept, etc. In some embodiments, another agent is a kinase inhibitor. In some embodiments, another therapeutic agent is a chemotherapeutic agent. In some embodiments, another therapeutic agent is an anti-cancer drug, e.g., cyclophosphamide, methotrexate, 5-fluorouracil (5-FU), doxorubicin, mustine, vincristine, procarbazine, prednisolone, dacarbazine, bleomycin, etoposide, cisplatin, epirubicin, capecitabine, folinic acid, actinomycin, all-trans retinoic acid, azacitidine, azathioprine, bortezomib, carboplatin, chlorambucil, cytarabine, daunorubicin, docetaxel, doxifluridine, fluorouracil, gemcitabine, hydroxyurea, idarubicin, imatinib, irinotecan, mechlorethamine, mercaptopurine, mitoxantrone, paclitaxel, pemetrexed, teniposide, tioguanine, topotecan, valrubicin, vinblastine, vindesine, vinorelbine, oxaliplatin, etc.

### EXEMPLIFICATION

Those skilled in the art appreciate that various technologies are available for manufacturing and assessing provided agents including various peptides such as stapled peptides in accordance with the present disclosure, for example, many technologies for preparing small molecules and peptides can be utilized to prepare provided agents, and various assays are available for assessing properties and/or activities of provided agents. In some embodiments, certain useful technologies are described in WO 2019/051327, WO 2022/020652, WO/2022/261257, etc. In some embodiments, certain useful technologies are described in Li, et al., Proc Natl Acad Sci USA 2022 Dec. 27; 119(52):e2210435119. doi: 10.1073/pnas.2210435119. In some embodiments, Fmoc-based chemistry is utilized for peptide synthesis using various commercially available reagents such as Fmoc protected L- or D-amino acids. Described below are certain such useful technologies as examples.

### Example 1. The Present Disclosure Provides Agents that Bind MDM2

Among other things, the present disclosure provides MDM2-binding agents and compositions thereof. Certain identified clusters are described herein. Certain MDM2 binding agents are presented below as examples. m*=D-Nle (dnL).

| Cluster | ID | Description (All D-amino acids) | Measured K_{D} (uM) |
|---|---|---|---|
| MDM2.C1 | H101^{†} | Ac-dpahwycdfaaqvenfs-NH₂ (SEQ ID NO: 4) | 5.5* |
| MDM2.C2 | H102^{†} | Ac-dpawnhceyaafvcsev-NH₂ (SEQ ID NO: 5) | 2.7 |
| MDM2.C3 | H103^{†} | Ac-dpawyecm*eaam*m*cqqv-NH₂ (SEQ ID NO: 6) | 0.88 |
| MDM2.C4 | H104 | Ac-dpalvecnlaadm*chfy-NH₂ (SEQ ID NO: 7) | 1.8 |
| MDM2.C5 | H105 | Ac-dpaantciwaaiecsm*y-NH₂ (SEQ ID NO: 8) | 16* |
| MDM2.C6 | H106 | Ac-dpawatcm*daalnclqm*- NH₂ (SEQ ID NO: 9) | 1.8 |
| MDM2.C7 | H107 | Ac-dpasrncqwaahlceyw-NH₂ (SEQ ID NO: 10) | 1.1 |
| MDM2.C8 | H108 | Ac-dpalaqcrwaawrcdfe-NH₂ (SEQ ID NO: 11) | 12* |
| MDM2.C9 | H109 | Ac-dpaswqcvm*aam*dcvld-NH₂ (SEQ ID NO: 12) | 2.4 |

Peptides were cyclized between C7 and C14 with N, N′-(1,4-phenylene)bis(2-bromoacetamide). * Listed K_{D} values are beyond tested concentrations. * Sequences were selected for co-crystallization with MDM2.

SPR experiments were performed on a Biacore 8K (Cytiva) instrument at 25° C. in 1×HBS-P+ buffer (Cytiva) with 1% DMSO. A SA Series S sensor chip was docked and pre-conditioned with three injections of 50 mM NaOH/1 M NaCl to remove unbound streptavidin from the surface. Biotinylated MDM2²⁵⁻¹⁰⁹ were diluted to 2 μg/mL in running buffer and immobilized to channels 1 through 8 at 5 μL/min for 71 sec for a final immobilization level of ˜390 RU. Peptides were diluted to 5 μM in running buffer then serially diluted 2-fold for a total of seven concentrations with one blank (7-point two-fold peptide dilution series with top concentration=5 μM and bottom concentration=78 nM). Compounds were injected over the immobilized and reference surfaces at 30 μL/min for 60 sec and then allowed to dissociate for 180 sec without surface regeneration (N=1). Data was analyzed using Biacore Insight Evaluation software (Cytiva). Sensorgrams were double referenced, with most of them fitted to a 1:1 steady state affinity model, with a few fitted with both steady state affinity model and kinetics model.

MDM2 expression: MDM2 (residues 25-109) with an N-terminal 6×His-yBBr-TEV tag was recombinantly expressed in E. coli BL21 CodonPlus cells (Agilent) from pET-derived expression vectors (Novagen) (“6×His” disclosed as SEQ ID NO: 13). The cells were induced at OD600=0.6 with 1 mM isopropyl β-D-1-thiogalactopyranoside (IPTG) for 4 hours at 37° C., then harvested and resuspended in 25 mM Tris-HCl pH 7.5, 300 mM NaCl, 10% glycerol, 1 mM PMSF. For purification, the pellet was lysed with a tip sonicator, and pelleted at 22,000×g for 30 minutes at 4° C. The pellets were washed with 20 mM Tris-HCl pH 8.0, 150 mM NaCl, 1 M urea, 1.0% triton X-100 three times, and the inclusion body was dissolved in in 20 mM Tris-HCl pH 8.0, 150 mM NaCl, 8 M urea, and 2 mM ß-mercaptoethanol (ß-me). The supernatant was purified with Ni-NTA resin (Qiagen), eluting with 20 mM Tris-HCl pH 8.0, 150 mM NaCl, 8 M urea, 2 mM ß-me, and 250 mM imidazole. Protein elutes were diluted to ˜0.1 mg/mL and dialyzed into buffers containing 10 mM Tris-HCl pH 8.0, 150 mM NaCl, 2 mM ß-me, with 4, 2, 1, or 0 M urea, at 4° C. for 8 hours for each urea gradient. Urea-free proteins were concentrated with Amicon spin filters (Millipore Sigma) to ˜1 mg/mL and biotinylated via the yBBr reaction according to reported procedures (e.g., reported in Yin, J. et al. Genetically encoded short peptide tag for versatile protein labeling by Sfp phosphopantetheinyl transferase. Proceedings of the National Academy of Sciences 102, 15815-15820 (2005)). Biotinylated proteins were pooled, concentrated, and loaded onto a Superdex™ 10/300 75 pg (Cytiva) SEC column and eluted in 20 mM HEPES pH 7.0, 200 mM NaCl, 5% glycerol, 0.5 mM tris(2-carboxyethyl) phosphine (TCEP). Fractions containing pure protein were collected, pooled, concentrated to ˜1 mg/mL and stored at −80° C. In some embodiments, such MDM2 preparation were utilized in biochemical assays.

In some embodiments, MDM2 polypeptide was prepared as follows: MDM2 (residues 17-111, C17S) with an N-terminal 6×His-TEV tag was recombinantly expressed in E. coli BL21 (DE3) cells (Agilent) from pET-derived expression vectors (Novagen) (“6×His” disclosed as SEQ ID NO: 13). The cells were induced at OD600=0.6 with 0.15 mM IPTG for 16 hours at 37° C., then harvested and resuspended in 50 mM Tris, pH 8.0, 200 mM NaCl, 10% glycerol, 1 mM TCEP, and 20 mM imidazole. For purification, the pellet was lysed with a tip sonicator with power level set at 200 W, 3 seconds on and 3 seconds off for 20 min, pelleted at 22,000×g for 30 minutes at 4° C. The supernatant was purified with Ni-NTA resin (Qiagen), eluting with 50 mM Tris, pH 8.0, 200 mM NaCl, 10% glycerol, 1 mM TCEP, and 250 mM imidazole. Eluted proteins were pooled, concentrated, and TEV-cleaved by adding TEV protease at a ratio of 1:10 protease to protein and incubated overnight at 4° C. TEV-cleaved proteins were loaded onto a Superdex™ 10/300 75 pg (Cytiva) SEC column and eluted in 50 mM Tris, pH 8.0, 200 mM NaCl, 10% glycerol, and 1 mM TCEP. Fractions containing pure protein were collected, pooled, concentrated to 8 mg/mL and stored at −80° C. In some embodiments, such MDM2 preparation were utilized for crystallography.

Interactions between provided agents and MDM2 were also demonstrated by crystallography. Certain results were provided below as examples. To obtain structures of protein-peptide complexes, briefly, 10 mM peptides stock in 90% DMSO were added to protein stocks to a final 1:1.25 protein: peptide molar ratio and screened against commercially available crystallization screens. Crystals were obtained by sitting hanging drop vapor diffusion methods at room temperature, with their crystallization conditions detailed in relevant tables. Crystals were cryo protected with glycerol or ethylene glycol followed by flash-freezing in liquid nitrogen. Diffraction datasets were collected at 100K at a variety of sources as described in the tables. Data was processed in XDS, autoPROC, and AIMLESS. Molecular replacement solutions were obtained using PHASER with previously deposited high resolution PDB structures as search models. Complete models were built through iterative cycles of manual model building in COOT and structure refinement using REFMAC and PHENIX. All the structure model figures in the paper were prepared using PyMOL (The PyMOL Molecular Graphics System, Version 2.4, Schrödinger, LLC.). The atomic coordinates and structure factors have been deposited in the Protein Data Bank, www.pdb.org.

Crystals were obtained by either the sitting hanging drop or hanging drop vapor diffusion methods at room temperature. Crystals were cryo protected followed by flash-freezing in liquid nitrogen. Diffraction datasets were collected at 100 K at a variety of sources. Data was processed in XDS, autoPROC and/or STARANISO. Molecular replacement solutions were obtained using PHASER with previously deposited high resolution PDB structures as search models. Complete models were built through iterative cycles of manual model building in COOT and structure refinement using either REFMAC or PHENIX. The atomic coordinates and structure factors have been deposited in the Protein Data Bank, www.pdb.org. In some embodiments, stapled peptide agents formed left-handed (α-helices to engage CHIP.

MDM2-H101 Data Collection and Refinement Statistics

| Title | MDM2_H101 | |
|---|---|---|
| PDB Entry | 8F0Z | |
| Description | Structure of the MDM2 P53 binding | |
| | domain in complex with H101, an | |
| | all-D Helicon Polypeptide | |
| Wavelength (Å) | 1.03319 | |
| Resolution range (Å) | 40.87-1.61 | (1.64-1.61) |
| Space group | P 21 21 2 | |
| Unit cell: a, b, c (Å), α, β, γ (°) | 40.87 81.36 34.98 90 90 90 | |
| Total reflections | 200195 | (10013) |
| Unique reflections | 15696 | (774) |
| Multiplicity | 12.8 | (12.9) |
| Completeness (%) | 99.6 | (100.0) |
| Mean I/sigma(I) | 19.3 | (2.1) |
| Wilson B-factor (Å²) | 27.45 | |
| R-merge | 0.067 | (1.18) |
| R-meas | 0.072 | (1.28) |
| R-pim | 0.028 | (0.48) |
| CC1/2 | 0.998 | (0.774) |
| Reflections used in refinement | 15649 | (1542) |
| Reflections used for R-free | 756 | (74) |
| R-work | 0.2209 | (0.3047) |
| R-free | 0.2423 | (0.3188) |

| Number of non-hydrogen atoms | 1006 |
|---|---|
| macromolecules | 923 |
| ligands | 16 |
| solvent | 67 |
| Protein residues | 104 |
| RMS(bonds) (Å) | 0.009 |
| RMS(angles) (°) | 1.33 |
| Ramachandran favored (%) | 100 |
| Ramachandran allowed (%) | 0 |
| Ramachandran outliers (%) | 0 |
| Rotamer outliers (%) | 4.40 |
| Clashscore | 7.97 |
| Average B-factor (Å²) | 35.27 |
| macromolecules | 34.61 |
| ligands | 31.76 |
| solvent | 45.08 |
| Crystallization conditions | 3.5M Sodium Formate pH 7.0 |
| Beamline | APS, 23-ID-B |

MDM2-H102 Data Collection and Refinement Statistics

| Title | MDM2_H102 | |
|---|---|---|
| PDB Entry | 8F10 | |
| Description | Structure of the MDM2 P53 binding | |
| | domain in complex with H102, an | |
| | all-D Helicon Polypeptide | |
| Wavelength (Å) | 0.97933 | |
| Resolution range (Å) | 42.17-1.28 | (1.30-1.28) |
| Space group | P 1 21 1 | |
| Unit cell: a, b, c (Å), α, β, γ (°) | 28.15 39.92 42.36 90 95.44 90 | |
| Total reflections | 99690 | (439) |
| Unique reflections | 20614 | (333) |
| Multiplicity | 4.8 | (1.3) |
| Completeness (%) | 85.3 | (29.1) |
| Mean I/sigma(I) | 18.2 | (2.1) |
| Wilson B-factor (Å²) | 10.14 | |
| R-merge | 0.039 | (0.30) |
| R-meas | 0.049 | (0.42) |
| R-pim | 0.029 | (0.30) |
| CC1/2 | 0.999 | (0.909) |
| Reflections used in refinement | 20091 | (736) |
| Reflections used for R-free | 968 | (38) |
| R-work | 0.1587 | (0.2479) |
| R-free | 0.1708 | (0.2064) |

| Number of non-hydrogen atoms | 1062 |
|---|---|
| macromolecules | 908 |
| ligands | 49 |
| solvent | 105 |
| Protein residues | 102 |
| RMS(bonds) (Å) | 0.013 |
| RMS(angles) (°) | 1.33 |
| Ramachandran favored (%) | 97.65 |
| Ramachandran allowed (%) | 2.35 |
| Ramachandran outliers (%) | 0 |
| Rotamer outliers (%) | 0 |
| Clashscore | 10.35 |
| Average B-factor (Å²) | 17.25 |
| macromolecules | 15.16 |
| ligands | 26.08 |
| solvent | 31.17 |
| Crystallization conditions | 0.01M tri-Sodium citrate, |
| | 33% w/v PEG 6000 |
| Beamline | NSLS II, 17-ID-2 FMX |

MDM2-H103 Data Collection and Refinement Statistics

| Title | MDM2_H103 | |
|---|---|---|
| PDB Entry | 8F12 | |
| Description | Structure of the MDM2 P53 binding | |
| | domain in complex with H103, an | |
| | all-D Helicon Polypeptide | |
| Wavelength (Å) | 1.03317 | |
| Resolution range (Å) | 49.11-1.86 | (1.90-1.86) |
| Space group | P 43 21 2 | |
| Unit cell: a, b, c (Å), α, β, γ (°) | 60.31 60.31 84.59 90 90 90 | |
| Total reflections | 354507 | (19344) |
| Unique reflections | 13715 | (817) |
| Multiplicity | 25.8 | (23.7) |
| Completeness (%) | 99.8 | (98.2) |
| Mean I/sigma(I) | 18.0 | (2.2) |
| Wilson B-factor (Å²) | 25.46 | |
| R-merge | 0.113 | (1.58) |
| R-meas | 0.117 | (1.64) |
| R-pim | 0.031 | (0.46) |
| CC1/2 | 0.999 | (0.851) |
| Reflections used in refinement | 13668 | (1318) |
| Reflections used for R-free | 661 | (54) |
| R-work | 0.2172 | (0.2821) |
| R-free | 0.2422 | (0.3280) |

| Number of non-hydrogen atoms | 1073 |
|---|---|
| macromolecules | 971 |
| ligands | 25 |
| solvent | 77 |
| Protein residues | 111 |
| RMS(bonds) (Å) | 0.014 |
| RMS(angles) (°) | 1.38 |
| Ramachandran favored (%) | 96.77 |
| Ramachandran allowed (%) | 3.23 |
| Ramachandran outliers (%) | 0 |
| Rotamer outliers (%) | 1.03 |
| Clashscore | 4.01 |
| Average B-factor (Å²) | 37.11 |
| macromolecules | 35.83 |
| ligands | 54.29 |
| solvent | 47.68 |
| Crystallization conditions | 3.2M Ammonium sulfate, |
| | 0.1M Citrate pH 5.0 |
| Beamline | APS, 23-ID-B |

MDM2-H103-Alt Data Collection and Refinement Statistics

| Title | MDM2_H103-Alt | |
|---|---|---|
| PDB Entry | 8F13 | |
| Description | Structure of the MDM2 P53 binding | |
| | domain in complex with H103, an | |
| | all-D Helicon Polypeptide, | |
| | alternative C-terminus | |
| Wavelength (Å) | 1.03319 | |
| Resolution range (Å) | 42.45-1.40 | (1.42-1.40) |
| Space group | P 43 21 2 | |
| Unit cell: a, b, c (Å), α, β, γ (°) | 60.29 60.29 84.90 90 90 90 | |
| Total reflections | 645852 | (13067) |
| Unique reflections | 31020 | (1334) |
| Multiplicity | 20.8 | (9.8) |
| Completeness (%) | 98.4 | (86.0) |
| Mean I/sigma(I) | 23.0 | (2.3) |
| Wilson B-factor (Å²) | 19.45 | |
| R-merge | 0.083 | (1.27) |
| R-meas | 0.087 | (1.42) |
| R-pim | 0.025 | (0.61) |
| CC1/2 | 0.996 | (0.771) |
| Reflections used in refinement | 30774 | (2630) |
| Reflections used for R-free | 1526 | (121) |
| R-work | 0.1744 | (0.3414) |
| R-free | 0.2078 | (0.3765) |

| Number of non-hydrogen atoms | 1175 |
|---|---|
| macromolecules | 974 |
| ligands | 51 |
| solvent | 150 |
| Protein residues | 112 |
| RMS(bonds) (Å) | 0.009 |
| RMS(angles) (°) | 1.44 |
| Ramachandran favored (%) | 97.85 |
| Ramachandran allowed (%) | 2.15 |
| Ramachandran outliers (%) | 0 |
| Rotamer outliers (%) | 6.25 |
| Clashscore | 7.27 |
| Average B-factor (Å²) | 29.64 |
| macromolecules | 26.74 |
| ligands | 37.87 |
| solvent | 45.69 |
| Crystallization conditions | 0.2M Potassium sodium tartrate, |
| | 0.1M tri-Sodium citrate pH 5.6, |
| | 2.0M Ammonium sulfate |
| Beamline | APS, 23-ID-B |

### Example 2. The Present Disclosure Provides Agents that Bind CHIP

Among other things, the present disclosure provides CHIP-binding agents and compositions thereof. Certain identified clusters are described herein. Certain CHIP binding agents are presented below as examples.

| Cluster | ID | Description (All D-amino acids) | Measured K_{D} |
|---|---|---|---|
| CHIP.C1 | H201^{†} | Ac-pwedcawfawacyni-NH2 (SEQ ID NO: 14) | 470 nM |
| CHIP.C3 | H202^{†} | Ac-pwweclsqaddcdfr-NH2 (SEQ ID NO: 15) | 1230 M |
| CHIP.C2 | H203^{†} | Ac-phemcywadaycrys-NH2 (SEQ ID NO: 16) | 550 nM |
| CHIP.C2 | H204^{†} | Ac-pldlcywaslhcivs-NH2 (SEQ ID NO: 17) | 770 nM |

Peptides were cyclized between C7 and C14 with N, N′-(1,4-phenylene)bis(2-bromoacetamide). * Sequences were selected for co-crystallization with MDM2.

For the competition FP of CHIP, stapled peptides at 10 mM in DMSO were serially diluted 1:3 in DMSO for a total of 11 concentrations using a Mosquito LV (SPT Labtech), then diluted 1000-fold in buffer (1×HBS-P+, Cytiva) in duplicate by the Mosquito LV (SPT Labtech) into a black polystyrene 384-well plate (Corning) (11-point three-fold peptide dilution series with top concentration=10 μM). The assay was performed with 400 nM CHIP23-303 recombinant protein as target, and 20 nM BCHIP-binding peptide as probe (5FAM-bAla-SSGPTIEEVD (SEQ ID NO: 18), derived from HSP70). The plate was incubated and protected from light for 1 hour at room temperature prior to reading. Reads were performed on a CLARIOstar plate reader (BMG Labtech) with excitation at 485 nm, emission at 525 nm, and cutoff at 504 nm. Data were fitted to a 1:1 binding model with Hill slope using an in-house script.

In some embodiments, CHIP polypeptide was prepared as follows: CHIP (STUB1, residues 23-154) with an N-terminal 6×His-yBBr-TEV tag was recombinantly expressed in E. coli BL21 CodonPlus cells (Agilent) from pET-derived expression vectors (Novagen) (“6×His” disclosed as SEQ ID NO: 13). The cells were induced at OD600=0.6 with 1 mM IPTG for 4 hours at 37° C., then harvested and resuspended in 50 mM Tris-HCl pH 8.0, 500 mM NaCl, 10 mM imidazole, 10% glycerol, and 10 mM ß-me. For purification, the pellet was lysed with a tip sonicator and pelleted at 22,000×g for 30 minutes at 4° C. The supernatant was purified with Ni-NTA resin (Qiagen), eluted with 50 mM Tris-HCl pH 8.0, 500 mM NaCl, 10% glycerol, 10 mM ß-me and 250 mM imidazole, and biotinylated via the yBBr reaction according to reported procedures (e.g., reported in Yin, J. et al. Genetically encoded short peptide tag for versatile protein labeling by Sfp phosphopantetheinyl transferase. Proceedings of the National Academy of Sciences 102, 15815-15820 (2005)). Biotinylated proteins were pooled, concentrated, and loaded onto a Superdex™ 10/300 75 pg (Cytiva) SEC column, and eluted in 20 mM HEPES pH 7.0, 150 mM NaCl, 10% glycerol, 2 mM DTT. Fractions containing pure protein were collected, pooled, concentrated to ˜1.2 mg/mL and stored at −80° C. In some embodiments, such CHIP preparations were utilized in phage screen and biochemical assays.

In some embodiments, CHP polypeptide was prepared as follows: CHIP (residues 23-303 for competition FP and 23-154 for crystallography) with an N-terminal 6×His-TEV tag was recombinantly expressed in E. coli BL21 CodonPlus cells (Agilent) from a pET21b-derived expression vector (Novagen) (“6×His” disclosed as SEQ ID NO: 13). The cells were induced at OD600=0.6 with 1 mM IPTG for 4 hours at 37° C., then harvested and resuspended in 50 mM Tris-HCl pH 8.0, 500 mM NaCl, 10 mM imidazole, 10% glycerol, and 10 mM ß-me. For purification, the pellet was lysed with a tip sonicator with power level set at 400 W, 3 seconds on and 3 seconds off for 20 min, pelleted at 22,000×g for 30 minutes at 4° C. The supernatant was purified with Ni-NTA resin (Qiagen), eluting with 250 mM imidazole. Eluted proteins were pooled, concentrated, and TEV-cleaved by adding TEV protease at a ratio of 1:10 protease to protein and incubated overnight at 4° C. TEV-cleaved proteins were loaded onto a Superdex™ 10/300 75 pg (Cytiva) SEC column, and eluted in 50 mM HEPES, pH 8.0, 150 mM NaCl, 10% glycerol, 2 mM DTT. Fractions containing pure protein were collected, pooled, concentrated to 30 mg/mL and stored at −80° C. In some embodiments, such CHIP preparations were utilized in competition FP and crystallography.

Interactions between provided agents and CHIP were also demonstrated by crystallography. Certain results were provided below as examples. In some embodiments, stapled peptide agents formed left-handed α-helices to engage CHIP.

CHIP-H201 Data Collection and Refinement Statistics

| Title | CHIP06_H201 | |
|---|---|---|
| PDB Entry | 8F14 | |
| Description | Structure of the STUB1 TPR domain | |
| | in complex with H201, an all-D | |
| | Helicon Polypeptide | |
| Wavelength (Å) | 0.89685 | |
| Resolution range (Å) | 92.33-1.69 | (1.72-1.69) |
| Space group | C 2 2 21 | |
| Unit cell: a, b, c (Å), α, β, γ (°) | 46.16 90.12 92.33 90 90 90 | |
| Total reflections | 296534 | (14797) |
| Unique reflections | 21997 | (1103) |
| Multiplicity | 13.5 | (13.4) |
| Completeness (%) | 100.0 | (100.0) |
| Mean I/sigma(I) | 20.2 | (2.2) |
| Wilson B-factor (Å²) | 23.75 | |
| R-merge | 0.069 | (1.22) |
| R-meas | 0.075 | (1.32) |
| R-pim | 0.028 | (0.50) |
| CC1/2 | 0.999 | (0.850) |
| Reflections used in refinement | 21945 | (2153) |
| Reflections used for R-free | 1106 | (116) |
| R-work | 0.1967 | (0.2927) |
| R-free | 0.2351 | (0.3368) |

| Number of non-hydrogen atoms | 1422 |
|---|---|
| macromolecules | 1256 |
| ligands | 32 |
| solvent | 134 |
| Protein residues | 149 |
| RMS(bonds) (Å) | 0.011 |
| RMS(angles) (°) | 1.20 |
| Ramachandran favored (%) | 98.46 |
| Ramachandran allowed (%) | 1.54 |
| Ramachandran outliers (%) | 0 |
| Rotamer outliers (%) | 2.63 |
| Clashscore | 2.78 |
| Average B-factor (Å²) | 34.98 |
| macromolecules | 34.11 |
| ligands | 39.38 |
| solvent | 42.10 |
| Crystallization conditions | 0.2M Lithium Sulfate, |
| | 0.1M Bis-Tris pH 6.5, |
| | 25% w/v PEG 3350 |
| Beamline | Diamond I03 |

CHIP-H202 Data Collection and Refinement Statistics

| Title | CHIP06_H202 | |
|---|---|---|
| PDB Entry | 8F15 | |
| Description | Structure of the STUB1 TPR domain | |
| | in complex with H202, an all-D | |
| | Helicon Polypeptide | |
| Wavelength (Å) | 1.00000 | |
| Resolution range (Å) | 49.02-1.73 | (1.76-1.73) |
| Space group | P 43 | |
| Unit cell: a, b, c (Å), α, β, γ (°) | 50.56 50.56 199.85 90 90 90 | |
| Total reflections | 721070 | (40585) |
| Unique reflections | 51998 | (2848) |
| Multiplicity | 13.9 | (14.3) |
| Completeness (%) | 100.0 | (100.0) |
| Mean I/sigma(I) | 16.6 | (2.2) |
| Wilson B-factor (Å²) | 17.46 | |
| R-merge | 0.102 | (1.24) |
| R-meas | 0.110 | (1.33) |
| R-pim | 0.042 | (0.50) |
| CC1/2 | 0.999 | (0.785) |
| Reflections used in refinement | 51994 | (5201) |
| Reflections used for R-free | 2477 | (287) |
| R-work | 0.1800 | (0.2395) |
| R-free | 0.2096 | (0.2729) |

| Number of non-hydrogen atoms | 4113 |
|---|---|
| macromolecules | 3702 |
| ligands | 79 |
| solvent | 332 |
| Protein residues | 448 |
| RMS(bonds) (Å) | 0.014 |
| RMS(angles) (°) | 1.43 |
| Ramachandran favored (%) | 98.73 |
| Ramachandran allowed (%) | 1.27 |
| Ramachandran outliers (%) | 0 |
| Rotamer outliers (%) | 3.78 |
| Clashscore | 3.91 |
| Average B-factor (Å²) | 27.58 |
| macromolecules | 26.55 |
| ligands | 30.81 |
| solvent | 38.32 |
| Crystallization conditions | 0.1M TRIS pH 8, 30% v/v PEG 400 |
| Beamline | Spring8 BL45XU |

CHIP-H203 Data Collection and Refinement Statistics

| Title | CHIP06_H203 | |
|---|---|---|
| PDB Entry | 8F16 | |
| Description | Structure of the STUB1 TPR domain | |
| | in complex with H203, an all-D | |
| | Helicon Polypeptide | |
| Wavelength (Å) | 1.18057 | |
| Resolution range (Å) | 45.41-1.56 | (1.59-1.56) |
| Space group | P 21 21 21 | |
| Unit cell: a, b, c (Å), α, β, γ (°) | 50.56 66.88 103.28 90 90 90 | |
| Total reflections | 315958 | (16165) |
| Unique reflections | 50341 | (2511) |
| Multiplicity | 6.3 | (6.4) |
| Completeness (%) | 99.5 | (100.0) |
| Mean I/sigma(I) | 22.4 | (2.2) |
| Wilson B-factor (Å²) | 22.78 | |
| R-merge | 0.030 | (0.68) |
| R-meas | 0.036 | (0.80) |
| R-pim | 0.019 | (0.43) |
| CC1/2 | 1.000 | (0.893) |
| Reflections used in refinement | 50091 | (5005) |
| Reflections used for R-free | 2436 | (257) |
| R-work | 0.1979 | (0.2629) |
| R-free | 0.2302 | (0.3036) |

| Number of non-hydrogen atoms | 2942 |
|---|---|
| macromolecules | 2556 |
| ligands | 60 |
| solvent | 326 |
| Protein residues | 301 |
| RMS(bonds) (Å) | 0.011 |
| RMS(angles) (°) | 1.25 |
| Ramachandran favored (%) | 99.23 |
| Ramachandran allowed (%) | 0.77 |
| Ramachandran outliers (%) | 0 |
| Rotamer outliers (%) | 3.39 |
| Clashscore | 5.83 |
| Average B-factor (Å²) | 28.91 |
| macromolecules | 27.74 |
| ligands | 34.66 |
| solvent | 37.03 |
| Crystallization conditions | 0.1M BICINE pH 8.5, |
| | 15% w/v PEG 1,500 |
| Beamline | CLS, 08B1 |

CHIP-H204 Data Collection and Refinement Statistics

| Title | CHIP06_H204 | |
|---|---|---|
| PDB Entry | 8F17 | |
| Description | Structure of the STUB1 TPR domain | |
| | in complex with H204, an all-D | |
| | Helicon Polypeptide | |
| Wavelength (Å) | 1.18057 | |
| Resolution range (Å) | 46.01-2.21 | (2.28-2.21) |
| Space group | P 1 21 1 | |
| Unit cell: a, b, c (Å), α, β, γ (°) | 38.03 72.71 59.79 90 96.31 90 | |
| Total reflections | 53489 | (4781) |
| Unique reflections | 16124 | (1388) |
| Multiplicity | 3.3 | (3.4) |
| Completeness (%) | 99.0 | (98.6) |
| Mean I/sigma(I) | 16.9 | (2.3) |
| Wilson B-factor (Å²) | 38.8 | |
| R-merge | 0.032 | (0.40) |
| R-meas | 0.045 | (0.56) |
| R-pim | 0.032 | (0.40) |
| CC1/2 | 0.999 | (0.883) |
| Reflections used in refinement | 15189 | (1493) |
| Reflections used for R-free | 764 | (84) |
| R-work | 0.2166 | (0.2997) |
| R-free | 0.2523 | (0.3046) |

| Number of non-hydrogen atoms | 2426 |
|---|---|
| macromolecules | 2338 |
| ligands | 36 |
| solvent | 52 |
| Protein residues | 292 |
| RMS(bonds) (Å) | 0.003 |
| RMS(angles) (°) | 0.51 |
| Ramachandran favored (%) | 97.64 |
| Ramachandran allowed (%) | 2.36 |
| Ramachandran outliers (%) | 0 |
| Rotamer outliers (%) | 1.40 |
| Clashscore | 4.70 |
| Average B-factor (Å²) | 50.27 |
| macromolecules | 50.33 |
| ligands | 50.91 |
| solvent | 47.19 |
| Crystallization conditions | 0.15M Sodium chloride, 0.1M |
| | TRIS pH 8, 8% w/v PEG 6000 |
| Beamline | CLS, 08B1 |

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described in the present disclosure. It is, therefore, to be understood that the foregoing embodiments are presented by way of example only and that, provided technologies, including those to be claimed, may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1-508. (canceled)

509. An agent, comprising:
X⁴¹X²X³X⁴X⁵X⁶X⁷X⁸X⁹X¹⁰X¹¹X¹²X¹³X¹⁴,
wherein:
each of X⁴¹, X², X³, X⁴, X⁵, X⁶, X⁷, X⁸, X⁹, X¹⁰, X¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X² comprises a side chain comprising an aromatic group or a hydrophobic side chain; and
X⁶ or X⁷ comprises a hydrophobic side chain or a side chain comprising an aromatic group; or
an agent, comprising:
X⁴²X²X³X⁴X⁵X⁶X⁷X⁸X⁹X¹⁰X¹¹X¹²X¹³X¹⁴,
wherein:
each of X⁴², X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X⁴² comprises a side chain comprising an aromatic group or a hydrophobic side chain; and
X⁶ or X⁷ comprises a hydrophobic side chain or a side chain comprising an aromatic group; or
an agent, comprising:
X⁴³X²X³X⁴X⁵X⁶X⁷X⁸X⁹X¹⁰X¹¹X¹²X¹³X¹⁴,
wherein:
each of X⁴³, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X⁴³ comprises a side chain comprising an aromatic group or a hydrophobic side chain; and
X⁵ comprises a hydrophobic side chain.

510. The agent of claim 509, comprising:
X⁴¹X²X³X⁴X⁵X⁶X⁷X⁸X⁹X¹⁰X¹¹X¹²X¹³X¹⁴,
wherein:
each of X⁴¹, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X² comprises a side chain comprising an aromatic group or a hydrophobic side chain; and
X⁶ or X⁷ comprises a hydrophobic side chain or a side chain comprising an aromatic group.

511. The agent of claim 509, wherein X⁴ or X¹¹ is an amino acid residue suitable for stapling.

512. The agent of claim 509, comprising:
X⁴²X²X³X⁴X⁵X⁶X⁷X⁸X⁹X¹⁰X¹¹X¹²X¹³X¹⁴,
wherein:
each of X⁴², X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X⁴² comprises a side chain comprising an aromatic group or a hydrophobic side chain; and
X⁶ or X⁷ comprises a hydrophobic side chain or a side chain comprising an aromatic group.

513. The agent of claim 509, comprising:
X⁴³X²X³X⁴X⁵X⁶X⁷X⁸X⁹X¹⁰X¹¹X¹²X¹³X¹⁴,
wherein:
each of X⁴³, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X⁴³ comprises a side chain comprising an aromatic group or a hydrophobic side chain; and
X⁵ comprises a hydrophobic side chain.

514. A pharmaceutical composition, comprising or delivering an agent of claim 509 and a pharmaceutically acceptable carrier.

515. A method for modulating a MDM2 function in a system, comprising administering or delivering to the system an effective amount of an agent of claim 509.

516. The method of claim 515, wherein the system expresses a MDM2 polypeptide.

517. A method for treating in a subject or preventing in a subject susceptible thereto a condition, disorder or disease, comprising administering or delivering to a subject an effective amount of an agent of claim 509.

518. The method of claim 516, wherein the condition, disorder or disease is cancer.

519. An agent, comprising:
X³¹X²X³X⁴X⁵X⁶X⁷X⁸X⁹X¹⁰X¹¹X¹²X¹³X¹⁴,
wherein:
each of X³¹, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹, X¹², X¹³, and X¹⁴ is independently a D-amino acid residue;
X³ comprises a side chain comprising an acidic group;
X⁶ comprises a hydrophobic side chain or a side chain comprising an aromatic group;
X⁷ comprises a hydrophobic side chain or a side chain comprising an aromatic group; and
X⁸ comprises a hydrophobic side chain; or
an agent, comprising:
X³²X²X³X⁴X⁵X⁶X⁷X⁸X⁹X¹⁰X¹¹X¹²X¹³X¹⁴,
wherein:
each of X³², X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X² comprises a side chain comprising a polar group;
X³ comprises a hydrophobic side chain; and
X⁶ comprises a hydrophobic side chain or a side chain comprising an aromatic group an agent, comprising:
X³³X²X³X⁴X⁵X⁶X⁷X⁸X⁹X¹⁰X¹¹X¹²X¹³X¹⁴,
wherein:
each of X³³, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X³³ comprises a hydrophobic side chain or a side chain comprising an aromatic group; and
X⁵ comprises a hydrophobic side chain or a side chain comprising an aromatic group, or a side chain comprising a polar group.

520. The agent of claim 519, comprising:
X³¹X²X³X⁴X⁵X⁶X⁷X⁸X⁹X¹⁰X¹¹X¹²X¹³X¹⁴,
wherein:
each of X³¹, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X³ comprises a side chain comprising an acidic group;
X⁶ comprises a hydrophobic side chain or a side chain comprising an aromatic group;
X⁷ comprises a hydrophobic side chain or a side chain comprising an aromatic group; and
X⁸ comprises a hydrophobic side chain.

521. The agent of claim 519, wherein X⁴ or X¹¹ is an amino acid residue suitable for stapling.

522. The agent of claim 519, comprising:
X³²X²X³X⁴X⁵X⁶X⁷X⁸X⁹X¹⁰X¹¹X¹²X¹³X¹⁴,
wherein:
each of X³², X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹¹, X¹², X¹³, and X¹⁴ is independently a D-amino acid residue;
X² comprises a side chain comprising a polar group;
X³ comprises a hydrophobic side chain; and
X⁶ comprises a hydrophobic side chain or a side chain comprising an aromatic group.

523. The agent of claim 519, comprising:
X³³X²X³X⁴X⁵X⁶X⁷X⁸X⁹X¹⁰X¹¹X¹²X¹³X¹⁴,
wherein:
each of X³³, X², X³, X⁴, X⁵, X⁶, X⁷, X⁸, X⁹, X¹⁰, X¹¹, X¹², X¹³, and X¹⁴ is independently an amino acid residue;
at least one of X⁴¹, X², X³, X⁴, X⁵, X⁶, X⁷, X⁸, X⁹, X¹⁰, X¹, X¹², X¹³, and X¹⁴ is independently a D-amino acid residue;
X³³ comprises a hydrophobic side chain or a side chain comprising an aromatic group; and
X⁵ comprises a hydrophobic side chain or a side chain comprising an aromatic group, or a side chain comprising a polar group.

524. A pharmaceutical composition, comprising or delivering an agent of claim 519 and a pharmaceutically acceptable carrier.

525. A method for modulating a CHIP function in a system, comprising administering or delivering to the system an agent of claim 519.

526. The method of claim 525, wherein the system expresses a CHIP polypeptide.

527. A method for treating in a subject or preventing in a subject susceptible thereto a condition, disorder or disease, comprising administering or delivering to a subject an effective amount of an agent of claim 519.

528. The method of claim 527, wherein the condition, disorder or disease is cancer.
